(19) United States
(12) Patent Application Publication
(10) Pub. No.: US 2019/0381127 A1
(43) Pub. Date: Dec. 19, 2019

(54) COMBINATION THERAPY WITH A6, CHEMOTHERAPEUTIC AGENTS, RADIATION THERAPY, OR A COMBINATION THEREOF FOR THE TREATMENT OF CANCER

(71) Applicant: Splash Pharmaceuticals, Inc., San Diego, CA (US)

(72) Inventors: Malcolm Finlayson, San Diego, CA (US); David Nelson, San Diego, CA (US)

(21) Appl. No.: 16/072,134

(22) PCT Filed: Jan. 31, 2017
(86) PCT No.: PCT/US17/15754

§ 371 Date: Jul. 23, 2018

Related U.S. Application Data
(60) Provisional application No. 62/290,306, filed on Feb. 2, 2016.
(60) Provisional application No. 62/314,867, filed on Mar. 29, 2016.
(60) Provisional application No. 62/368,964, filed on Jul. 29, 2016.

(51) Int. Cl. A61K 38/08; A61K 45/06

(52) U.S. Cl. CPC A61K 38/08; A61K 31/337; A61K 45/06

(57) ABSTRACT

Provided herein are therapeutic combinations of A6 peptide (SEQ ID NO: 1) and more anti-cancer agents, radiation therapy, or a combination thereof and the use of such combinations in the treatment of cancer.

### CROSS-REFERENCE TO RELATED APPLICATIONS

This application claims priority to U.S. Provisional Patent Application No. 62/290,306, filed Feb. 2, 2016, U.S. Provisional Patent Application No. 62/314,867, filed Mar. 29, 2016, and to U.S. Provisional Patent Application No. 62/368,964, filed Jul. 29, 2016, each of which are incorporated herein by reference in their entireties and for all purposes.

### FIELD

The present invention relates to combinations of A6 peptide (SEQ ID NO:1) and anti-cancer agents and the use of such combinations in the treatment of cancer. In some aspects, the present invention relates to combinations of A6 peptide (SEQ ID NO:1) and radiation therapy and the use of such combinations in the treatment of cancer.

### REFERENCE TO A SEQUENCE LISTING

This application includes a Sequence Listing created Jan. 30, 2017, and being named “12963-021-228_SequenceListing.txt”, which is 2,086 bytes in size. The material contained in the Sequence Listing is incorporated by reference in its entirety for all purposes.

### BACKGROUND

Mortality due to cancer is generally the result of metastasis of the primary tumor. Recurrence at distant sites following first-line therapy continues to be a major challenge. As a result, drugs that inhibit the metastatic process are of great interest. Metastasis and recurrence have been linked to a subpopulation of highly invasive tumorigenic cells that are characterized by the expression of CD44. There is a need for anti-cancer agents that can inhibit metastasis in cancer patients. Provided herein are solutions to these problems and other problems in the art.

### BRIEF SUMMARY

Provided, inter alia, herein are compositions comprising a CD44-modulating polypeptide described herein and an anti-cancer agent as described herein. The compositions can include a polypeptide of SEQ ID NO:1 or SEQ ID NO:2 and an anti-cancer agent described herein. The compositions are useful in methods of treating cancers as set forth herein.

Further provided herein are compositions comprising a CD44-modulating polypeptide described herein for use in combination with a radiation therapy as described herein. The compositions can include a polypeptide of SEQ ID NO:1 or SEQ ID NO:2. The compositions are useful in methods of treating cancers as set forth herein, particularly in combination with radiation therapy.

In a first aspect is a method of treating cancer in a patient in need thereof by administering an effective amount of a CD44-modulating polypeptide described herein in combination with an effective amount of an anti-cancer agent. In one embodiment, the method includes treating cancer by administering a polypeptide having SEQ ID NO:1 or SEQ ID NO:2 in combination with an effective amount of an anti-cancer agent.

In another aspect is a method of treating cancer in a patient in need thereof by administering an effective amount of a CD44-modulating polypeptide described herein in combination with an effective amount of a radiation therapy. In one embodiment, the method includes treating cancer by administering a polypeptide comprising SEQ ID NO:1 or SEQ ID NO:2 in combination with an effective amount of a radiation therapy.

In another aspect is a pharmaceutical composition that includes a CD44-modulating polypeptide described herein, an anti-cancer agent, and a pharmaceutically acceptable excipient. In still another aspect is a pharmaceutical composition that includes a polypeptide of SEQ ID NO:1, an anti-cancer agent, and a pharmaceutically acceptable excipient. In still another aspect is a pharmaceutical composition that includes a polypeptide of SEQ ID NO:2, an anti-cancer agent, and a pharmaceutically acceptable excipient.

In still another aspect is a pharmaceutical composition that includes a CD44-modulating polypeptide described herein and a pharmaceutically acceptable excipient for use in combination with a radiation therapy. In yet another aspect is a pharmaceutical composition that includes a polypeptide of SEQ ID NO:1 or SEQ ID NO:2 and a pharmaceutically acceptable excipient for use in combination with a radiation therapy.

In any of the various aspects, the CD44-modulating polypeptides described herein and the radiation therapy may be further combined with an anti-cancer agent as provided herein.

### BRIEF DESCRIPTION OF THE DRAWINGS

FIG. 1 illustrates that the connecting peptide domain is located between the N-terminal growth factor domain and the C-terminal catalytic domain of uPA

FIG. 2 illustrates that the polypeptide of SEQ ID NO:1 shares sequence homology with a portion of the Link-Domain of CD44 (CD44 amino acid residues 120-NASAPPEE-127).

FIGS. 3A-3D illustrate testing of whether A6 differentially modulates the sensitivity of cisplatin-sensitive versus cisplatin resistant human ovarian cancer 2008 cells.

FIG. 4 illustrates testing of whether A6 differentially modulates the sensitivity of cisplatin-sensitive versus cisplatin resistant human ovarian cancer 2008 cells measuring using a microplate reader at a wavelength of 450 nM.

FIGS. 5A-5D illustrate testing of whether A6 differentially modulates the sensitivity of cisplatin-sensitive versus cisplatin resistant human ovarian cancer A2780 cells.

FIG. 6 illustrates testing of whether A6 differentially modulates the sensitivity of cisplatin-sensitive versus cisplatin resistant human ovarian cancer A2780 cells measuring using a microplate reader at a wavelength of 450 nM.

FIGS. 7A-7D illustrate testing of whether A6 differentially modulates the sensitivity of cisplatin-sensitive versus cisplatin resistant human ovarian cancer IGROV-1 cells.

FIG. 8 illustrates testing of whether A6 differentially modulates the sensitivity of cisplatin-sensitive versus cisplatin resistant human ovarian cancer IGROV-1 cells measuring using a microplate reader at a wavelength of 450 nM

FIG. 9 illustrates testing of A6 plus Cisplatin (DDP) or Paclitaxel (PTX) in the B16F10-DsRed Cell Lung Metastasis Model. Tumor burden in the lungs was not significantly reduced by A6 in the presence or absence of paclitaxel

FIG. 10 illustrates testing of A6 plus Cisplatin (DDP) or Paclitaxel (PTX) in the B16F10-DsRed Cell Lung Metastasis Model. Tumor burden in the lungs was not significantly reduced by A6 in the presence or absence of cisplatin.

FIG. 11 illustrates testing of A6 plus Cisplatin (DDP) or Paclitaxel (PTX) in HEY (DDP sensitive) and HEY/C2 (DDP resistant) cells.

FIG. 12. Illustrates testing of A6 plus Cisplatin DDP Paclitaxel (PTX) in HEY (DDP sensitive) and HEY/C2 (DDP resistant) cells.

### DETAILED DESCRIPTION

All patents, applications, published applications and other publications are incorporated by reference in their entirety and for all purposes. Unless defined otherwise, all technical and scientific terms used herein have the same meaning as commonly understood by those of ordinary skill in the art to which the invention belongs. The chemical structures and formulae set forth herein are constructed according to the standard rules of chemical valency known in the chemical arts. Should a discrepancy exist between a depicted structure and a name given for that structure, the depicted structure is to be accorded more weight. Where the stereochemistry of a structure or a portion of a structure is not indicated in a depicted structure or a portion of the depicted structure, the depicted structure is to be interpreted as encompassing all of its possible stereoisomers.

Any methods, devices and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

The terms “polypeptide” and “protein” are used interchangeably herein and refer to any molecule that includes at least 2 or more amino acids.

As used herein, “administering” and the like refer to the act physically delivering a composition or other therapy (e.g. a radiation therapy) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. Radiation therapy can be administered using techniques described herein, including for example, external beam radiation or brachytherapy. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

The term “coadministration” refers to administration of two or more agents (e.g., a polypeptide described herein and another active agent such as an anti-cancer agent or other therapy (e.g. a radiation therapy) described herein). The timing of coadministration depends in part on the combination and compositions or other therapies administered and can include administration at the same time, just prior to, or just after the administration of one or more additional therapies, for example cancer therapies such as chemotherapy, hormonal therapy, radiotherapy, or immunotherapy. Coadministration is meant to include simultaneous or sequential administration of a composition or therapy individually or in combination (more than one polypeptide described herein or an anti-cancer agent described herein or radiation therapy as described herein). Coadministration can include administration of two or more agents where the agents are optionally combined with other active substances (e.g., to reduce metabolic degradation). The polypeptides, anti-cancer agents and radiation therapies described herein can be used in combination with one another, with other active agents known to be useful in treating a disease associated with cells expressing a particular kinase as described herein, or with adjunctive agents that cannot be effective alone, but can contribute to the efficacy of the active agent.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances the term patient refers to a human.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

An improvement in the cancer or cancer-related disease can be characterized as a complete or partial response. Complete response refers to an absence of clinically detectable disease with normalization of any previously abnormal radiographic studies, bone marrow, and cerebrospinal fluid (CSF) or abnormal monoclonal protein measurements. Partial response refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

A refractory, resistant, or persistent cancer refers to a circumstance where patients, even after intensive treatment, have residual cancer cells (e.g., leukemia cells, lymphoma cells, circulating tumor cells or cancer stem cells) in their lymphatic system, blood and/or blood forming tissues (e.g., marrow).

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The term “preventing” refers to the treatment with or administration of a polypeptide or agent (e.g. anti-cancer agent described herein) provided herein, with or without other additional active agent (e.g. an anti-cancer agent), prior to the onset of symptoms, particularly to patients at risk of cancer and/or other disorders described herein. The term also refers to coadministration of a polypeptide with other therapies including radiation therapies as described herein. It should be understood that the polypeptides described herein can be coadministered with one or more anti-cancer agents and radiation therapies described herein. The term prevention includes the inhibition or reduction of a symptom of the particular disease, as well as a reduced incidence of a symptom of the particular disease (e.g. by comparison to historical data for a given subject, or population data for similar subjects). Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

A prophylactically effective amount of a polypeptide or agent (e.g. an anti-cancer agent described herein) means an amount of therapeutic agent, alone or in combination with other agents, which provides a prophylactic benefit in the inhibition or reduced incidence of a symptom of a disease or recurrence of a disease. The term also refers to coadministration of a polypeptide described herein with other therapies including radiation therapies as described herein. The term prophylactically effective amount can encompass an amount that improves overall prophylaxis or enhances the prophylactic efficacy of another prophylactic agent.

The term “effective amount” as used herein refers to the amount of a therapy (e.g., a composition or radiation therapy provided herein) which is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount.” of a polypeptide described herein, an anti-cancer agent described herein, or a radiation therapy described herein is an amount sufficient to provide a therapeutic benefit in the treatment or management of a cancer, or to delay or minimize one or more symptoms associated with the presence of the cancer. A therapeutically effective amount of a polypeptide described herein, an anti-cancer agent described herein, or a radiation therapy described herein means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent.

A therapy is any protocol, method and/or agent that can be used in the prevention, management, treatment and/or amelioration of a given disease, disorder or condition. In certain embodiments, the terms “therapies” and “therapy” refer to a drug therapy, biological therapy, supportive therapy, radiation therapy, and/or other therapies useful in the prevention, management, treatment and/or amelioration of a given disease, disorder or condition known to one of skill in the art such as medical personnel.

A regimen is a protocol for dosing and timing the administration of one or more therapies (e.g., combinations described herein, another active agent such as for example an anti-cancer agent described herein, or a radiation therapy described herein) for treating a disease, disorder, or condition described herein. A regimen can include periods of active administration and periods of rest as known in the art. Active administration periods include administration of combinations and compositions described herein and the duration of time of efficacy of such combinations, compositions, and radiation therapies. Rest periods of regimens described herein include a period of time in which no agent (e.g., a polypeptide described herein or an anti-cancer agent described herein) is actively administered, and in certain instances, includes time periods where the efficacy of such agents can be minimal. Rest periods of regimens described herein can include a period of time in which no radiation therapy is actively administered. Combination of active administration and rest in regimens described herein can increase the efficacy and/or duration of administration of the combinations and compositions described herein.

The term “pharmaceutically acceptable” as used herein refers to physiologically acceptable compounds, agents, or ingredients recognized by a regulatory agency of the Federal or state government, or another governmental agency with authorization for such approval, or and an agent listed in the U.S. Pharmacopeia, European Pharmacopeia or other generally recognized Pharmacopeia for use in animals, and more particularly in humans.

A “pharmaceutically acceptable excipient,” refers to a substance that aids the administration of an active agent to a subject by for example modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, sucrose, glucose, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6^{th} Ed. The Pharmaceutical Press, RPS Publishing (2009). The terms binder, tiller, disintegrant, and lubricant are used in accordance with the plain and ordinary meaning within the art.

In certain embodiments, a pharmaceutically acceptable excipient may be incompatible (e.g., cross-reacts) with other excipients or active agents described herein. In some embodiments, magnesium stearate, croscarmellose sodium, lactose, excipients comprising Mg, Ca, K, Li, or Nucleic acid, acesulfame potassium, ammonium alginate, calcium acetate, calcium alginate, calcium carbonate, calcium chloride, calcium lactate, calcium phosphate, calcium silicate, calcium stearate, calcium sulfate, carboxymethylcellulose calcium, carboxymethylcellulose sodium, docusate sodium, glycine, kaolin, magnesium aluminum silicate, magnesium carbonate, magnesium oxide, magnesium silicate, magnesium trisilicate, polacrilin potassium, polymethacrylates, potassium alginate, potassium benzoate, potassium bicarbonate, potassium chloride, potassium citrate, sodium alginate, sodium benzoate, sodium chloride, sodium lauryl sulfate, sodium starch glycolate, sodium stearyl fumarate, sulfobutylether beta-cyclodextrin, sodium stearate, talc, or zinc stearate are incompatible in the dosage forms described herein.

The term “anti-cancer agent” is used in accordance with its plain ordinary meaning and refers to a composition having anti-neoplastic properties or the ability to inhibit the growth or proliferation of cells. In certain embodiments, an anti-cancer agent is a chemotherapeutic. In certain embodiments, an anti-cancer agent is an agent identified herein having utility in methods of treating cancer. In certain embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” or “cancer therapy” refers to a therapy or regimen that includes administration of a combination, chemotherapeutic, or anti-cancer agent described herein.

The term “radiation therapy” is used in accordance with its plain ordinary meaning and refers to the medical use of radiation in the treatment of cancer. Preferably, the medical use of radiation in the treatment of cancer results in the killing of cancer cells in the subject. A variety of radiation therapies can be used in accordance with the present disclosure, examples of which are provided herein.

A “CD44-modulating polypeptide” refers to a polypeptide that binds to CD44 and modulates its activity (e.g., signaling activity). A CD44-modulating polypeptide can be a polypeptide sequence described herein or, in some embodiments, an antibody that specifically binds to CD44 and inhibits its downstream signaling activity. In one embodiment, a CD44-modulating polypeptide can be a polypeptide sequence described herein or, in some embodiments, an antibody that disrupts or inhibits signaling activity of a CD44 dependent co-receptor. In certain instances the CD44 dependent co-receptor is a receptor tyrosine kinase (RTK) such as, for example, Met, Ran, or VEGFR. In still another embodiment a CD44-modulating polypeptide can be a polypeptide sequence described herein or, in some embodiments, an antibody that disrupts CD44 co-receptor function or association of a CD44 co-receptor with CD44 or another signaling protein. In one embodiment, a CD44-modulating polypeptide described herein binds to CD44 and inhibits CD44 signaling activity or association with one or more ABC transporters. The ABC transporter can be a multidrug resistant protein (e.g., MDR1). In certain embodiments, CD44 levels can be elevated upon radiation therapy. Exemplary CD-44 modulating polypeptides include polypeptides having homology to the CD44-v6 region of human CD44. Such peptides can include substitution variants, addition variants, or chemical derivatives thereof including peptidomimetics. In one embodiment, the CD44-modulating polypeptide described herein is a polypeptide having the amino acid sequence of Ac-KPSSPPEE-NH₂ (SEQ ID NO:1), Ac-NASAPPEE-NH₂ (SEQ ID NO:2), QETWFQNGWQGKNP (SEQ ID NO:3), KEKWFENEWQGKNP (SEQ ID NO:4), or KEQWFGNRWHEGYR (SEQ ID NO:5). Another CD44-modulating polypeptide can be QIRQQPRDPPTETLELEVSPDPAS (SEQ ID NO:6). Such exemplary peptides can include substitution variants, addition variants, or chemical derivatives thereof including peptidomimetics. Other exemplary CD44-modulating peptides include those set forth in U.S. Pat. Nos. 5,994,309; 6,696,416; and 6,963,587 and U.S. Patent Application Publication No. US2009192085.

The term “peptidomimetic,” as used herein, means a peptide-like molecule that has the activity of the peptide upon which it is structurally based. Such peptidomimetics include chemically modified peptides, peptide-like molecules containing non-naturally occurring amino acids, and peptoids, and have an activity such as the selective homing activity of the peptide upon which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons (1995), pages 803-861).

The term “prodrug” refers to a compound or polypeptide that is made more active in vivo through metabolism of a precursor drug. CD44-modulating polypeptides described herein can exist as prodrugs, as described in, for example, Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the polypeptides described herein are structurally modified forms of the peptide that readily undergo chemical changes under physiological conditions to provide the active polypeptide. Additionally, prodrugs can be converted to the active polypeptide by chemical or biochemical methods in an ex vivo environment.

A PD-1 inhibitor refers to a moiety (e.g., compound, nucleic acid, polypeptide, antibody) that decreases, inhibits, blocks, abrogates or interferes with the activity or expression of PD-1 (e.g., Programmed Cell Death Protein 1; PD-1 (CD279); GI: 145559515), including variants, isoforms, species homologs of human PD-i (e.g., mouse) and analogs that have at least one common epitope with PD-1. A PD-1 inhibitor includes molecules and macromolecules such as, for example, compounds, nucleic acids, polypeptides, antibodies, peptibodies, diabodies, minibodies, nanobodies, single-chain variable fragments (ScFv), and functional fragments or variants thereof. Thus, a PD-1 inhibitor as used herein refers to any moiety that antagonizes PD-1 activity or expression. PD-1 inhibitor efficacy can be measured, for example, by its inhibitor concentration at 50% (half-maximal inhibitor concentration or IC₅₀). PD-1 inhibitors include exemplary compounds and compositions described herein. A PD-1 antibody refers to a PD-1 inhibitor which is a monoclonal or polyclonal antibody as described herein.

The terms nivolumab, pembrolizumab, pidilizumab, AMP-224, REGN2810, PDR 001, and MEDI0680 are used in accordance with their plain and ordinary meaning as understood in the art.

Provided herein are methods of treating, preventing, managing, or alleviating cancer or symptoms of a cancer in a patient in need thereof. In one aspect is a method of treating a cancer in a patient in need thereof by administering an effective amount of a CD44-modulating polypeptide. In another aspect provided herein is a method of treating cancer in a patient in need thereof by administering an effective amount of a polypeptide comprising the amino acid sequence Ac-KPSSPPEE-NH₂ (SEQ ID NO:1, “A6”) or Ac-NASAPPEE-NH₂ (SEQ ID NO:2) in combination with an effective amount of an anti-cancer agent. In another aspect provided herein is a method of treating cancer in a patient in need thereof by administering an effective amount of a polypeptide consisting of the amino acid sequence of SEQ ID NO:1 or SEQ ID NO:2 in combination with an effective amount of radiation therapy. Amino acids of polypeptides described herein are numbered in reference from their N to C terminal (or its equivalent). For example, SEQ ID NO:1 can be numbered as followed: Ac-K¹P²S³S⁴P⁵P⁶E⁷E⁸-NH₂.

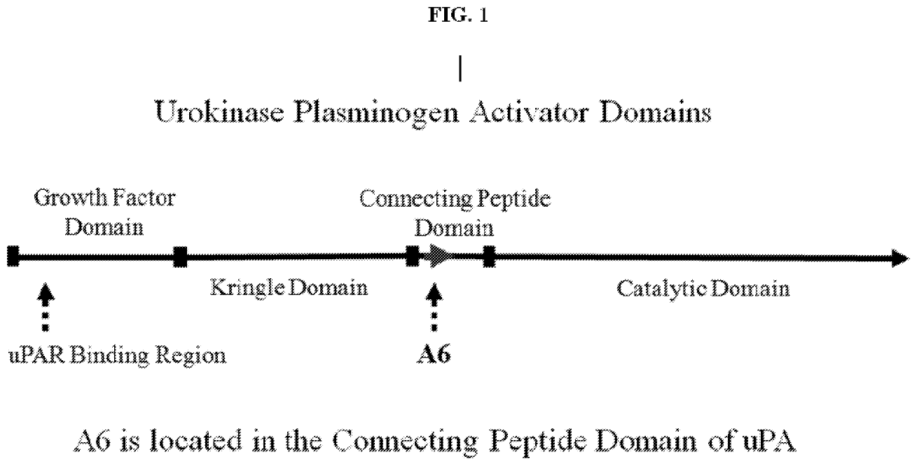

The polypeptide of SEQ ID NO:1 can be a capped 8-amino acid peptide. The sequence of the polypeptide of SEQ ID NO:1 corresponds to amino acid residues 136-143 of the connecting peptide domain of human urokinase plasminogen activator (uPA). The connecting peptide domain is located between the N-terminal growth factor domain and the C-terminal catalytic domain of uPA (FIG. 1). The N-terminal growth factor domain of uPA binds to the uPA receptor (uPAR) to initiate the uPA/uPAR cascade. The binding of uPA to uPAR, can initiate a cascade of events leading to proteolysis, degradation of the extracellular matrix (ECM), cell migration, cell invasion, metastasis, and angiogenesis. Such events can promote cell death, including cell death of cancer cells in solid tumors and hematological cancers. The uPA system has been shown to play a role in the growth and spread of solid tumors. Levels of uPA and uPAR can correlate with clinical outcome in a variety of malignancies. In certain instances, the upregulation of the uPA system can be associated with poor prognosis. The inhibition of the uPA system can block critical processes (e.g., cell migration, invasion, and angiogenesis) useful for a broad range of proliferative diseases.

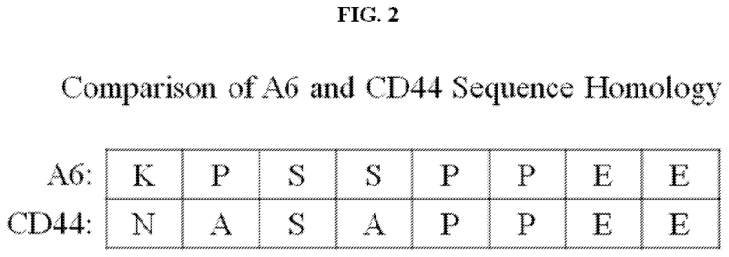

The polypeptide of SEQ ID NO:1 shares sequence homology with a portion of the Link-Domain of CD44 (CD44 amino acid residues 120-NASAPPEE-127) (FIG. 2). The CD44 gene is encoded by 20 exons in the mouse and 19 exons in humans. There are 5 constant exons expressed at the 5′ end, and 10 variant exons (mouse) or 9 variant exons (human) can be alternatively spliced within CD44 at an insertion site after the fifth constitutive exon, followed by the remaining constant exons at the 3′ end. The smallest isoform of CD44 (CD44s) contains no variant exons. The largest isoform of CD44 (CD44v1.-10) contains all of the variant exons. SEQ ID NO:1 can be found nearly all CD44 isoforms, in part, because it is located within the first 5 non-variable exons of the isoform. SEQ ID NO:1 is located at the CD44 splice junction of exons 3 and 4.

The SEQ ID NO:1 can include a substitution of K to A; P to A; S to A; or E to A. In some embodiments, the sequence of SEQ ID NO:1 can be modified such that the CD44-modulating polypeptide includes a mutation of K¹ to A so long as the polypeptide retains activity about equal to or greater than the polypeptide of SEQ ID NO:1. In another embodiment, SEQ ID NO:1 can be modified to include mutation of P², P⁵, P⁶, or a combination thereof to A so long as the polypeptide retains activity about equal to or greater than the polypeptide of SEQ ID NO:1. In certain embodiments, P² can be mutated to A. In certain embodiments, P⁵ can be mutated to A. In certain embodiments, P⁶ can be mutated to A. In another embodiment, S³, S⁴, or S³ and S⁴ can be mutated to A so long as the polypeptide retains activity about equal to or greater than the polypeptide of SEQ ID NO:1. In another embodiment, E⁷, E⁸, or E⁷ and E⁸ can be mutated to A so long as the polypeptide retains activity about equal to or greater than the polypeptide of SEQ ID NO:1. It is to be understood that the above mutations can be combined in any manner to modify the polypeptide of SEQ ID NO:1 so long as the polypeptide retains activity about equal to or greater than the polypeptide of SEQ ID NO:1.

SEQ ID NO:1 can include at least one glycosylation site. The glycosylation site can be an O-linked glycan on S³, S⁴, or S³ and S⁴ of SEQ ID NO:1. In other instances, the glycosylation site can be present in any one Ser or Thr residue of SEQ ID NOs:1-6.

CD44-modulating polypeptides can have anti-migratory activity on cancer cells. In one embodiment, the CD44-modulating polypeptide described herein is a polypeptide comprising or consisting of SEQ ID NO:1 or SEQ ID NO:2 and can have anti-migratory activity on cancer cells. A CD-44 modulating polypeptide can reduce the migratory activity of a cancer cell by at least 2, 3, 4, 5, 10, 15, 20, 25, 30, 35, 40, 45, 50, 55, 60, 65, 70, 75, 80, 85, 90, 95, or 100%. For example, the migratory activity of a cancer cell can be reduced by about: 1% to about 95%, 5% to about 95%, 10 to about 95%, 15% to about 95%, 25% to about 95%, 5% to about 100%, 10% to about 100%, 25% to about 100%; 10% to about 80%, or 10% to about 50%. In another example, the migratory activity can be reduced by at least 5, 10, 20, 25, 30, 40, or 50%. Such reductions can be measured against a control sample or, for example, a baseline sample taken from a subject prior to beginning any treatment described herein.

CD44-modulating polypeptides can have anti-invasive activity on cancer cells. In one embodiment, the CD44-modulating polypeptide described herein is a polypeptide comprising or consisting of SEQ ID NO:1 or SEQ ID NO:2 and can have anti-invasive activity on cancer cells. A CD-44 modulating polypeptide can reduce the invasive activity of a cancer cell by at least 2, 3, 4, 5, 10, 15, 20, 25, 30, 35, 40, 45, 50, 55, 60, 65, 70, 75, 80, 85, 90, 95, or 100%. For example, the invasive activity of a cancer cell can be reduced by about: 1% to about 95%, 5% to about 95%, 10 to about 95%, 15% to about 95%, 25% to about 95%, 5% to about 100%, 10% to about 100%, 25% to about 100%; 10% to about 80%, or 10% to about 50%. In another example, the invasive activity can be reduced by at least 5, 10, 20, 25, 30, 40, or 50%. Such reductions can be measured against a control sample or, for example, a baseline sample taken from a subject prior to beginning any treatment described herein.

CD44-modulating polypeptides can have anti-metastatic activity on cancer cells. In one embodiment, the CD44-modulating polypeptide described herein is a polypeptide comprising or consisting of SEQ ID NO:1 or SEQ ID NO:2 and can have anti-metastatic activity on cancer cells. A CD-44 modulating polypeptide can reduce the metastatic activity of a cancer cell by at least 2, 3, 4, 5, 10, 15, 20, 25, 30, 35, 40, 45, 50, 55, 60, 65, 70, 75, 80, 85, 90, 95, or 100%. For example, the metastatic activity of a cancer cell can be reduced by about: 1% to about 95%, 5% to about 95%, 10 to about 95%, 15% to about 95%, 25% to about 95%, 5% to about 100%, 10% to about 100%, 25% to about 100%; 10% to about 80%, or 10% to about 50%. In another example, the metastatic activity can be reduced by at least 5, 10, 20, 25, 30, 40, or 50%. Such reductions can be measured against a control sample or, for example, a baseline sample taken from a subject prior to beginning any treatment described herein.

Provided herein are methods for treating a resistant or refractory cancer, where the cancer can be resistant to at least one anti-cancer agent or radiation therapy by administering a CD44-modulating polypeptide provided herein in combination with an anti-cancer agent described herein. Also provided herein are methods for treating a resistant or refractory cancer, where the cancer can be resistant to at least one anti-cancer agent or radiation therapy by administering a CD44-modulating polypeptide provided herein in combination with radiation therapy described herein. Further provided herein are methods for treating a resistant or refractory cancer, where the cancer can be resistant to at least one anti-cancer agent or radiation therapy by administering a CD44-modulating polypeptide provided herein in combination with an anti-cancer agent described herein and radiation therapy described herein. It is well known in the art that many cancers are resistant or refractory to many anti-cancer agents or radiation therapy(ies) or over the course of treatment, become resistant or refractory to treatment. The methods described herein can restore activity of anti-cancer agents having reduced or eliminated activity against one or more cancers and permit additional treatment options for cancer patients. In another embodiment, the methods described herein can restore activity of radiation therapies described herein having reduced or eliminated activity against one or more cancers and permit additional treatment options for cancer patients.

The present invention includes embodiments where a CD44-modulating polypeptide described herein establishes, restores or enhances the anti-cancer activity of an anti-cancer agent in treating a cancer that is resistant or refractory to the treatment. In one example, a CD44-modulating polypeptide establishes anti-cancer activity (e.g., creates efficacy of an anti-cancer agent in treating a cancer) of an anti-cancer agent described herein in the treatment of cancer. In another example, a CD44-modulating polypeptide restores the anti-cancer activity of an anti-cancer agent described herein. In another example, a CD44-modulating polypeptide enhances the anti-cancer activity of an anti-cancer agent described herein. In still another example, a combination therapy of a CD44-modulating polypeptide described herein and an anti-cancer agent described herein establishes, restores, or enhances activity of a CD44-rnodulating polypeptide.

The present invention includes embodiments where a CD44-modulating polypeptide described herein establishes, restores or enhances the anti-cancer activity of a radiation therapy in treating a cancer that is resistant or refractory to the treatment. In one example, a CD44-modulating polypeptide establishes anti-cancer activity (e.g., creates efficacy of a radiation therapy) of a radiation therapy described herein in the treatment of cancer. In another example, a CD44-modulating polypeptide restores the anti-cancer activity of a radiation therapy described herein. In another example, a CD44-modulating polypeptide enhances the anti-cancer activity of a radiation therapy described herein. Further provided herein, a CD-44 modulating polypeptide can establish, restore, or enhance the anti-cancer activity of one or more anti-cancer agents and radiation therapy. In still another example, a combination therapy of a CD44-modulating polypeptide described herein and a radiation therapy described herein establishes, restores, or enhances activity of a CD44-modulating polypeptide.

The cancer can optionally be resistant or refractory to a plurality of anti-cancer agents (e.g., two or more anti-cancer agents) and/or a plurality of radiation therapies. In one example the cancer can also be resistant, refractory, or non-responsive to treatment with a CD44-modulating polypeptide described herein. In one embodiment of methods of treating described herein, a patient can be administered a combination of a CD44-modulating polypeptide described herein and an anti-cancer agent where the cancer treated is resistant, refractory, or non-responsive to one of or both the CD44-modulating polypeptide and the anti-cancer agent. In one example, the cancer can be resistant, refractory, or non-responsive to treatment with the anti-cancer agent. Administration of the combination of the CD44-modulating polypeptide and anti-cancer agent(s) surprisingly can restore or enhance the activity of the anti-cancer agent against the refractory, resistant, or non-responsive cancer. Administration of the combination of the CD44-modulating polypeptide and anti-cancer agent(s) surprisingly can restore or enhance the activity of the CD44-modulating polypeptide against the refractory, resistant, or non-responsive cancer. Administration of the combination of the CD44-modulating polypeptide and anti-cancer agent(s) surprisingly can restore or enhance the activity of the CD44-modulating polypeptide and the anti-cancer agent against the refractory, resistant, or non-responsive cancer.

In one embodiment of methods of treating described herein, a patient can be administered a combination of a CD44-modulating polypeptide described herein and a radiation therapy where the cancer treated is resistant, refractory, or non-responsive to one of or both the CD44-modulating polypeptide and the radiation therapy. In one example, the cancer can be resistant, refractory, or non-responsive to treatment with radiation therapy. Administration of the combination of the CD44-modulating polypeptide and anti-cancer agent(s) surprisingly can restore or enhance the activity of the radiation therapy against the refractory, resistant, or non-responsive cancer. Administration of the combination of the CD44-modulating polypeptide and anti-cancer agent(s) surprisingly can restore or enhance the activity of the CD44-modulating polypeptide against the refractory, resistant, or non-responsive cancer. Administration of the combination of the CD44-modulating polypeptide and radiation therapy surprisingly can restore or enhance the activity of the CD44-modulating polypeptide and the radiation therapy against the refractory, resistant, or non-responsive cancer.

In one example, a CD44-modulating polypeptide described herein does not have activity against a cancer described herein when administered alone. In one embodiment, where a CD44-modulating polypeptide described herein does not have activity against a cancer described herein when administered alone, its activity can be established or restored when administered in combination with an anti-cancer agent described herein, a radiation therapy described herein, or a combination thereof. In another example, a CD44-modulating polypeptide described herein has minimal activity against a cancer described herein (e.g., insufficient anti-cancer activity to treat a cancer described herein) when administered alone. In one embodiment, where a CD44-modulating polypeptide described herein has minimal activity against a cancer described herein, its activity can be enhanced when administered in combination with an anti-cancer agent described herein, a radiation therapy described herein, or a combination thereof.

In another example, an anti-cancer agent described herein or a radiation therapy described herein does not have activity against a cancer described herein when administered alone (or in combination with another anti-cancer agent). In one embodiment, where an anti-cancer agent described herein or a radiation therapy described herein does not have activity against a cancer described herein when administered alone, its activity can be restored when administered in combination with CD44-modulating polypeptide described herein. In another example, an anti-cancer agent described herein or a radiation therapy described herein has minimal activity against, a cancer described herein (e.g., insufficient anti-cancer activity to treat, a cancer described herein) when administered alone. In one embodiment, where an anti-cancer agent described herein or a radiation therapy described herein has minimal activity against a cancer described herein, its activity can be enhanced when administered in combination with a CD44-modulating polypeptide described herein. In another example, an anti-cancer agent or a radiation therapy can lose its anti-cancer activity over the course of treatment due to, for example, progression of resistance or refraction in the cancer treated. In one embodiment, the loss of anti-cancer agent or a radiation therapy activity can be slowed, stopped, or reversed (e.g., enhanced activity) when the patient is administered the anti-cancer agent or a radiation therapy in combination with a CD44-modulating polypeptide described herein.

In certain instances, the methods above include administration of a polypeptide comprising or consisting of SEQ ID NO:1 or SEQ ID NO:2. In one embodiment, the methods described herein include administration of a polypeptide of SEQ ID NO:1. In another embodiment, the methods described herein include administration of a polypeptide of SEQ ID NO:1, wherein the polypeptide includes mutation of K¹ as described above. In another embodiment, the methods described herein include administration of a polypeptide of SEQ ID NO:1 where the polypeptide includes a mutation of P², P⁵, P⁶, or a combination thereof as described above. In another embodiment the methods described herein include; administration of a polypeptide of SEQ ID NO:1 where the polypeptide includes a mutation of S³, S⁴, or a combination thereof as described above. In another embodiment the methods described herein include administration of a polypeptide of SEQ ID NO:1 where the polypeptide includes a mutation of E⁷, E⁸ or a combination thereof as described above.

In certain instances, treating a patient comprises administration of a CD44-modulating polypeptide in combination with a radiation therapy. A variety of radiation therapies are available, which may be advantageously utilized in accordance with the present disclosure. Radiation may come from a machine outside the body (external radiation), may be placed inside the body (internal radiation, e.g. brachytherapy), or may use unsealed radioactive materials that go throughout the body (systemic radiation therapy). The type of radiation to be given depends on the type of cancer, its location, how far into the body the radiation will need to penetrate, the patient's general health and medical history, whether the patient will have other types of cancer treatment, and other factors. In certain embodiments, radiation is delivered in more than one manner, e.g., internal radiation and external radiation. Radiation therapy may be intraoperative radiation therapy, which may, for example, be administered at one or more stages during a surgical procedure. Typically, radiation therapy comprises exposure to ionizing radiation, non-limiting examples of which include X-Rays, gamma-Rays, UV-Rays, particle beams, and decay of radioactive isotopes. Examples of particle beams include, without limitation, neutron beams, proton beams, carbon ion beams, and pion beams.

In one example radiation therapy procedure, a beam of high-energy X-ray's, generated outside the subject by a linear accelerator, is delivered to a tumor. When the cancer tumor is within the path of the X-ray, it receives some of that radiation; however, surrounding healthy tissue receives radiation as well. In order to limit the extent of collateral tissue damage, the tumor area is typically bombard with the lowest level of effective radiation from many different points of entrance in an attempt to minimize damage to normal tissues.

In some embodiments, the radiation therapy is stereotactic (or stereotaxic) radiotherapy which uses a large dose of radiation to destroy tumor tissue. In certain cases, where the cancer is in the brain, the patient's head can be placed in a frame, which is attached or is fitted to the patient's skull. The frame is used to aim high-dose radiation beams directly at the tumor inside the patient's head. The dose and area receiving the radiation are coordinated precisely resulting in little damage to nearby' tissues. In some stereotactic applications, a head frame is not used. In certain embodiments, real-time imaging systems are used in conjunction with the movement of an accelerator, allowing computer adjustments of the accelerator trajectory to compensate for any motion of the patient's head. A radiation dose administered by stereotactic radiotherapy may be administered over multiple fractions to reduce toxicity to normal tissue. A further example of radiation therapy procedure is stereotactic radiosurgery, which refers to a non-surgical procedure that delivers a high-dose of precisely-targeted radiation, such as targeted to the brain, head, or neck, using highly focused beams of radiation (e.g. gamma-rays or x-ray's) that converge on the specific area or areas where the tumor resides, minimizing the amount of radiation to healthy tissue. Stereotactic radiosurgery may be done in a variety of ways. One suitable technique uses a linear accelerator to administer high-energy photon radiation to the tumor. In another technique, a gamma knife uses cobalt 60 to deliver radiation. In a third technique, heavy charged particle beams such as protons and helium ions are used to deliver stereotactic radiation to the tumor.

In some embodiments, the patient is subjected to radiation therapy following resection of cancerous cells, or may even be subjected to radiation both prior to and following resection of cancerous cells. In some embodiments, the patient undergoes surgical resection of a tumor and radiation therapy is administered to the patient during the surgical procedure, following removal of the tumor, or both during the surgical procedure and following the removal of the tumor.

The energy source used for the radiation therapy may be selected from X-rays or gamma rays, which are both forms of electromagnetic radiation. X-rays can be created by machines called linear accelerators. Depending on the amount of energy the x-rays have, they can be used to destroy cancer cells on the surface of the body (in the case of lower energy rays), or deeper into tissues and organs (in the case of higher energy rays). Compared with other types of radiation, x-rays can deliver radiation to a relatively large area. Gamma rays can be produced by isotopes of certain elements, such as iridium and cobalt 60, which release radiation energy as they decay. Each element decays at a specific rate and each gives off a different amount of energy, which affects how deeply it can penetrate into the body. Gamma rays produced by the decay of cobalt 60 are used in the treatment referred to as the “gamma knife.”

The energy source for the radiation therapy may be selected from particle beams, which typically use fast-moving subatomic particles instead of photons. This type of radiation may be referred to as particle beam radiation therapy or particulate radiation. Particle beams may be created by linear accelerators, synchrotrons, betatrons and cyclotrons, which produce and accelerate the particles. Particle beam therapy may use electrons, which can be produced by an x-ray tube; neutrons, which can be produced by radioactive elements and special equipment; heavy ions such as protons, carbon ions and helium; and/or pi-mesons, also called pions, which are small, negatively charged particles produced by an accelerator and a system of magnets. Unlike x-rays and gamma rays, some particle beams, depending on the energy, can penetrate only a short distance into tissue. Therefore, they may be used to treat cancers located on the surface of or just below the skin.

In some embodiments, radioactive isotopes are the source of ionizing radiation for radiation therapy. Non-limiting examples of radioactive isotopes include radioactive isotopes of iodine (e.g. iodine 125 or iodine 131), strontium, phosphorous, palladium, cesium, iridium, phosphate, samarium, yttrium, or cobalt (e.g. cobalt 60). In particular, iodine 125 (t 1/2=60.1 days), palladium 103 (t 1/2=17 days), cesium 137, strontium 89 (t 1/2-50.5 days), samarium 153 (t 1/2=46.3 hours), and iridium 192, can advantageously be used. Radiation may be delivered directly to the cancer through the use of radiolabeled antibodies, also referred to as radioimmunotherapy. This approach can reduce or minimize the risk of radiation damage to healthy cells. In certain embodiments, the radioimmunotherapy treatments are selected from ibritumomab tiuxetan (Zevalin®) and tositumomab and iodine 131 tositumomab (Bexxar®). Radioimmunotherapy may be used in the treatment of advanced adult non-Hodgkin lymphoma (NHL). In certain embodiments, immunotherapy is used in the treatment of cancers including leukemia, NHL, colorectal cancer, and cancers of the liver, lung, brain, prostate, thyroid, breast, ovary, and pancreas.

In some embodiments, the patient is subjected to more than one form of radiation therapy, such as two or more forms of radiation therapy at the same time, in sequence, in fractional doses at the same time or in fractional doses sequentially, in fractional doses alternating, and/or any combination thereof. In certain embodiments, intraoperative radiation therapy is administered before, during and/or after a surgical procedure and a second form or radiation therapy is administered at a later time such as hours after the surgical procedure, and/or days after the surgical procedure, and/or weeks after the surgical procedure. In certain embodiments, the patient is treated with radiation therapy leading up to the surgical procedure such as hours before the surgical procedure, days before the surgical procedure and/or weeks before the surgical procedure.

In one embodiment, the combination(s) of a CD44-modulating polypeptide described herein and a radiation therapy described herein can enhance the activity of the radiation therapy in treating cancer, where the cancer is either not resistant to the radiation therapy or is resistant to the radiation therapy. In another embodiment, the combination(s) above can restore the anti-cancer activity of the radiation therapy in treating a radiation therapy-resistant cancer. In another embodiment, the combinations above can slow or prevent the onset of radiation therapy resistance in a cancer during treatment. In some embodiments, combination of the CD44-modulating polypeptide and the radiation therapy reduces the therapeutically effective amount of the CD44-modulating polypeptide, the radiation therapy, or both relative to the therapeutically effective amount when used alone.

In certain instances, treating a patient comprises administration of a CD44-modulating polypeptide in combination with a radiation therapy. The anti-cancer agent used in combination with a polypeptide described herein can be a taxane, a platinum agent, anthracyclin, or a checkpoint inhibitor. The anti-cancer agent can be taxane. The anti-cancer agent can be taxol, taxotere, paclitaxel, or cabazitaxel. The cancer treated can be resistant, refractory, or non-responsive to taxane treatment. For example, the cancer can be taxane resistant ovarian cancer, breast cancer, or prostate cancer. In one embodiment, the anti-cancer agent is not paclitaxel. In still another embodiment, the anti-cancer agent can be a drug other than paclitaxel. In certain instances the combination administered to a patient described herein contains a taxane other than paclitaxel in combination with a CD44-modulating polypeptide described herein. In certain instances the combination administered to a patient described herein contains a taxane other than paclitaxel in combination with a CD44-modulating polypeptide described herein and a radiation therapy described herein. In one embodiment, the combination includes paclitaxel and a polypeptide of SEQ ID NO:1; paclitaxel and a polypeptide of SEQ ID NO:2; taxol and a polypeptide of SEQ ID NO:1; taxol and a polypeptide of SEQ ID NO:2; taxotere and a polypeptide of SEQ ID NO:1, taxotere and a polypeptide of SEQ ID NO:2; cabazitaxel and a polypeptide of SEQ ID NO:1; or cabazitaxel and a polypeptide of SEQ ID NO:2. Such combinations can further include administration of one or more radiation therapies described herein. In one example the combination includes paclitaxel in combination with a polypeptide of SEQ ID NO:1 or SEQ ID NO:2.

The combination of a taxane and CD44-modulating polypeptide (e.g. SEQ ID NO:1 or SEQ ID NO:2) can restore or enhance the anti-cancer activity of the taxane against the cancer. In one example, the combination of taxol and a polypeptide of SEQ ID NO:1; taxol and a polypeptide of SEQ ID NO:2; taxotere and a polypeptide of SEQ ID NO:1, taxotere and a polypeptide of SEQ ID NO:2; cabazitaxel and a polypeptide of SEQ ID NO:1; or cabazitaxel and a polypeptide of SEQ ID NO:2 can restore or enhance the activity of taxol, taxotere, and/or cabazitaxel in treating taxane resistant cancer. Such combinations can further include administration of one or more radiation therapies described herein. In one embodiment, the combination(s) above can enhance the activity of the taxane in treating cancer, where the cancer is either not taxane resistant or is taxane resistant. In another embodiment, the combination(s) above can restore the anti-cancer activity of the taxane in treating a taxane resistant cancer. In another embodiment, the combinations above can slow or prevent the onset of taxane resistance in a cancer during treatment. The taxane resistant cancer can be breast cancer, ovarian cancer, or prostate cancer.

The anti-cancer agent can be a platinum agent such as, for example, cisplatin, carboplatin, oxaliplatin, satraplatin, picoplatin, nedaplatin, or triplatin. In certain instance the platinum agent can be carboplatin or oxaliplatin. The cancer treated can be resistant to platinum agent treatment. For example, the platinum agent resistant cancer can be breast cancer or ovarian cancer. In one embodiment, the platinum agent is not cisplatin. In another embodiment, the anti-cancer agent can be a drug other than cisplatin. In one embodiment, the combination can include carboplatin and a polypeptide of SEQ ID NO:1; carboplatin and a polypeptide of SEQ ID NO:2; oxaliplatin and a polypeptide of SEQ ID NO:1, oxaliplatin and a polypeptide of SEQ ID NO:2; satraplatin and a polypeptide of SEQ ID NO:1, satraplatin and a polypeptide of SEQ ID NO:2; picoplatin and a polypeptide of SEQ ID NO:1; picoplatin and a polypeptide of SEQ ID NO:2; triplatin and a polypeptide of SEQ ID NO:1, triplatin and a polypeptide of SEQ ID NO:2; nedaplatin and a polypeptide of SEQ ID NO:1; or nedaplatin and a polypeptide of SEQ ID NO:2. In another example, the combination can include cisplatin in combination with a polypeptide of SEQ ID NO:1 or a polypeptide of SEQ ID NO:2. It is to be understood that the embodiments described herein can further include administration one or more radiation therapies as described herein.

The combination of a platinum agent and CD44-modulating polypeptide (e.g. SEQ ID NO:1 or SEQ ID NO:2) can restore or enhance the anti-cancer activity of the platinum agent against the cancer. In one example, the combination of carboplatin and a polypeptide of SEQ ID NO:1; carboplatin and a polypeptide of SEQ ID NO:2; oxaliplatin and a polypeptide of SEQ ID NO:1, oxaliplatin and a polypeptide of SEQ ID NO:2; satraplatin and a polypeptide of SEQ ID NO:1, satraplatin and SEQ ID NO:2; picoplatin and a polypeptide of SEQ ID NO:1; picoplatin and a polypeptide of SEQ ID NO:2; triplatin and a polypeptide of SEQ ID NO:1, triplatin and a polypeptide of SEQ ID NO:2; nedaplatin and a polypeptide of SEQ ID NO:1; or nedaplatin and a polypeptide of SEQ ID NO:2 can restore or enhance the activity of carboplatin, oxaliplatin, satraplatin, picoplatin, nedaplatin, or triplatin in treating taxane resistant cancer. It is to be understood that the embodiments described herein can further include administration one or more radiation therapies as described herein.

In one embodiment, the combination(s) above can enhance the activity of the platinum agent in treating cancer, where the cancer is either not platinum agent resistant or is platinum agent resistant. In another embodiment, the combination(s) above can restore the anti-cancer activity of the platinum agent in treating a platinum agent resistant cancer. In another embodiment, the combinations above can slow or prevent the onset of platinum agent resistance in a cancer during treatment. The platinum agent resistant cancer can be breast cancer or ovarian cancer. It is to be understood that the embodiments described herein can further include administration one or more radiation therapies as described herein.

The anti-cancer agent can be paclitaxel, a paclitaxel analogue, docetaxel, cabaziltaxel, doxorubicin, a checkpoint inhibitor, cisplatin, oxaliplatin, carboplatin, methotrexate, or a PARP inhibitor. In one embodiment, the anti-cancer agent can be methotrexate. In certain embodiments, the anti-cancer agent can be paclitaxel, a paclitaxel analogue, docetaxel, cabaziltaxel, doxorubicin, a checkpoint inhibitor, cisplatin, oxaliplatin carboplatin, methotrexate, or a PARP inhibitor which can be administered in combination with a polypeptide of SEQ ID NO:1 or a polypeptide of SEQ ID NO:2. It is to be understood that the embodiments described herein can further include administration one or more radiation therapies as described herein.

In another example the anti-cancer agent can be methotrexate. The cancer treated can be resistant, refractory, or non-responsive to methotrexate treatment. For example, the cancer can be methotrexate resistant ovarian cancer, breast cancer, or colorectal cancer. In one embodiment, a combination of methotrexate and a polypeptide of SEQ ID NO:1 or methotrexate and a polypeptide of SEQ ID NO:2 can be used in the methods of treating described herein. In one embodiment, a combination of methotrexate, a polypeptide of SEQ ID NO:1, and a radiation therapy described herein; or methotrexate, a polypeptide of SEQ ID NO:2, and a radiation therapy described herein can be used in the methods of treating described herein.

The combination of methotrexate and CD44-modulating polypeptide (e.g. SEQ ID NO:1 or SEQ ID NO:2) can restore or enhance the anti-cancer activity of methotrexate against the cancer. In one example, the combination of methotrexate and a polypeptide of SEQ ID NO:1 and methotrexate and a polypeptide of SEQ ID NO:2 can restore or enhance the activity of methotrexate in treating methotrexate resistant cancer. In one embodiment, the combination(s) above can enhance the activity of methotrexate in treating cancer, where the cancer is either not methotrexate resistant or is methotrexate resistant. In another embodiment, the combination(s) above can restore the anti-cancer activity of methotrexate in treating a methotrexate resistant cancer. In another embodiment, the combinations above can slow or prevent the onset of methotrexate resistance in a cancer during treatment. The methotrexate resistant cancer can be breast cancer, ovarian cancer, or colon cancer.

The anti-cancer agent can be an anthracyclin agent. The anti-cancer agent can be amrubicin, daunorubicin, epirubicin, idarubicin, doxorubicin, pirarubicin, or valrubicin. The cancer treated can be resistant to treatment with an anthracyclin agent. For example, the anti-cancer agent can be amrubicin, daunorubicin, epirubicin, idarubicin, doxorubicin, pirarubicin, or valrubicin. The cancer can be a cancer that is or develops resistance, refraction, or non-responsiveness one or more of the agents. In one example, the cancer can be breast cancer or ovarian cancer that is resistant, refractory or non-responsive to treatment with one or more anthracyclin agents. In another example, the cancer can be breast cancer that is resistant, refractory, or non-responsive to treatment with one or more anthracyclin agents and one or more taxanes (e.g. taxol or paclitaxel). It is to be understood that the embodiments described herein can further include administration one or more radiation therapies as described herein.

The combination of an anthracyclin agent and CD44-modulating polypeptide (e.g. SEQ ID NO:1 or SEQ ID NO:2) can restore or enhance the anti-cancer activity of the anthracyclin agent against the cancer. In one example, the combination of amrubicin and a polypeptide of SEQ ID NO:1; amrubicin and a polypeptide of SEQ ID NO:2; daunorubicin and a polypeptide of SEQ ID NO:1, daunorubicin and a polypeptide of SEQ ID NO:2; epirubicin and a polypeptide of SEQ ID NO:1; epirubicin and a polypeptide of SEQ ID NO:2; idarubicin and a polypeptide of SEQ ID NO:1, idarubicin and a polypeptide of SEQ ID NO:2; doxorubicin and a polypeptide of SEQ ID NO:1; doxorubicin and a polypeptide of SEQ ID NO:2; pirarubicin and a polypeptide of SEQ ID NO:1; or pirarubicin and a polypeptide of SEQ ID NO:2 valrubicin and a polypeptide of SEQ ID NO:1; or valrubicin and a polypeptide of SEQ ID NO:2 can restore or enhance the activity of amrubicin, daunorubicin, epirubicin, idarubicin, doxorubicin, pirarubicin, or valrubicin in treating anthracyclin resistant cancer. In another example, the combination of amrubicin and a polypeptide of SEQ ID NO:1; amrubicin and a polypeptide of SEQ ID NO:2; daunorubicin and a polypeptide of SEQ ID NO:1, daunorubicin and a polypeptide of SEQ ID NO:2; epirubicin and a polypeptide of SEQ ID NO:1; epirubicin and a polypeptide of SEQ ID NO:2; idarubicin and a polypeptide of SEQ ID NO:1, idarubicin and a polypeptide of SEQ ID NO:2; doxorubicin and a polypeptide of SEQ ID NO:1; doxorubicin and a polypeptide of SEQ ID NO:2; pirarubicin and a polypeptide of SEQ ID NO:1; or pirarubicin and a polypeptide of SEQ ID NO:2 valrubicin and a polypeptide of SEQ ID NO:1; or valrubicin and a polypeptide of SEQ ID NO:2 can restore or enhance the activity of amrubicin, daunorubicin, epirubicin, idarubicin, doxorubicin, pirarubicin, or valrubicin in treating anthracyclin and taxane resistant cancer. It is to be understood that the embodiments described herein can further include administration one or more radiation therapies as described herein.

The anti-cancer agent can be a PARP inhibitor. The PARP inhibitor can inhibit one or both of PARP1 and PARP2. The PARP inhibitor can be talazoparib (BMN 673), olaparib, rucaparib, iniparib, or veliparib. The cancer treated can be resistant, refractory, or non-responsive to PARP inhibitor treatment. For example, the cancer can be PARP inhibitor resistant ovarian cancer, breast cancer, pancreatic, or prostate cancer. In one embodiment, the combination includes talazoparib and a polypeptide of SEQ ID NO:1; talazoparib and a polypeptide of SEQ ID NO:2; olaparib and a polypeptide of SEQ ID NO:1 , olaparib and a polypeptide of SEQ ID NO:2; rucaparib and a polypeptide of SEQ ID NO:1, rucaparib and a polypeptide of SEQ ID NO:2; iniparib and a polypeptide of SEQ ID NO:1, iniparib and a polypeptide of SEQ ID NO:2; veliparib and a polypeptide of SEQ ID NO:1; or veliparib and a polypeptide of SEQ ID NO:2. It is to be understood that, the embodiments described herein can further include administration one or more radiation therapies as described herein. The PARP inhibitor resistant cancer can be breast cancer, ovarian cancer, pancreatic, or prostate cancer.

The combination of a PARP inhibitor and CD44-modulating polypeptide (e.g. SEQ ID NO:1 or SEQ ID NO:2) can restore or enhance the anti-cancer activity of the PARP inhibitor against the cancer. In one example, the combination of talazoparib and a polypeptide of SEQ ID NO:1; talazoparib and a polypeptide of SEQ ID NO:2; olaparib and a polypeptide of SEQ ID NO:1, olaparib and a polypeptide of SEQ ID NO:2; rucaparib and a polypeptide of SEQ ID NO:1, rucaparib and a polypeptide of SEQ ID NO:2; iniparib and a polypeptide of SEQ ID NO:1, iniparib and a polypeptide of SEQ ID NO:2; veliparib and a polypeptide of SEQ ID NO:1; or veliparib and a polypeptide of SEQ ID NO:2 can restore or enhance the activity of talazoparib (BMN 673), olaparib, rucaparib, iniparib, or veliparib in treating PARP inhibitor resistant cancer. In one embodiment, the combination(s) above can enhance the activity of the PARP inhibitor in treating cancer, where the cancer is either not PARP inhibitor resistant or is PARP inhibitor resistant. In another embodiment, the combination(s) above can restore the anti-cancer activity of the taxane in treating a taxane resistant cancer. In another embodiment, the combinations above can slow or prevent the onset of PARP inhibitor resistance in a cancer during treatment. The PARP inhibitor resistant cancer can be breast cancer, ovarian cancer, pancreatic, or prostate cancer. It is to be understood that the embodiments described herein can further include administration one or more radiation therapies as described herein.

In certain instances, the CD44-modulating polypeptide does not have anti-cancer activity against a cancer described herein when administered alone. In one example, a polypeptide of SEQ ID NO:1 or a polypeptide of SEQ ID NO:2 does not have anti-cancer activity against a cancer described herein when administered alone. In one embodiment, such a polypeptide not having anti-cancer activity can restore or enhance the activity of a co-administered anti-cancer agent described herein. In one embodiment, such a polypeptide not having anti-cancer activity can restore or enhance the activity of a co-administered radiation therapy as described herein. In another embodiment, such a polypeptide not having anti-cancer activity can, in the presence of other anti-cancer agents, have anti-cancer activity. In another embodiment, such a polypeptide not having anti-cancer activity can, in the presence of radiation therapy, have anti-cancer activity.

In one embodiment, coadministration of a polypeptide of SEQ ID NO:1 or a polypeptide of SEQ ID NO:2, having known inactivity against a specific cancer, with an anti-cancer agent restores or enhances the anti-cancer activity of the polypeptide of SEQ ID NO:1 or a polypeptide of SEQ ID NO:2. In one embodiment, coadministration of a polypeptide of SEQ ID NO:1 or a polypeptide of SEQ ID NO:2, having known inactivity against a specific cancer, with radiation therapy restores or enhances the anti-cancer activity of the polypeptide of SEQ ID NO:1 or a polypeptide of SEQ ID NO:2. In another embodiment, coadministration of a polypeptide of SEQ ID NO:1 or a polypeptide of SEQ ID NO:2, having known inactivity against a specific cancer, with an anti-cancer agent restores or enhances the anti-cancer activity of the polypeptide of SEQ ID NO:1 or a polypeptide of SEQ ID NO:2. In still another embodiment, coadministration of a polypeptide of SEQ ID NO:1 or a polypeptide of SEQ ID NO:2, having known inactivity against a specific cancer, with radiation therapy restores or enhances the anti-cancer activity of the polypeptide of SEQ ID NO:1 or a polypeptide of SEQ ID NO:2.

In another embodiment, coadministration of a polypeptide of SEQ ID NO:1 or SEQ ID NO:2, having known inactivity against a specific cancer, with an anti-cancer agent to a patient having a cancer with resistance to the cancer agent, restores or enhances the anti-cancer activity of the polypeptide of SEQ ID NO:1 or SEQ ID NO:2 and/or the anti-cancer agent. In another embodiment, coadministration of a polypeptide of SEQ ID NO:1 or SEQ ID NO:2, having known inactivity against a specific cancer, with radiation therapy to a patient having a cancer with resistance to the radiation therapy, restores or enhances the anti-cancer activity of the polypeptide of SEQ ID NO:1 or SEQ ID NO:2 and/or the radiation therapy. In one embodiment, the combination of CD44-modulating polypeptide described herein with an anti-cancer agent described herein is useful in the treatment of cancers resistant to one or more anti-cancer agents and/or a CD44-modulating polypeptide. In still another embodiment, the combination of CD44-modulating polypeptide described herein with radiation therapy described herein is useful in the treatment of cancers resistant to radiation therapy and/or a CD44-modulating polypeptide.

While exemplified above, the anti-cancer agent useful in the methods provided herein can include any known class of anti-cancer agents such as, for example, operations, alkylating agents, antimetabolites, anthracyclines, campothecins, vinca alkaloids, taxanes or platinums, as well as other antineoplastic agents known in the art. Such anti-cancer agent and antineoplastic agent classifications are known in the art and used in accordance with their plain and ordinary meaning. Such anti-cancer agents and anti-cancer agent classes can in certain instances can provide anti-cancer activity in combination with each other. In one example, two or more different classes of anti-cancer agents described herein can be administered in combination and together with a CD44-modulating polypeptide described herein. In another example, one or more different classes of anti-cancer agents described herein can be administered in combination and together with a CD44-modulating polypeptide described herein and a radiation therapy as described herein. The CD44-modulating polypeptide can be a CD44-modulating polypeptide described herein.

In another example, two or more anti-cancer agents of the same class can be administered in combination with a CD44-modulating polypeptide described herein. In still another example, two or more anti-cancer agents of the same class can be administered in combination with a CD44-modulating polypeptide described herein and a radiation therapy described herein. Combination of anti-cancer agents described herein can be performed in accordance with a package insert. The term package insert refers to instructions customarily included in commercial packages of medicaments approved by the FDA or a similar regulatory agency of a country other than the USA, which contains information about, for example, the usage, dosage, administration, contraindications, and/or warnings concerning the use of such medicaments. Various examples of combinations of CD44-modulating polypeptides and one or more anti-cancer agents are provided below, and it is expressly contemplated that such combinations may further comprise combination with a radiation therapy.

In certain instances, the methods employ a CD44-modulating polypeptide described herein in combination with radiation therapy for treating a cancer described herein. Radiation therapy useful in the methods described herein can also include administration of an anti-cancer agent described herein. The methods of treating cancer described herein can include operations for treating or resecting cancer from a patient in combination with the polypeptide of SEQ ID NO:1 or SEQ ID NO:2. In another example the patient has undergone at least one surgery to treat the cancer.

Exemplary anti-cancer agents useful in the methods described herein include but are not limited to: ABRAXANE; abiraterone; ace-11; aclarubicin; acivicin; acodazole hydrochloride; acronine; actinomycin; acylfulvene; adecypenol; adozelesin; adriamycin; aldesleukin; all trans-retinoic acid (ATRA); altretamine; ambamustine; ambomycin; ametantrone acetate; amidox; amifostine; aminoglutethimide; aminolevulinic acid; amrubicin; amsacrine; anagrelide; anastrozole; andrographolide; antarelix; anthramycin; aphidicolin glycinate; apurinic acid; ara-CDP-DL-PTBA; arginine deaminase; ARRY-162; ARRY-300; ARRY-142266; AS703026; asparaginase; asperlin; asulacrine; atamestane; atrimustine; AVASTIN; axinastatin 1; axinastatin 2; axinastatin 3; azasetron; azatoxin; azatyrosine; azacitidine; AZD8330; azetepa; azotomycin; balanol; batimastat; BAY 11-7082; BAY 43-9006; BAY 869766; bendamustine; benzochlorins; benzodepa; benzoylstaurosporine; beta-alethine; betaclamycin B; betulinic acid; b-FGF inhibitor; bicalutamide; bisantrene; bisaziridinylspermine; bisnafide; bisnafide dimesylate; bistratene A; bisantrene hydrochloride; bleomycin; bleomycin sulfate; busulfan; bizelesin; breflate; bortezomib; brequinar sodium; bropirimine; budotitane; buthionine sulfoximine; bryostatin; cactinomycin; calusterone; calcipotriol; calphostin C; camptothecin derivatives; capecitabine; carboxamide-amino-triazole; carboxyamidotriazole; CaRest M3; CARN 700; caracemide; carbetimer; carboplatin; carmustine; carubicin hydrochloride; carzelesin; castanospermine; cecropin B; cedefingol; celecoxib; cetrorelix; chlorins; chloroquinoxaline sulfonamide; cicaprost; chlorambucil; Chlorofusin; cirolemycin; cisplatin; CI-1040; cis-porphyrin; cladribine; clomifene analogues; clotrimazole; collismycin A; collismycin B; combretastatin A4; combretastatin analogue; conagenin; crambescidin 816; crisnatol; crisnatol mesylate; cryptophycin 8; cryptophycin A derivatives; curacin A; cyclopentanthraquinones; cycloplatam; cypemycin; cyclophosphamide; cytarabine; cytarabine ocfosfate; cytolytic factor; cytostatin; dacarbazine; dactinomycin; daunorubicin; daunorubicin hydrochloride; dacarbazine; dacliximab; dasatinib; decitabine; dehydrodidemnin B: deslorelin; dexamethasone; dexifosfamide; dexrazoxane; dexverapamil; dexormaplatin; dezaguanine; dezaguanine mesylate; diaziquone; didemnin B; didox; diethylnorspermine; dihydro 5 azacytidine; dihydrotaxol; 9-dioxamycin; diphenyl spiromustine; docosanol; dolasetron; docetaxel; doxorubicin; doxorubicin hydrochloride; doxifluridine; droloxifene; droloxifene citrate; dromostanolone propionate; dronabinol; duazomycin; duocarmycin SA; ebselen; ecomustine; edelfosine; edrecolomab; edatrexate; eflornithine hydrochloride; eflornithine; elemene; emitefur; elsamitrucin; enloplatin; enpromate; epipropidine; epirubicin; epirubicin hydrochloride; epristeride; erbulozole; eribulin; esorubicin hydrochloride; estramustine; estramustine phosphate sodium; etanidazole; etoposide; etoposide phosphate; etoprine; exemestane; fadrozole; fadrozole hydrochloride; fazarabine; fenretinide; filgrastim; finasteride; flavopiridol; flezelastine; fluasterone; floxuridine; fludarabine phosphate; fludarabine; fluorodaunorubicin hydrochloride; forfenimex; formestane; fluorouracil; floxouridine; flurocitabine; fosquidone; fostriecin sodium; fostriecin; fotemustine; gadolinium texaphyrin; gallium nitrate; galocitabine; ganirelix; gelatinase inhibitors; gemcitabine; geldanamycin; gossyphol; GDC-0973; GSK1120212/trametinib; herceptin; hydroxyurea; hepsulfam; heregulin; hexamethylene bisacetamide; hypericin; ibandronic acid; ibrutinib; idarubicin; idarubicin hydrochloride; ifosfamide; canfosfamide; ilmofosine; iproplatin; idoxifene; idramantone; ilmofosine; ilomastat; imidazoacridones; imatinib (e.g., GLEEVEC); imiquimod; iniparib (BSI 201); iobenguane; iododoxorubicin; ipomeanol; irinotecan; irinotecan hydrochloride; irsogladine; isobengazole; isohomohalicondrin B; itasetron; iimofosine; interleukin IL-2 (including recombinant interleukin II; or rIL.sub.2); interferon alfa-2a; interferon alfa-2b; interferon alfa-n1; interferon alfa-n3; interferon beta-1a; interferon gamma-1b; jasplakinolide; kahalalide F; lamellarin N triacetate; lanreotide; leinamycin; lenograstim; lentinan sulfate; leptolstatin; letrozole; leuprorelin; levamisole; lenalidomide; lenvatinib; liarozole; lissoclinamide 7; lobaplatin; lombricine; lometrexol; lonidamine; losoxantrone; lovastatin; loxoribine; lurtotecan; lutetium texaphyrin; lysofylline; lanreotide acetate; lapatinib; letrozole; leucovorin; leuprolide acetate; liarozole hydrochloride; lometrexol sodium; lomustine; losoxantrone hydrochloride; pomalidomide; LY294002; maitansine; mannostatin A; marimastat; masoprocol; maspin; matrilysin inhibitors; menogaril; merbarone; meterelin; methioninase; metoclopramide; MIF inhibitor; mifepristone; miltefosine; mirimostim; mitoguazone; mitolactol; mitonafide; mitoxantrone; mofarotene; molgramostim; mopidamol; mycaperoxide B; myriaporone; maytansine; mechlorethamine hydrochloride; megestrol acetate; melengestrol acetate; melphalan; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nafarelin; nagrestip; napavin; naphterpin; nartograstim; nedaplatin; nemorubicin; neridronic acid; nilutamide; nisamycin; nitric oxide modulators; nitroxide antioxidant; nitrullyn; nocodazole; oblimersen (GENASENSE); octreotide; okicenone; olaparib (LYNPARZA); oligonucleotides; onapristone; ondansetron; oracin; oral cytokine inducer; ormaplatin; oxisuran; oxaloplatin; osaterone; oxaliplatin; oxaunomycin; palauamine; palmitoylrhizoxin; pamidronic acid; panaxytriol; panomifene; parabactin; PAMP (polyADP ribose polymerase) inhibitors; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; porfiromycin; prednisone; prostaglandin J2; pyrazoloacridine; paclitaxel; PD035901; PD184352; PD318026; PD98059; peliomycin; pentamustine; peplomycin sulfate; PKC412; pipobroman; piposulfan; piroxantrone hydrochloride; plicamycin; plomestane; podophyllotoxin; polyphenol E; porfimer sodium; porfiromycin; prednimustine; procarbazine; procarbazine hydrochloride; puromycin; puromycin hydrochloride; pyrazofurin; raltitrexed; ramosetron; retelliptine demethylated; rhizoxin; rituximab; RII retinamide; rogletimide; rohitukine; romurtide; roquinimex; rubiginone B1; ruboxyl; riboprine; romidepsin; rucaparib; safingol; safingol hydrochloride; saintopin; sarcophytol A; sargramostim; semustine; sizofiran; sobuzoxane; sodium borocaptate; sodium phenylacetate; solverol; sonennin; soratenib; sunitinib; sparfosic acid; spicamycin D; spiromustine; splenopentin; spongistatin 1; Spongistatin 2; Spongistatin 3; Spongistatin 4; Spongistatin 5; Spongistatin 6; Spongistatin 7; Spongistatin 8; and Spongistatin 9; squalamine; stipiamide; stromelysin inhibitors; sulfinosine; suradista; suramin; swainsonine; SB239063; selumetinib/AZD6244; simtrazene; SP600125; sparfosate sodium; sparsomycin; spirogertnanium hydrochloride; spiroplatin; streptonigrin; streptozocin; sulofenur; tallimustine; tamoxifen methiodide; talazoparib (BMN 673); tauromustine; tazarotene; tecogalan sodium; tegafur; tellurapyrylium; temoporfin; temozolomide; teniposide; tetrachlorodecaoxide; tetrazomine; thaliblastine; thiocoraline; thrombopoietin; thymalfasin; thymopoietin receptor agonist; thymotrinan; tirapazamine; titanocene bichloride; topsentin; toremifene; tretinoin; triacetyluridine; triciribine; trimetrexate; triptorelin; tropisetron; turosteride; tyrphostins; talisomycin; TAK-733; taxotere; tegafur; teloxantrone hydrochloride; teroxirone; testolactone; thiamiprine; thioguanine; thiotepa; tiazofurin; tirapazamine; toremifene citrate; trastuzurmab; trestolone acetate; triciribine phosphate; trimetrexate; trimetrexate glucuronate; triptorelin; tubulozole hydrochloride; tumor necrosis factor-related apoptosis-inducing ligand (TRAIL); UBC inhibitors; ubenimex; U0126; uracil mustard; uredepa; vapreotide; variolin B; velaresol; veliparib (ABT-888); veramine; verteporfin; vinorelbine; vinxaltine; vitaxin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; wortmannin; XL518; zanoterone; zeniplatin; zilascorb; zinostatin stimalamer; zinostatin; and zorubicin hydrochloride.

Other exemplary anti-cancer agents useful in the methods described herein include Erbulozole (e.g., R-55104); Dolastatin 10 (e.g., DLS-10 and NSC-376128); Mivobulin isethionate (e.g., CI-980); NSC-639829; Discodermolide (e.g., NVP-XX-A-296); ABT-751 (Abbott; e.g., E-7010); Altorhyrtin A; Altorhyrtin C; Cemadotin hydrochloride (e.g., LU-103793 and NSC-D-669356); CEP 9722; Epothilone A; Epothilone B; Epothilone C; Epothilone D; Epothilone E; Epothilone F; Epothilone B N-oxide; Epothilone A N-oxide; 16-aza-epothilone B; 21-aminoepothilone B; 21-hydroxyepothilone D; 26-fluoroepothilone; Auristatin PE (e.g., NSC-654663); Soblidotin (e.g., TZT-1027); LS-4559-P (Pharmacia; e.g., LS-4577); LS-4578 (Pharmacia; e.g., LS-477-P); LS-4477 (Pharmacia); LS-4559 (Pharmacia); RPR-112378 (Aventis); DZ-3358 (Daiichi); FR-182877 (Fujisawa; e.g., WS-9265B); GS-164 (Takeda); GS-198 (Takeda); KAR-2 (Hungarian Academy of Sciences); BSF-223651 (BASF; e.g., ILX-651 and LU-223651); SAH-49960 (Lilly/Novartis); SDZ-26897(Lilly/Novartis); AM-97 (Armad/Kyowa Hakko); AM-132 (Armad); AM-138 (Armad/Kyowa Hakko); IDN-5005 (Indena); Cryptophycin 52 (e.g., LY-355703); AC-7739 (Ajinomoto; e.g., AVE-8063A and CS-39.HCl); AC-7700 (Ajinomoto; e.g., AVE-8062; AVE-8062A; CS-39-L-Ser.HCl; and RPR-258062A); Vitilevuamide; Tubulysin A; Canadensol; CA-170 (Curis, Inc.); Centaureidin (e.g., NSC-106969); T-138067 (Tularik; e.g., T-67; TL-138067 and TI-138067); COBRA-1 (Parker Hughes Institute; e.g., DDE-261 and WHI-261); H10 (Kansas State University); H16 (Kansas State University); Oncocidin A1 (e.g., BTO-956 and DIME); DDE-313 (Parker Hughes Institute); Fijianolide B; Laulimalide; SPA-2 (Parker Hughes Institute); SPA-1 (Parker Hughes Institute; e.g., SPIKET-P); 3-IAABU (Cytoskeleton/Mt. Sinai School of Medicine; e.g., MF-569); Narcosine (e.g., NSC-5366); Nascapine; D-24851 (Asta Medica); A-105972 (Abbott); Hemiasterlin; 3-BAABU (Cytoskeleton/Mt. Sinai School of Medicine; e.g., MF-191); TMPN (Arizona State University); Vanadocene acetylacetonate; T-138026 (Tularik); Monsatrol; Inanocine (e.g., NSC-698666); 3-IAABE (Cytoskeleton/Mt. Sinai School of Medicine); A-204197 (Abbott); T-607 (Tuiarik; e.g., T-900607); RPR-115781 (Aventis); Eleutherobins (e.g., Desmethyleleutherobin; Desaetyleleutherobin; lsoeleutherobin A; and Z-Eleutherobin); Caribaeoside; Caribaeolin; Halichondrin B; D-64131 (Asta Medica); D-68144 (Asta Medica); Diazonamide A; A-293620 (Abbott); NPI-2350 (Nereus); Taccalonolide A; TUB-245 (Aventis); A-259754 (Abbott); Diozostatin; (−)-Phenylahistin (e.g., NSCL-96F037); D-62638 (Asta Medica); D-62636 (Asta Medica); Myoseverin B; D-43411 (Zentaris; e.g., D-81862); A-289099 (Abbott); A-318315 (Abbott); HTI-286 (e.g., SPA-110; trifluoroacetate salt) (Wyeth); D-82317 (Zentaris); D-82318 (Zentaris); SC-12983 (NCI); Resverastatin phosphate sodium; BPR-OY-007 (National Health Research Institutes); and SSR-250411 (Sanofi)); goserelin; leuprolide; triptolide; homoharringtonine; topotecan; itraconazole; deoxyadenosine; sertraline; pitavastatin; clofazimine; 5-nonyloxytryptamine; vemurafenib; dabrafenib; gefitinib (IRESSA); erlotinib (TARCEVA); cetuximab (ERRITUX); lapatinib (TYKERB); panitumumab (VECTIBIX); vandetanib (CAPRELSA); afatinib/BIBW2992; CI-1033/canertinib; neratinib/HKI-272; CP-724714; TAK-285; AST-1306; ARRY334543; ARRY-380; AG-1478; dacomitinib/PF299804; OSI-420desmethyl erlotinib; AZD8931; AEE726; pelitinib/EKB-569; CUDC-101; WZ8040; WZ4002; WZ3146; AG-490; XL647; PD153035; 5-azathioprine; 5-aza-2′-deoxycytidine; 17-N-Allylamino-17-Demethoxygeldanamycin (17-AAG); 20-epi-1,25 dihydroxyvitamin D3; 5 ethynyluracil; and BMS-599626.

The anti-cancer agent can be a checkpoint inhibitor. The term “checkpoint inhibitor” refers to molecules that totally or partially reduce, inhibit, interfere with or modulate one or more checkpoint proteins. Without being limited by a particular theory, checkpoint proteins regulate T-cell activation or function. Exemplary checkpoint include CTLA-4 and its ligands CD80 and CD86; PD-1 and its ligands PD-L1 and PD-L2; TIM-3; OX40 (CD134); GITR; CD137; CD40; recombinant human interleukin-15 (rhIL-15); and IDO. These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors can include antibodies or are derived from antibodies.

The checkpoint inhibitor can be a CTLA-4 inhibitor. The CTLA-4 inhibitor can be an anti-CTLA-4 antibody. Examples of anti-CTLA-4 antibodies include, but are not limited to, those described in U.S. Pat. Nos: 5,811,097; 5,811,097; 5,855,887; 6,051,227; 6,207,157; 6,682,736; 6,984,720; and 7,605,238, all of which are incorporated herein in their entireties and for all purposes. Exemplary anti-CTLA-4 antibodies include tremelimumab and ipilimumab. Thus, provided herein are methods of treating a cancer by administering a CD44-modulating polypeptide described herein in combination with tremelimumab. In another aspect provided herein are methods of treating cancer described herein by administering a CD44-modulating polypeptide described herein in combination with ipilimumab. The CD44-modulating polypeptide described herein can be a polypeptide of SEQ ID NO:1 or SEQ ID NO:2.

The checkpoint inhibitor can be a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties and for all purposes.

The checkpoint inhibitor can be a PD-1 inhibitor. The checkpoint inhibitor can be an anti-PD-1 antibody. Exemplary PD-1/PD-L1/PD-L2 inhibitors and antibodies include nivolumab, pembrolizumab, AMP-224, pidilizumab, REGN2810, PDR 001, MEDI0680, durvalumab, avelumab, atezolizumab, BMS-936559, or rHIgM12B7A. In one aspect provided herein are methods of treating cancer described herein by administering a CD44-modulating polypeptide described herein in combination with nivolumab. In another aspect provided herein are methods of treating cancer described herein by administering a CD44-modulating polypeptide described herein in combination with pembrolizumab. In another aspect provided herein are methods of treating cancer described herein by administering a CD44-modulating polypeptide described herein in combination with AMP-224. In another aspect provided herein are methods of treating cancer described herein by administering a CD44-modulating polypeptide described herein in combination with pidilizumab. In another aspect provided herein arc methods of treating cancer described herein by administering a CD44-modulating polypeptide described herein in combination with REGN2810. In another aspect provided herein are methods of treating cancer described herein by administering a CD44-modulating polypeptide described herein in combination with PDR, 001. In another aspect provided herein are methods of treating cancer described herein by administering a CD44-modulating polypeptide described herein in combination with MEDI0680. In another aspect provided herein are methods of treating cancer described herein by CD44-modulating polypeptide described herein in combination with durvalumab. In another aspect provided herein are methods of treating cancer described herein by CD44-modulating polypeptide described herein in combination with avelumab. In another aspect provided herein are methods of treating cancer described herein by administering a CD44-modulating polypeptide described herein 1 in combination with atezolizumab. In another aspect provided herein are methods of treating cancer described herein by administering a CD44-modulating polypeptide described herein in combination with BMS-936559. In another aspect provided herein are methods of treating cancer described herein by administering a CD44-modulating polypeptide described herein in combination with rHIgM12B7A. The CD44-modulating polypeptide can be a polypeptide of SEQ ID NO:1 or SEQ ID NO:2. Such combinations can further include administration of one or more radiation therapies described herein.

The checkpoint inhibitor can be a lymphocyte activation gene-3 (LAG-3) inhibitor. Exemplary LAG-3 inhibitors include IMP321 and BMS-986016. Thus, provided herein are methods of treating cancer described herein by administering a CD44-modulating polypeptide described herein in combination with IMP321. Also provided herein are methods of treating cancer described herein by administering a CD44-modulating polypeptide described herein in combination with BMS-986016. The CD44-modulating polypeptide can be a polypeptide of SEQ ID NO:1 or SEQ ID NO:2. Such combinations can further include administration of one or more radiation therapies described herein.

The checkpoint inhibitor can be a B7 inhibitor. In one embodiment, the B7 inhibitor can be a B7-H3 inhibitor or a B7-H4 inhibitor. The B7-H3 inhibitor can be MGA271. MGA271 can be administered in combination with a CD44-modulating polypeptide described herein to treat a cancer described herein in a patient described herein. The CD44-modulating polypeptide can be a polypeptide of SEQ ID NO:1 or SEQ ID NO:2. Such combinations can further include administration of one or more radiation therapies described herein,

In another aspect, the checkpoint inhibitor can be a TIM-3 (T-cell immunoglobulin domain and mucin domain 3) inhibitor (Fourcade et al., J. Exp. Med, 2010, 207, 2175-86; Sakuishi et al., J. Exp. Med, 2010, 207, 2187-94). Provided herein are methods of treating cancer described herein by administering a CD44-modulating polypeptide described herein in combination with TIM-3 inhibitor. The CD44-modulating polypeptide can be a polypeptide of SEQ ID NO:1 or SEQ ID NO:2. Such combinations can further include administration of one or more radiation therapies described herein.

The checkpoint inhibitor can be an OX40 (CD134) agonist. In one embodiment, the checkpoint inhibitor can be an anti-OX40 antibody, such as for example, MEDI6469. Provided herein are methods of treating cancer described herein by administering a CD44-modulating polypeptide described herein in combination with OX40 agonist. Provided herein are methods of treating cancer described herein by administering a CD44-modulating polypeptide described herein in combination with MEDI6469. The CD44-modulating polypeptide can be a polypeptide of SEQ ID NO:1 or SEQ ID NO:2. Such combinations can further include administration of one or more radiation therapies described herein.

In still another aspect, the checkpoint inhibitor can be a GITR agonist. In one embodiment, the checkpoint inhibitor can be an anti-GITR antibody. In one embodiment, the anti-GITR antibody can be TRX518. Provided herein are methods of treating cancer described herein by administering a CD44-modulating polypeptide described herein in combination with anti-GITR antibody. Also provided herein are methods of treating cancer described herein by administering a CD44-modulating polypeptide described herein in combination with TRX518. The CD44-modulating polypeptide can be a polypeptide of SEQ ID NO:1 or SEQ ID NO:2. Such combinations can further include administration of one or more radiation therapies described herein.

In yet another aspect, the checkpoint inhibitor can be a CD137 agonist. In one embodiment, the checkpoint inhibitor can be an anti-CD137 antibody. Exemplary anti-CD137 antibodies include urelumab and PF-05082566. Provided herein are methods of treating cancer described herein by administering a CD44-modulating polypeptide described herein in combination with urelumab. Also provided herein are methods of treating cancer described herein by administering a CD44-modulating polypeptide described herein in combination with PF-05082566. The CD44-modulating polypeptide can be a polypeptide of SEQ ID NO:1 or SEQ ID NO:2. Such combinations can further include administration of one or more radiation therapies described herein.

The checkpoint inhibitor can be a CD40 agonist. In one embodiment, the checkpoint inhibitor can be an anti-CD40 antibody such as, for example, CF-870,893. Provided herein are methods of treating cancer described herein by administering a CD44-modulating polypeptide described herein in combination with CF-870,893. The CD44-modulating polypeptide can be a polypeptide of SEQ ID NO:1 or SEQ ID NO:2. Such combinations can further include administration of one or more radiation therapies described herein.

in yet another aspect, the checkpoint inhibitor can be recombinant human interleukin-15 (rhIL-15). Provided herein are methods of treating cancer described herein by administering a CD44-modulating polypeptide described herein in combination with rhIL-15. The CD44-modulating polypeptide can be a polypeptide of SEQ ID NO:1 or SEQ ID NO:2. Such combinations can further include administration of one or more radiation therapies described herein.

In another aspect, the checkpoint inhibitor can be an IDO inhibitor. Exemplary IDO inhibitors include INCB024360 and indoximod. Provided herein are methods of treating cancer described herein by administering a CD44-modulating polypeptide described herein in combination with INCB024360. Also provided herein are methods of treating cancer described herein by administering a CD44-modulating polypeptide described herein in combination with indoximod. The CD44-modulating polypeptide can be a polypeptide of SEQ ID NO:1 or SEQ ID NO:2. Such combinations can further include administration of one or more radiation therapies described herein.

In certain embodiments the methods described herein include administering two or more anti-cancer agents described above in combination with a CD44-modulating polypeptide described herein. The CD44-modulating polypeptide can be a polypeptide of SEQ ID NO:1. In one example, the CD44-modulating polypeptide can be a polypeptide of SEQ ID NO:1 where the polypeptide contains a substitution of one or more amino acids as described above. The CD44-modulating polypeptide can be a polypeptide of SEQ ID NO:2. Such combinations can include administration of anti-cancer agents in the same class or different classes of agents. Such combinations can further include administration of one or more radiation therapies described herein.

In certain instances, the CD44-modulating polypeptide described herein can be a peptidomimetic as described herein of one or more amino acids sequences set forth herein. For example, a CD44-modulating polypeptide described herein can include N-terminal or C-terminal modifications such as acylation, esterification, carbamation, benzylation, or amidation. In particular embodiments a CD44-modulating polypeptide includes an N-terminal modification. For example, the N-terminal of a CD44-modulating polypeptide described herein can be acylated or modified to a carbamate. In other instances the C-Terminal of a CD44-modulating polypeptide described herein can include modification. Such modification can include amidation. In another example, both the N-terminal and C-terminal of a CD44-modulating polypeptide described herein are modified. In such examples, the N-terminal can be acylated.

In another example, a CD44-modulating polypeptide described herein can include incorporation of one or more D-amino acids. For example, incorporation of D-Lys, D-Ser, or D-Pro can be performed at positions 1, 2, 3, 4, 5, or 6 of SEQ ID NO:1. In one embodiment each amino acid of SEQ ID NO:1 can be replaced with a D-amino acid. Incorporation of a D-amino acid into the sequence can decrease polypeptide degradation and/or enhance the half-life of a therapeutic composition including a CD44-modulating polypeptide described herein. It should be noted that modification of CD44-modulating peptides to include D-amino acids includes only those variants having activity comparable (e.g., about equal to or greater than) that of SEQ ID NO:1.

In another example a CD44-modulating polypeptide includes an amide-bond isostere. An amide-bond isostere refers to an amide backbone replacement such as an carba (ψ[CH₂CH₂]), alkenes (ψ[CH═CH]), alkynes (ψ[C≡CC]), methyleneoxy-(ψ[CH₂O]), methyleneamino-(ψ[CH₂NH]), urea (ψ[CH₂CH₂]), hydrazide (ψ[CONCH₂NCH₂]), ester (ψ[C(O)OCH₂], thioamide (ψ[CSNH]), hydroxyamide (ψ[CON(OH)]) or phosphono (ψ[PO(OH)ONH]).

In certain embodiments, the patient to be treated with a combination therapy comprising a CD44-modulating polypeptide described herein and an anti-cancer agent described herein has not been treated with anti-cancer therapy, radiation therapy, or combination thereof prior to the administration the combination therapy. In certain embodiments, the patient to be treated with a combination therapy comprising a CD44-modulating polypeptide described herein and radiation therapy described herein has not been treated with anti-cancer therapy, radiation therapy, or a combination thereof prior to the administration the combination therapy. A cancer patient can be treatment naive,

In certain embodiments, the patient to be treated with a combination therapy comprising a CD44-modulating polypeptide described herein and an anti-cancer agent described herein has been treated with anti-cancer therapy, radiation therapy, or combination thereof prior to administration of a CD44-modulating polypeptide described herein in combination with an anti-cancer agent described herein. In one example, a patient described herein has been treated with one, two, three, four, five, or more anti-cancer agents. In another example, a patient described herein has been treated with a combination of anti-cancer agents prior to administration of a CD44-modulating polypeptide described herein. In another example, a patient has been treated with radiation therapy (where the patient has optionally been treated as provided above with one or more anti-cancer agents).

A CD44-modulating polypeptide can be a first-line therapy. In cases where the patient has received anti-cancer treatment, radiation therapy, or a combination thereof prior to administration with a CD44-modulating polypeptide, the CD44-modulating polypeptide can be a second, third, fourth or more line treatment. In one example, the CD44-modulating peptide can be SEQ ID NO:1 and can be administered as part of a combination with a first administration of an anti-cancer agent described herein or with a first administration of radiation therapy described herein. In another example, the CD44-modulating polypeptide can be SEQ ID NO:1 and can be administered as part of a combination with one or more anti-cancer agents described herein, radiation therapy described herein, or a combination thereof as a second or third-line or more line therapy. In another example, the CD44-modulating polypeptide can be SEQ ID NO:1 or SEQ ID NO:2 and can be administered in combination with an anti-cancer agent described herein, radiation therapy described herein, or a combination thereof as a last-line therapy.

A CD44-modulating polypeptide described herein can be administered in an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 900 mg, 1000 mg, 1100 mg, 1200 mg, 1300 mg, 1400 mg, or 1500 mg. A CD44-modulating polypeptide described herein can be administered in an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, 200 mg, 250 mg, 300 mg, 350 mg, 400 mg, or 450 mg. A CD44-modulating polypeptide described herein can be administered in an amount of about: 100 mg, 125 mg, 150 mg, 175 mg, 200 mg, 250 mg, 300 mg, 350 mg, or 400 mg.

A CD44-modulating polypeptide described herein can be administered in an amount of at least about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 900 mg, 1000 mg, 1100 mg, 1200 mg, 1300 mg, 1400 mg, or 1500 mg. A CD44-modulating polypeptide described herein can be administered in an amount of at least about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, 200 mg, 250 mg, 300 mg, 350 mg, 400 mg, or 450 mg. A CD44-modulating polypeptide described herein can be administered in an amount of at least about: 100 mg, 125 mg, 150 mg, 175 mg, 200 mg, 250 mg, 300 mg, 350 mg, or 400 mg.

A CD44-modulating polypeptide can be administered in an amount from about: 1 to about 500 mg: 50 mg to about 500 mg; 100 mg to about 500 mg; 150 mg to about 500 mg; 250 mg to about 500 mg; or about 300 mg to about 500 mg. A CD44-modulating polypeptide can be administered in an amount from about: 100 mg to about 200 mg; 100 mg to about 250 mg; 100 mg to about 300 mg; or 100 mg to about 400 mg.

A CD44-modulating polypeptide can be administered in an amount from about: 1 mg to about 1500 mg: 50 mg to about 1500 mg; 100 mg to about 1500 mg; 250 mg to about 1500 mg; 500 mg to about 1500 mg; or about 1000 mg to about 1500 mg. A CD44-modulating polypeptide can be administered in an amount from about: 100 mg to about 2000 mg; 100 mg to about 3000 mg; 100 mg to about 4000 mg; 100 mg to about 5000 mg; 100 mg to about 6000 mg; 100 mg to about 7000 mg; 100 mg to about 8000 mg; 100 mg to about 9000 mg; or 100 mg to about 10,000 mg. A CD44-modulating polypeptide can be administered in an amount from about: 500 mg to about 2000 mg; 500 mg to about 1750 mg; 1000 mg to about 2000 mg; 1000 mg to about 1500 mg; 1200 mg to about 1800 mg; or about 1300 mg to about 1500 mg. A CD44-modulating polypeptide can be administered in an amount from about: 1000 mg to 10000 mg; 2000 mg to 10000 mg; 1000 mg to 7500 mg; 1000 mg to 5000 mg; 750 mg to about 4000 mg; or 2000 mg to 5000 mg.

A CD44-modulating polypeptide described herein can be administered as described herein in an amount of about: 25 mg/day to about 1500 mg/day; 25 mg/day to about 1200 mg/day; 25 mg/day to about 1000 mg/day; 25 mg/day to about 750 mg/day; 25 mg/day to about 500 mg/day; 25 mg/day to about 300 mg/day; 25 mg/day to about 250 mg/day; 25 mg/day to about 150 mg/day; 50 mg/day to about 1000 mg/day; 50 mg/day to about 750 mg/day; 50 mg/day to about 500 mg/day; 50 mg/day to about 300 mg/day; 50 mg/day to about 250 mg/day; 50 mg/day to about 150 mg/day; 100 mg/day to about 500 mg/day; 100 mg/day to about 300 mg/day; or about 150 mg/day to about 300 mg/day.

The polypeptide of SEQ ID NO:1 or SEQ ID NO:2 can be administered in an amount of about: 50 mg, 100 mg, 150 mg, 200 mg, 250 mg, or 300 mg. The polypeptide of SEQ ID NO:1 or SEQ ID NO:2 can be administered as described herein in an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 900 mg, 1000 mg, 1100 mg, 1200 mg, 1300 mg, 1400 mg, or 1500 mg. The polypeptide of SEQ ID NO:1 or SEQ ID NO:2 can be administered in an amount of about: 50 mg, 100 mg, 150 mg, 200 mg, 250 mg, or 300 mg. The polypeptide of SEQ ID NO:1 or SEQ ID NO:2 can be administered in an amount of about: 100 mg, 200 mg, 300 mg, 400 mg, or 500 mg.

The polypeptide of SEQ ID NO:1 or SEQ ID NO:2 can be administered in an amount of at least about: 50 mg, 100 mg, 150 mg, 200 mg, 250 mg, or 300 mg. The polypeptide of SEQ ID NO:1 or SEQ ID NO:2 can be administered as described herein in an amount of at least about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 900 mg, 1000 mg, 1100 mg, 1200 mg, 1300 mg, 1400 mg, or 1500 mg. The polypeptide of SEQ ID NO:1 or SEQ ID NO:2 can be administered in an amount of at least about: 50 mg, 100 mg, 150 mg, 200 mg, 250 mg, or 300 mg. The polypeptide of SEQ ID NO:1 or SEQ ID NO:2 can be administered in an amount of at least about: 100 mg, 200 mg, 300 mg, 400 mg, or 500 mg.

The polypeptide of SEQ ID NO:1 or SEQ ID NO:2 can be administered as described herein in an amount of about: 25 mg/day to about 1500 mg/day; 25 mg/day to about 1200 mg/day; 25 mg/day to about 1000 mg/day; 25 mg/day to about 750 mg/day; 25 mg/day to about 500 mg/day; 25 mg/day to about 300 mg/day; 25 mg/day to about 250 mg/day; 25 mg/day to about 150 mg/day; 50 mg/day to about 1000 mg/day; 50 mg/day to about 750 mg/day; 50 mg/day to about 500 mg/day; 50 mg/day to about 300 mg/day; 50 mg/day to about 250 mg/day; 50 mg/day to about 150 mg/day; 100 mg/day to about 500 mg/day; 100 mg/day to about 300 mg/day; or about 150 mg/day to about 300 mg/day.

In certain instances it can be useful to administer the CD44-modulating polypeptide as an amount relative to the weight of the patient. A polypeptide of SEQ ID NO:1 or SEQ ID NO:2 can be administered as an amount relative to the weight of the patient. A polypeptide of SEQ ID NO:1 or SEQ ID NO:2 can be administered in an amount of about: 0.5 mg/kg, 1 mg/kg, 2 mg/kg, 3 mg/kg, 5 mg/kg, 6 mg/kg, 7 mg/kg, 8 mg/kg, 9 mg/kg, 10 mg/kg, 15 mg/kg, or 20 mg/kg. In certain instances the polypeptide of SEQ ID NO:1 or SEQ ID NO:2 can be administered in an amount of about 0.5 mg/kg to about 20 mg/kg; 0.5 mg/kg to about 10 mg/kg, 0.5 mg/kg to about 7.5 mg/kg; 0.5 mg/kg to about 5 mg/kg; or about 0.5 mg/kg to about 1 mg/kg.

CD44-modulating polypeptides can be administered in a treatment regimen that includes administration of the polypeptide in any number of days, weeks, or months and over any period of time, typically until disease lapse, unacceptable toxicity, patient intolerance, or onset of disease symptoms (e.g. relapse or loss of efficacy). CD44-modulating polypeptides described herein and useful in the methods of treating cancers described herein can be administered at any frequency as described herein such as, for example, once a day (QD), every other day (Q2D), twice daily (BID), once a week (QW), twice weekly (BIW), three times a week (TIW, every other week (Q2W), every three weeks (Q3W), or monthly (QM). In one example a CD44-modulating polypeptide described herein can be administered QD for at least 5, 10, 15, 21, 28, 30, 31, 45, 60, 90, 120, 180, or 200 days. In certain instances the CD44-modulating polypeptide can be administered QD for at least 30 or 60 days. In another instance the CD44-modulating polypeptide can be administered QD for at least 90 or 180 days. In one example the CD44-modulating polypeptide can be administered QD until progression of disease or toxicity development.

In another example, a CD44-modulating polypeptide can be administered as maintenance therapy before, during, or after treatment with an anti-cancer agent described herein, radiation therapy described herein, or a combination thereof. Maintenance therapy refers to long term (e.g., 6 months, or 1, 2, 3, 4, 5, 6 or more years) treatment following a treatment regimen for cancer, such as those described herein, that is intended to keep the cancer in remission. Maintenance therapy can be administered indefinitely following a treatment regimen described herein. In certain instances, CD44-modulating polypeptides have little or no toxicity to the patient and continual administration, even after ending a treatment with an anti-cancer agent described herein, radiation therapy described herein, or a combination thereof. In one example, a CD44-modulating polypeptide can be administered as a maintenance therapy after the course of a regimen described herein is completed.

In one embodiment, a CD44-modulating polypeptide can be administered QD at an amount of about 100 mg to 400 mg. In one embodiment, a CD44-modulating polypeptide can be administered QD at an amount of about 1000 mg to 2000 mg. In one embodiment, a CD44-modulating polypeptide can be administered QD at an amount of about 150 mg to 300 mg. In one embodiment, a CD44-modulating polypeptide can be administered QD at an amount of about 100 mg to about 200 mg or about 250 mg to about 350 mg.

In one example, a CD44-modulating polypeptide can be administered BID at an amount of about 100 mg to 400 mg. In one embodiment, a CD44-modulating polypeptide can be administered BID at an amount of about 1000 mg to 2000 mg. In one embodiment, a CD44-modulating polypeptide can be administered BID at an amount of about 150 mg to 300 mg. In one embodiment, a CD44-modulating polypeptide can be administered BID at an amount of about 100 mg to about 200 mg or about 250 mg to about 350 mg. In one embodiment a CD44-modulating polypeptide can be administered BID at an amount of about 150 mg.

In another example, a CD44-modulating polypeptide can be administered multiple times a day such that the total amount administered in one day (e.g., about 24 hours) can be about 100 mg to about 400 mg. A CD44-modulating polypeptide can be administered multiple times such that the total amount administered in one day can be about 100 mg, 150 mg, 200 mg, 250 mg, 300 mg, or about 350 mg.

In another example, a CD44-modulating polypeptide described herein can be administered Q2D. The duration of administration can be the same as described above for QD administration. For example, Q2D administration can be performed for at least 30, 60, 90, 120, or 180 days. In one embodiment, the amount of CD44-modulating polypeptide administered Q2D can be equivalent to an amount administered QD. In another embodiment, the amount of CD44-modulating polypeptide administered Q2D can be greater than an amount administered QD.

In another example, a CD44-modulating polypeptide can be administered once weekly QW)). Once weekly administration can be performed for at least 1, 2, 3, 4, 5, 6, 7, 8 9, 10, 11, 12, 16, 20, or 24 weeks.

QD or BID administration of CD44-modulating polypeptides can be performed in a cyclic regimen that includes administration as described above, e.g. 14, 21, or 28 days, and the cycle repeated continually. In one example, QD or BID administration of a CD44-modulating polypeptide can be continual without any rest or off period of administration. In another example, administration includes a rest period or off period can be included between each cycle. A rest or off period can be about 1 to 7 days.

Anti-cancer agents described herein and useful in the methods of treating cancers described herein can be administered in any amount. In certain instances the effective amount of an anti-cancer agent described herein can be determined as an amount provided in a package insert provided with the agent.

Anti-cancer agents described herein and useful in the methods of treating cancers described herein can be administered at any frequency as described herein such as, for example, once a day (QD), twice daily (BID), once a week (QW), twice weekly (BIW), three times a week (TIW), every other week (Q2W), every three weeks (Q3W), or monthly (QM). For example, the anti-cancer agent can be administered BID. An anti-cancer agent can be administered TIW. In certain instances, the anti-cancer agent can be administered 2 to 3 times a week. An anti-cancer agent can be administered QD. An anti-cancer agent can be administered QD for about: 1 day to about 7 days, 1 day to about 14 days, 1 day to about 21 days, 1 day to about 28 days, or daily until disease progression or unacceptable toxicity. Such administration can be performed in cycles such that a 14, 12, or 28 day cycle of treatment can be repeated. When administration is cyclic in nature, a rest period of 1 to 7 days can be included between cycles. The administration of an anti-cancer agent described herein can, in part, depend upon the tolerance of the patient where greater tolerance can allow greater or more frequent administration. Alternatively, where a patient shows poor tolerance to an anti-cancer agent described herein, a less amount of the agent or a less frequent dosing can be performed. An anti-cancer agent can be administered in any regimen as described herein.

For example, an anti-cancer agent described herein can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QD. In one example, an anti-cancer agent described herein includes an agent present at an amount of about: 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 8000 mg; 9000 mg, or 10000 mg, QD. For example, an anti-cancer agent described herein can be administered at an amount of about: 1 mg, 2 mg, 3 mg, 4 mg, 5 mg, 10 mg, 15 mg, 20 mg, 25 mg, 30 mg; 35 mg; 40 mg; 45 mg, 50 mg, 60 mg, 70 mg, 80 mg, 85 mg, 90 mg, 100 mg, 125 mg, 150 mg, 175 mg, or 200 mg, BIW. In one example, an anti-cancer agent described herein includes an agent present at an amount of about: 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 8000 mg, 9000 mg, or 10000 mg, BIW. For example, an anti-cancer agent described herein can be administered at an amount of about: 1 mg, 2 mg, 3 mg, 4 mg, 5 mg, 10 mg, 15 mg, 20 mg, 25 mg, 30 mg, 35 mg; 40 mg; 45 mg; 50 mg, 60 mg, 70 mg, 80 mg, 85 mg, 90 mg, 100 mg, 125 mg, 150 mg, 175 mg, or 200 mg, TIW. In one example, an anti-cancer agent described herein includes an agent present at an amount of about: 100 mg; 200 mg; 300 mg; 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 8000 mg, 9000 mg, or 10000 mg, TIW. For example, an anti-cancer agent described herein can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QW. In one example, an anti-cancer agent described herein includes an agent present at an amount of about: 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 8000 mg, 9000 mg, or 10000 mg, QW. For example, an anti-cancer agent described herein can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, Q2W. In one example, an anti-cancer agent described herein includes an agent present at an amount of about: 100 mg, 200 mg, 300 mg, 400 mg, 500 mg, 600 mg, 700 mg, 800 mg, 900 mg; 1000 mg; 1500 mg, 2000 mg, 2500 mg, 3000 mg, 3500 mg, 4000 mg, 4500 mg, 5000 mg, 5500 mg, 6000 mg, 6500 mg, 7000 mg, 8000 mg, 9000 mg, or 10000 mg, Q2W. Administration of an anti-cancer agent described herein can be continuous. Administration of an anti-cancer agent described herein can be intermittent.

For example, an anti-cancer agent described herein can be administered at an amount of about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QD. For example, an anti-cancer agent described herein can be administered at an amount of about: 0.0001 mg/kg about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, BIW. For example, an anti-cancer agent described herein can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, TIW. For example, an anti-cancer agent described herein can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QW. For example, an anti-cancer agent described herein can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, Q2W. Administration of an anti-cancer agent described herein can be continuous. Administration of an anti-cancer agent described herein can be intermittent.

For example, an anti-cancer agent described herein can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QD. For example, an anti-cancer agent described herein can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, BIW. For example, an anti-cancer agent described herein can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, TIW. For example, an anti-cancer agent described herein can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QW. For example, an anti-cancer agent described herein can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, Q2W.

An anti-cancer agent described herein can be administered as an intravenous infusion over about 10, 20, 30, 40, 50, or 60 or more minutes. An anti-cancer agent described herein can be administered as an intravenous infusion over about 60 minutes according to a regimen and time period set forth above. An anti-cancer agent described herein can be administered as an intravenous infusion according to a package insert. An anti-cancer agent described herein can be administered as an oral dosage form. Such forms include tablets, capsules, and sachets as described herein and understood in the art. When administered as an oral dosage form, the oral dosage form can be administered according to a regimen or time period as described herein. In certain instances, the anti-cancer agent can be administered as an oral dosage form according to a package insert.

Checkpoint inhibitors described herein for use in the methods described herein can be administered in amounts from about 0.005 to about 2,000 mg per day, from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, or from about 0.05 to about 10 mg per day. In one embodiment a checkpoint inhibitor described herein can be administered in an amount from about 500 mg to about 2500 mg, 750 mg to about 2250 mg, 1000 mg to about 2000 mg, or about 1200 mg to about 1800 mg.

Checkpoint inhibitors described herein can be administered in a therapeutically effective amount of about 100, 200, 300, 400, 500, 600, 700, 800, 900, 1000, 1100, 1200, 1300, 1400, 1500, 1600, 1700, 1800, 1900, 2000, 2100, 2200, 2300, 2400, or about 2500 mg. In certain embodiments the checkpoint inhibitor can be administered in a therapeutically effective amount of about 1000, 1250, 1500, 1750, or 2000 mg.

Dosages of anti-cancer agents described herein can be modified (e.g., increased or decreased dosage) during treatment as set, forth herein and understood in the art.

An anti-cancer agent for use in combination therapies described herein can independently be administered once daily (QD), or divided into multiple daily doses such as twice daily (BID), three times daily (TM), and four times daily as part of a combination therapy described herein. In addition, the administration can be continuous (i.e., daily for consecutive days or every day), intermittent, e.g., in cycles (i.e., including days, weeks, or months of rest without drug)). As used herein, the term “daily” is intended to mean that an anti-cancer agent is administered once or more than once each day, for example, for a period of time. The term “monthly” is intended to mean that an anti-cancer agent is administered once a month or about every 4 weeks for an uninterrupted period of time equal to the number of cycles of administration. The term “continuous” is intended to mean that an anti-cancer agent is administered daily for an uninterrupted period of at least 10 days to 52 weeks. The term “intermittent” or “intermittently” as used herein is intended to mean stopping and starting at either regular or irregular intervals. For example, intermittent administration of an anti-cancer agent for use in methods described herein can be administered for one to six days per week, administration in cycles (e.g., daily administration for two to eight consecutive weeks, then a rest period with no administration for up to one week), or administration on alternate days. For example, intermittent administration of an anti-cancer agent for use in methods described herein can be administered for once per month, administration in cycles (e.g., monthly administration for two to twelve cycles).

In some embodiments, the frequency of administration of an anti-cancer agent can be in the range of about a daily dose to about a monthly dose. In certain embodiments, administration of an anti-cancer agent can be once a day, twice a day, three times a day, four times a day, once every other day, twice a week, once every week, once every two weeks, once every three weeks, or once every four weeks. In one embodiment, a polypeptide of SEQ ID NO:1 or SEQ ID NO:2 can be administered in combination with an anti-cancer agent that can be administered once a month. In another embodiment, a polypeptide of SEQ ID NO:1 or SEQ ID NO:2 can be administered in combination with an anti-cancer agent that can be administered twice a month. In yet another embodiment, a polypeptide of SEQ ID NO:1 or SEQ ID NO:2 can be administered in combination with an anti-cancer agent that can be administered three times a month. In still another embodiment, a polypeptide of SEQ ID NO:1 or SEQ ID NO:2 can be administered in combination with an anti-cancer agent that can be administered four times a month (e.g., weekly). In another embodiment, a polypeptide of SEQ ID NO:1 or SEQ ID NO:2 can be administered in combination with an anti-cancer agent that can be administered two, three, four, five, or six times a week. In still another embodiment, a polypeptide of SEQ ID NO:1 or SEQ ID NO:2 can be administered in combination with an anti-cancer agent that can be administered daily.

When the anti-cancer agent can be a checkpoint inhibitor, the checkpoint inhibitor can be an antibody present in an amount as a measure with regards to the weight of the patient in need thereof. For example, the antibody can be present in an amount of about: 0.1 mg/kg to about 30 mg/kg, 0.1 mg/kg to about 25 mg/kg, 0.1 mg/kg to about 20 mg/kg, 0.1 mg/kg to about 15 mg/kg, 0.1 mg/kg to about 10 mg/kg, 0.1 mg/kg to about 7.5 mg/kg, 0.1 mg/kg to about 5 mg/kg, 0.1 mg/kg to about 2.5 mg/kg, or about 0.1 mg/kg to about 1 mg/kg. The antibody can be present in an amount of about: 0.5 mg/kg to about 30 mg/kg, 0.5 mg/kg to about 25 mg/kg, 0.5 mg/kg to about 20 mg/kg, 0.5 mg/kg to about 15 mg/kg, 0.5 mg/kg to about 10 mg/kg, 0.5 mg/kg to about 7.5 mg/kg, 0.5 mg/kg to about 5 mg/kg, 0.5 mg/kg to about 2.5 mg/kg, or about 0.5 mg/kg to about 1 mg/kg. The antibody can be present in an amount of about 0.5 mg/kg to about 5 mg/kg or about 0.1 mg/kg to about 10 mg/kg. The antibody can be present in an amount of about 0.5 mg/kg to about 15 mg/kg or about 0.1 mg/kg to about 20 mg/kg.

In still other embodiments, the antibody can be present at an amount of about: 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg or 30 mg/kg. The antibody can be present at an amount of about: 1 mg/kg, 2 mg/kg, 3 mg/kg, or 5 mg/kg.

The checkpoint inhibitor antibody can be present in the combination at an amount of about: 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, 200 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. The antibody can be present in the combination at an amount of about: 1 mg to about 10 mg, 10 mg to about 20 mg, 25 mg to about 50 mg, 30 mg to about 60 mg, 40 mg to about 50 mg, 50 mg to about 100 mg, 75 mg to about 150 mg, 100 mg to about 200 mg, 200 mg to about 500 mg, 500 mg to about 1000 mg, 1000 mg to about 1200 mg, 1000 mg to about 1500 mg, 1200 mg to about 1500 mg, or 1500 mg to about 2000 mg.

The antibody can be present in the combination in an amount of about: 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 400 mg/mL, or 500 mg/mL. In one embodiment, the antibody can be present in the combination in an amount of about: 1 mg/mL to about 10 mg/mL, 5 mg/mL to about 10 mg/mL, 5 mg/mL: to about 15 mg/mL, 10 mg/mL to about 25 mg/mL; 20 mg/mL: to about 30 mg/mL; 25 mg/mL, to about 50 mg/mL, or 50 mg/mL to about 100 mg/mL.

In certain instances the therapeutically effective amount of an antibody can be determined as an amount provided in a package insert provided with the antibody. The term package insert refers to instructions customarily included in commercial packages of medicaments approved by the FDA or a similar regulatory agency of a country other than the USA, which contains information about, for example, the usage, dosage, administration, contraindications, and/or warnings concerning the use of such medicaments.

A radiation therapy for use as described herein can independently be administered once daily (QD), or divided into multiple daily doses such as twice daily (BID), three times daily (TID), and four times daily as part of a combination therapy described herein. In addition, the administration can be continuous (i.e., daily for consecutive days or every day), intermittent, e.g., in cycles (i.e., including days, weeks, or months of rest without therapy). For example, intermittent administration of radiation therapy for use in methods described herein can be administered for one to six days per week, administration in cycles (e.g., daily administration for two to eight consecutive weeks, then a rest period with no administration for up to one week), or administration on alternate days. In another example, radiation therapy can be administered QD for at least 5 consecutive days, where the administration is performed in any number of cycles, such as, 2-12 cycles.

In one embodiment, a polypeptide of SEQ ID NO:1 or SEQ ID NO:2 can be administered in combination with radiation therapy that can be administered once a month. In another embodiment, a polypeptide of SEQ ID NO:1 or SEQ ID NO:2 can be administered in combination with radiation therapy that can be administered twice a month. In yet another embodiment, a polypeptide of SEQ ID NO:1 or SEQ ID NO:2 can be administered in combination with radiation therapy that can be administered three times a month. In still another embodiment, a polypeptide of SEQ ID NO:1 or SEQ ID NO:2 can be administered in combination with radiation therapy that can be administered four times a month (e.g., weekly). In another embodiment, a polypeptide of SEQ ID NO:1 or SEQ ID NO:2 can be administered in combination with radiation therapy that can be administered two, three, four, five, or six times a week. In still another embodiment, a polypeptide of SEQ ID NO:1 or SEQ ID NO:2 can be administered in combination with radiation therapy that can be administered daily.

In some embodiments, the dose of ionizing radiation ranges between about 2 KeV to about 25 000 KeV or more, between about 2 KeV to about 6000 KeV or more (e.g. from a linear accelerator source), or between about 2 KeV to about 1500 KeV (such as from a radioisotopic source, like cobalt 60). In some cases, cumulative external irradiation of a patient comprises a dose of 1 to 100 Gy or more, such as from 1 to 60 Gy or more. In certain embodiments, the dose of radiation therapy is less than 90 Gy, such as less than 80 Gy, 70 Gy, 60 Gy, 50 Gy, 40 Gy, 30 Gy, 20 Gy, or less. In certain embodiments the dose or radiation therapy is between about 10 to 100 Gy, such as from about 20 to 80 Gy, about 30 to 70 Gy, or about 40 to 60 Gy. An external radiation dose may be administered in fractional doses, such as from 1 to 60 fractional doses, or from 5 to 30 fractional doses.

Radiation therapy (e.g. fractional doses) may be administered periodically, such about every 1, 2, 3, 4. 5, 6, 12, 18, or 24 hours, about every 1, 2, 3, 4, 5, 6, 7, 14, 21, 28, or 30 days, about every 1, 2, 3, or 4 weeks, or about every 1, 2, 3, 4, 5, 6, or 12 months. In certain embodiments, fractionized doses are administered with about 1.5 to about 2.5 Gy per fraction (e.g. about 1.5 Gy, 1.6 Gy, 1.7 Gy, 1.8 Gy, 1.9 Gy, 2.0 Gy, 2.1 Gy, 2.2 Gy, 2.3 Gy, 2.4 Gy, or 2.5 Gy per fractionized dose). Fractionated doses of radiation therapy may be administered at intervals. In certain embodiments, the fractionized doses are administered over a period of minutes, hours, or weeks such as 1 to 26 weeks, 1 to 15 weeks, or 2 to 12 weeks. In certain embodiments, the fractionized doses are administered over a period less than about 15 weeks, such as less than about 14, 13, 12, 11, 10, 9, 8, 7, 6, 5, 4, or fewer weeks. In other embodiments, the radiation therapy is administered in a single dosage rather than in fractionized doses. For example, the single dose may be administered with about 1-30 Gy per dose, such as from 5-20 Gy or such as about 10-15 Gy.

The polypeptide of SEQ ID NO:1 or SEQ ID NO:2 can be provided in amounts that are synergistic with the amount of the anti-cancer agent, radiation therapy described herein, or a combination thereof. The term synergistic refers to a combination therapy of a polypeptide of SEQ ID NO:1 or SEQ ID NO:2 and an anti-cancer described herein, radiation therapy described herein, or a combination thereof or including combination of regimens such as those described herein that can be more effective than the additive effects of each individual therapy or regimen. The polypeptide of SEQ ID NO:1 or SEQ ID NO:2 can be synergistic with one or more anti-cancer agents, radiation therapy(ies), or combination there where the administration regimen and dosing regimen of the agents provides a synergistic effect.

The CD44-modulating polypeptides described herein can be useful as imaging agents. In such instances, a CD44-modulating polypeptide described herein can be derivatized or bound to a label for detection. Examples of suitable detectable labels include, but are not limited to, radioactive, fluorogenic, chromogenic, or other chemical labels. Useful radiolabels, which are detected by a gamma counter or a scintillation counter or by autoradiography include isotopic labels such as ³H, ¹²⁵ I, ¹³¹I, ³⁵S and ¹⁴C. In addition, ¹³¹I is also useful as a therapeutic isotope. Common fluorescent labels include fluorescein isothiocyanate, rhodamine, phycoerythrin, phycocyanin, allophycocyanin, o-phthaldehyde and fluorescamine. The CD44-modulating polypeptide can be a polypeptide of SEQ ID NO:1 or SEQ ID NO:2.

Fluorescein, fluorescein derivatives and fluorescein-like molecules such as OREGON GREEN™ and its derivatives, RHODAMINE GREEN™ and RHODL GREEN™, are typically coupled to amine groups using the isocyanate, succinimidyl ester or dichlorotriazinyl-reactive groups. Their spectra are typically not affected by changes in pH between 4 and 10, an important advantage over the fluoresceins for many biological applications: Exemplary fluorophores include tetramethylrhodamines, X-rhodamines and Texas Red derivatives cascade blue, coumarin derivatives, naphthalenes, pyrenes and pyridyloxazole derivatives. Those skilled in the art will recognize that known fluorescent reagents modify groups other than amines, such as thiols, alcohols, aldehydes, ketones, carboxylic acids and amides. Hence, fluorescent substrates can readily be designed and synthesized using these other reactive groups.

CD44-modulating polypeptides described herein can also be labeled for detection using fluorescence-emitting metals such as ¹⁵²Eu, or others of the lanthanide series. These metals can be attached to the peptide using such metal chelating groups as diethylenetriaminepentaacetic acid (DTPA) or ethylenediaminetetraacetic acid (EDTA). CD44-modulating polypeptides described herein can be made detectable by coupling it to a chemiluminescent compound. Exemplary chemiluminescers include luminol, isoluminol, theromatic acridinium ester, imidazole, acridinium salt and oxalate ester. Bioluminescence is a type of chemiluminescence found in biological systems in which a catalytic polypeptide increases the efficiency of the chemiluminescent reaction. The presence of a bioluminescent polypeptide is determined by detecting the presence of luminescence. Exemplary bioluminescent compounds for purposes of labeling are luciferin, luciferase and aequorin. The CD44-modulating polypeptide can be a polypeptide of SEQ ID NO:1 or SEQ ID NO:2.

In yet another embodiment, colorimetric detection is used, based on chromogenic compounds (chromophores) with high extinction coefficients.

Detection of the labeled CD44-modulating polypeptides described herein can be accomplished by removing a histological specimen from a subject and examining it by microscopy under appropriate conditions to detect the label. Those of ordinary skill will readily perceive that any of a wide variety of histological methods (such as staining procedures) can be modified in order to achieve such in situ detection. In another embodiment, detection of the labeled CD44-modulating polypeptide can be performed in vivo using techniques known in the art.

There are many different labels and methods of labeling known to those of ordinary skill in the art. Examples of the types of labels which can be used in the present invention include radioactive isotopes, paramagnetic isotopes, and compounds which can be imaged by positron emission tomography (PET). For example, for diagnostic in vivo radioimaging, the type of detection instrument available is a major factor in selecting a given radionuclide. The radionuclide chosen should have a type of decay which is detectable by a given type of instrument. One other factor in selecting a radionuclide for in vivo diagnosis is that the half-life of a radionuclide be long enough so that it is still detectable at the time of maximum uptake by the target issue, but short enough so that deleterious radiation of the host is minimized.

For in vivo diagnosis, radionuclides can be bound to a CD44-modulating polypeptide described herein either directly or indirectly by using an intermediary functional group. Intermediary functional groups that are often used to bind radioisotopes, which exist as metallic ions, to peptides are the chelating agents, DTPA and EDTA. Examples of metallic ions which can be bound to peptides are ⁹⁹Tc, ¹²³I, ¹¹¹In, ¹³¹I, ⁹⁷Ru, ⁶⁷Cu, ⁶⁷Ga, ¹²⁵I, ⁶⁸Ga, ⁷²As, ⁸⁹Zr, and ²⁰¹ Tl.

Combinations described herein that include a CD44-modulating polypeptide described herein and an anti-cancer described herein can be provided as a pharmaceutical composition suitable for administration via any route to a patient described herein including but not limited to: oral, mucosal (e.g., nasal, inhalation, pulmonary, sublingual, vaginal, buccal, or rectal), parenteral (e.g., subcutaneous, intravenous, bolus injection, intramuscular, or intra-arterial), topical (e.g., eye drops or other ophthalmic preparations), transdermal or transcutaneous administration to a patient. The CD44-modulating polypeptide can be a polypeptide of SEQ ID NO:1 or SEQ ID NO:2. In particular embodiments, a CD44-modulating polypeptide described herein can be formulated as a component of a pharmaceutical composition suitable for transdermal delivery.

Exemplary of dosage forms include: transdermal systems, tablets; caplets; capsules (e.g., gelatin capsules); cachets; lozenges; suppositories; powders; gels; liquid dosage forms suitable for parenteral administration to a patient; and sterile solids (e.g., crystalline or amorphous solids) that can be reconstituted to provide liquid dosage forms suitable for parenteral administration to a patient. In certain embodiments, the CD44-modulating polypeptide described herein can be formulated for parenteral or oral administration. In certain embodiments, the anti-cancer agent described herein can be formulated for transdermal, parenteral, nasal, or oral administration. In certain embodiments, the CD44-modulating polypeptide can be formulated for parenteral or transdermal dosing. In another embodiment, the CD44-modulating polypeptide can be formulated for nasal or parenteral administration.

In one example, the CD44-modulating polypeptide can be formulated for transdermal administration. Transdermal administration includes extended/sustained release transdermal devices (e.g., release of the polypeptide for a period of more than about 1 hour or 1 or more days) and immediate release transdermal devices (e.g., release of the polypeptide over a period of time of less than about 1 hour). In certain instances the CD44-modulating polypeptide can be administered using an immediate release transdermal device that releases the CD44-modulating polypeptide over the course of about 0.5, 1, 2, 3, 4, 5, 6, 7, 8, 9, 10, 11, 12, 13, 14, 15, 20, 25, 30, 45, or 60 minutes. For example, the immediate release transdermal device can release the CD44-modulating polypeptide over a period of time of less than about 60 min; less than about 30 min; less than about 20 min, less than about 15 min; less than about 10 min; less than about 5 min; less than about 3 min; less than about 2 min; less than about 1 mitt; or less than about 0.5 min. In one embodiment, transdermal delivery of a CD44-modulating polypeptide described herein reduces metabolic degradation of the CD44-modulating polypeptide.

In certain instances, transdermal administration can be advantageous for promoting patient compliance because transdermal administration is noninvasive and the administration can be completed in less than a time period set forth above. In such embodiments, the amount of the CD44-modulating polypeptide administered to a patient can be increased by transdermal administration. Patient compliance is often an impetus for failed therapies because fear, pain, and the process of administration can deter patient compliance. Transdermal administration of CD44-modulating polypeptides described herein can increase patient compliance and increase treatment success.

Transdermal administration can include administration of a CD44-modulating polypeptide in a volume of less than about 2 mL (e.g., less than about 2, 1.5, 1, or 0.5 mL) over a period of time provided above. In one example, CD44-modulating polypeptides described herein are administered using a microstructured (e.g., microneedle or hollow point microneedle) transdermal system (3M). Compositions including CD44-modulating polypeptides for transdermal delivery can be formulated as described herein for transdermal delivery. In certain instances, a CD44-modulating polypeptide and an anti-cancer agent can be formulated for transdermal administration.

When CD44-modulating polypeptides described herein are administered transdermally, the transdermal device can provide an extended or sustained release of a CD44-modulating polypeptide. Such transdermal devices can provide transdermal delivery over a time of about 1, 2, 3, 4, 5, 6, 7, or more days. In one example, transdermal delivery occurs over at least 3 to 7 days. In another example, transdermal delivery occurs over at least 15 to 30 days. Systems and devices for transdermal delivery are known and used in the art.

In another embodiment, a CD44-modulating polypeptide can be formulated for intranasal delivery. A CD44-modulating polypeptide can be formulated in a sufficient volume to permit intranasal administration. The volume can be sufficient to allow a plurality of daily administrations to arrive at a total amount of administration of a CD44-modulating polypeptide in an amount described herein. For example, the volume can be sufficient for 1, 2, 3, 4, or more daily administrations intranasal to arrive at an amount of a CD44-modulating polypeptide provided herein.

In one example, the CD44-modulating polypeptide can be formulated for intranasal administration for a total daily dose of about 100 mg to about 400 mg. In another example, the CD44-modulating polypeptide can be formulated for intranasal administration every 2, 4, 6, 8, or 10 hours. Devices for intranasal administration are known in the art. For example, such administration can be performed using atomization and, for example, an atomizer. In one example a CD44-modulating polypeptide can be administered at a total amount of about 300 mg per day by intranasal delivery of an amount of about 75 mg every 6 hours.

The CD44-modulating polypeptide described herein and anti-cancer agents described herein can be formulated the same (e.g., both agents formulated for parenteral administration or oral administration). In certain embodiments, a CD44-modulating polypeptide can be formulated for transdermal delivery and an anti-cancer agent can be formulated for either oral or parenteral administration. The CD44-modulating polypeptide described herein and an anti-cancer agent described herein can be formulated in the same dosage form or as separate dosage forms. The CD44-modulating polypeptide can be a polypeptide of SEQ ID NO:1 or SEQ ID NO:2.

Pharmaceutical compositions and dosage forms described herein typically include one or more excipients. Suitable excipients are well known to those skilled in the art of pharmacy. Whether a particular excipient is suitable for incorporation into a pharmaceutical composition or dosage form depends on a variety of factors such as, for example, the intended route of administration to the patient. Pharmaceutical compositions described herein can include other agents such as stabilizers, lubricants, buffers, and disintegrants that can reduce the rate by which an active ingredient can decompose in a particular formulation. Pharmaceutical compositions described herein can also include other agents such as gelatins, cellulose, thickening/thinning agents, and penetration enhancers (e.g. suifoxides, ethanol, PEG, oleic acid) that can control the rate an active ingredient can be delivered by, for example, a transdermal system.

Pharmaceutical compositions described herein can in certain instances include additional active agents other than those in the combinations described herein (e.g., an anti-cancer agent such as those described herein) in an amount provided herein.

Anti-cancer agents described herein can be provided in forms convenient to or facilitate their administration to a patient, or for example, according to the formulation provided with a package insert. For example, the anti-cancer agent described herein can be formulated as a ready to use solution for parenteral administration. In other examples, the anti-cancer agent described herein can be formulated as a powder (e.g., lyophilized powder) that can be resuspended in a liquid suitable for parenteral administration. In one embodiment, the anti-cancer agent described herein can be formulated for intravenous administration.

The CD44-modulating polypeptide described herein or the anti-cancer described herein can be provided as controlled release pharmaceutical products, which have a goal of improving drug therapy over that achieved by their non controlled counterparts. Controlled release formulations can extend activity of the drug, reduce dosage frequency, and increase subject compliance. In addition, controlled release formulations can be used to affect the time of onset of action or other characteristics, such as blood levels of the drug, and can thus affect the occurrence of side (e.g., adverse) effects. The CD44-modulating polypeptide can be a polypeptide of SEQ ID NO:1 or SEQ ID NO:2

The CD44-modulating polypeptide described herein and an anti-cancer agent described herein can be provided as a pharmaceutical composition. Such pharmaceutical compositions can, as described above, be optionally formulated as a single administration unit or as individual units for administration of each agent. The CD44-modulating polypeptide described herein and the anti-cancer described herein can be provided as part of a kit. Such kits can, for example, improve patient compliance or improve the accuracy or ease of preparation for administering the combination. The kit includes a CD44-modulating polypeptide described herein and an anti-cancer agent where the polypeptide and anti-cancer agent are supplied in a formulation as described herein. The kit can include a package insert or other information (e.g., prescribing information) useful for administration of the combination to a patient in need thereof, such as a cancer patient described herein. The CD44-modulating polypeptide can be a polypeptide of SEQ ID NO:1 or SEQ ID NO:2.

Kits of the invention can include the CD44-modulating polypeptide described herein and an anti-cancer agent described herein having the same or different formulation. Other kits contemplated herein can comprise an agent for the administration of a radiation therapy such as a radioactive isotope. Such other agents for radiation therapy can be provided solely with the CD44-modulating polypeptide or in combination with the CD44-modulating polypeptide and one or more anti-cancer agents described herein. Each component described herein in a kit can be supplied in a separate, individual container. Alternatively or additionally, each component in a kit described herein can be supplied in a single container. In such instances, the container can be a container that can be ready for administration to a patient in need thereof, such as for example, an IV bag, ampoule, a transdermal system (or patch), or a syringe. The CD44-modulating polypeptide can be a polypeptide of SEQ ID NO:1 or SEQ ID NO:2.

The contents of kits described herein can be provided in sterile form. The kit components can come ready-to-use. The kit and its contents can be provided in a form that can be ready for administration to the subject in need. In such instances, the components of the kit are supplied as a formulation and optionally in an administration device such that administration requires little to no further action by the user. Where kits include administration devices, such devices include devices known and understood by those skilled in the art for routes of administration described herein, such as but not limited to, syringes, pumps, bags, cups, inhalers, droppers, patches, creams, atomizers, or injectors.

The pharmaceutical compositions and kits described herein are useful for treating cancers described herein.

The combination therapies described herein can be administered in a regimen. The regimen can be structured to provide therapeutically effective amounts of a CD44-modulating polypeptide described herein, an anti-cancer agent described herein, a radiation therapy described herein, or a combination thereof over a predetermined period of time (e.g., an administration time). The regimen can be structured to limit or prevent side-effects or undesired complications of each of or any combination of the CD44-modulating polypeptide described herein, anti-cancer agent, or radiation therapy described herein. The regimen can be structured in a manner that results in increased effect for the CD44-modulating polypeptide described herein, the anti-cancer agent described herein (e.g., synergy), the radiation therapy, or a combination thereof. Regimens useful for treating cancer can include any number of days of administration which can be repeated as necessary, such as those described herein.

Administration periods can be broken by a rest period that includes no administration of at least one therapy. For example, a regimen can include administration periods that include 2, 3, 5, 7, 10, 15, 21, 28, or more days. In one example, a regimen can include transdermal administration of a CD44-modulating polypeptide described herein. Such transdermal administration can be performed daily (e.g. QD or BID). In another example, a regimen can include parenteral injection of a CD44-modulating polypeptide (e.g. pen injector). Such injections can be performed QD, BID, Q2D, Q3D, or QW. In another example, a regimen can include intranasal administration of a CD44-modulating polypeptide as provided herein. In certain instances a CD44-modulating polypeptide can be administered continuously, without a rest period, during the course of a regimen described herein. In further embodiments, a CD44-modulating polypeptide can be administered as a maintenance therapy after treatment with a regimen described herein. The CD44-modulating polypeptide can be a polypeptide of SEQ ID NO:1 or SEQ ID NO:2.

An anti-cancer agent can be administered as described herein in a regimen that includes a 7, 14, 21, or 28 day cycle. The cycle can include a rest period between cycles. In one example, an anti-cancer agent can be administered in a cycle of 21 days following by a 7 day rest period. Such cycles can be repeated 2, 3, 4, 5, 6, 7, 8, 9, 10, 11, 12, or more times.

Radiation therapy can be administered as described herein in, for example, a regimen that includes a 3, 5, 7, 14, 21, or 28 day cycle. The cycle can include a rest period between cycles. In one example, an anti-cancer agent can be administered in a cycle of 5 days following by a 2 day rest period. Such cycles can be repeated 2, 3, 4, 5, 6, 7, 8, 9, 10, 11, 12, or more times.

Regimens can include a rest period of at least 1, 2, 3, 5, 7, 10, or more days, where at least one therapy is no longer administered to a patient. The rest period can be determined by, for example, monitoring the reaction of the patient to the drug or by measuring the efficacy of the treatment. A rest period can be applicable to a single therapy, such that only one therapy of the CD44-modulating polypeptide described herein, anti-cancer agent described herein, or radiation therapy is discontinued in the rest period but the other therapy(ies) are still administered. Rest periods can be applied to all of the therapies administered to the subject such that the subject receives no therapy for a set period of time during the rest period. In certain instances, the CD44-modulating polypeptide described herein can be administered daily without interruption. The CD44-modulating polypeptide can be a polypeptide of SEQ ID NO:1 or SEQ ID NO:2.

Regimens described herein for the treatment of cancer using the combinations described herein can be continued until disease progression or unacceptable toxicity.

Regimens for administration of combinations described herein include, for example administration of an anti-cancer agent or radiation therapy described herein BIW or TIW and administration of a PD-1 inhibitor. For example, an anti-cancer agent described herein can be administered QD for about 21 days and a CD44-modulating polypeptide described herein can be administered QD. For example, an anti-cancer agent described herein can be administered QD and a CD44-modulating polypeptide described herein can be administered QD via transdermal administration as described herein. In another example, regimens for administration of combinations described herein include administration of radiation therapy QD and a CD44-modulating polypeptide described herein can be administered QD. In still another example, radiation therapy can be administered QD and a CD44-modulating polypeptide described herein can be administered QD via transdermal administration as described herein.

For example, an anti-cancer agent described herein can be administered QD for about 21 days and a CD44-modulating polypeptide described herein can be administered BID. For example, an anti-cancer agent described herein can be administered QD and a CD44-modulating polypeptide described herein can be administered BID via transdermal administration as described herein. In another example, radiation therapy can be administered QD for 5 consecutive days and a CD44-modulating polypeptide described herein can be administered BID. In still another example, radiation therapy can be administered QD and a CD44-modulating polypeptide described herein can be administered BID via transdermal administration as described herein. The CD44-modulating polypeptide can be a polypeptide of SEQ ID NO:1 or SEQ ID NO:2.

For example, an anti-cancer agent described herein or radiation therapy described herein can be administered or BIW or TIW for about 21 days and a CD44-modulating polypeptide described herein can be administered QD. The CD44-modulating polypeptide can be a polypeptide of SEQ ID NO:1 or SEQ ID NO:2. For example, an anti-cancer agent or radiation therapy described herein can be administered or BIW or TIW for about 21 days and a CD44-modulating polypeptide described herein can be administered QD via transdermal administration as described herein.

For example, an anti-cancer agent described herein or radiation therapy described herein can be administered BIW and a CD44-modulating polypeptide described herein can be administered QD. For example, an anti-cancer agent described herein or radiation therapy described herein can be administered BIW and a CD44-modulating polypeptide described herein can be administered QD via transdermal administration as described herein. The CD44-modulating polypeptide can be a polypeptide of SEQ ID NO:1 or SEQ ID NO:2.

For example, an anti-cancer agent described herein or radiation therapy described herein can be administered BIW and a CD44-modulating polypeptide described herein can be administered BID. For example, an anti-cancer agent described herein or radiation therapy described herein can be administered BIW and a CD44-modulating polypeptide described herein can be administered BID via transdermal administration as described herein. The CD44-modulating polypeptide can be a polypeptide of SEQ ID NO:1 or SEQ ID NO:2.

For example, an anti-cancer agent described herein or radiation therapy described herein can be administered TIW and a CD44-modulating polypeptide described herein can be administered QD. For example, an anti-cancer agent or radiation therapy described herein described herein can be administered TIW and a CD44-modulating polypeptide described herein can be administered QD via transdermal administration as described herein. The CD44-modulating polypeptide can be a polypeptide of SEQ ID NO:1 or SEQ ID NO:2.

For example, an anti-cancer agent described herein or radiation therapy described herein can be administered. TIW and a CD44-modulating polypeptide described herein can be administered BID. For example, an anti-cancer agent described herein or radiation therapy described herein can be administered TIW and a CD44-modulating polypeptide described herein can be administered BID via transdermal administration as described herein. The CD44-modulating polypeptide can be a polypeptide of SEQ ID NO:1 or SEQ ID NO:2.

For example, an anti-cancer agent described herein or radiation therapy described herein can be administered QW and a CD44-modulating polypeptide described herein can be administered QD. For example, an anti-cancer agent described herein or radiation therapy described herein can be administered QW and a CD44-modulating polypeptide described herein can be administered QD via transdermal administration as described herein. The CD44-modulating polypeptide can be a polypeptide of SEQ ID NO:1 or SEQ ID NO:2.

For example, an anti-cancer agent described herein or radiation therapy described herein can be administered QW and a CD44-modulating polypeptide described herein can be administered BID. For example, an anti-cancer agent described herein or radiation therapy described herein can be administered QW and a CD44-modulating polypeptide described herein can be administered BID via transdermal administration as described herein. The CD44-modulating polypeptide can be a polypeptide of SEQ ID NO:1 or SEQ ID NO:2.

For example, an anti-cancer agent described herein or radiation therapy described herein can be administered Q2W and a CD44-modulating polypeptide described herein can be administered QD. For example, an anti-cancer agent described herein or radiation therapy described herein can be administered Q2W and a CD44-modulating polypeptide described herein can be administered QD via transdermal administration as described herein. The CD44-modulating polypeptide can be a polypeptide of SEQ ID NO:1 or SEQ ID NO:2.

For example, an anti-cancer agent described herein or radiation therapy described herein can be administered Q2W and a CD44-modulating polypeptide described herein can be administered BID. For example, an anti-cancer agent described herein or radiation therapy described herein can be administered Q2W and a CD44-modulating polypeptide described herein can be administered BID via transdermal administration as described herein. The CD44-modulating polypeptide can be a polypeptide of SEQ ID NO:1 or SEQ ID NO:2.

For example, an anti-cancer agent described herein or radiation therapy described herein can be administered Q3W and a CD44-modulating polypeptide described herein can be administered QD. For example, an anti-cancer agent described herein or radiation therapy described herein can be administered Q3W and a CD44-modulating polypeptide described herein can be administered BID. For example, an anti-cancer agent or radiation therapy described herein described herein can be administered Q3W and a CD44-modulating polypeptide described herein can be administered via transdermal administration as described herein. The CD44-modulating polypeptide can be a polypeptide of SEQ ID NO:1 or SEQ ID NO:2.

In one embodiment, a CD44-modulating polypeptide can be administered in a combination therapy as described herein to patients with diseases and disorders associated with or characterized by, undesired angiogenesis in combination with additional active ingredients, including, but not limited to, anti-cancer drugs, anti-inflammatories, antihistamines, antibiotics, and steroids. The CD44-modulating polypeptide can be a polypeptide of SEQ ID NO:1 or SEQ ID NO:2.

Patients benefitting from the methods described herein can include patients who have been previously treated for cancer but are non-responsive to standard therapies or radiation therapy. In such instances patients may be non-responsive or have developed resistance to anti-cancer treatment(s) or radiation therapy(ies). Patients may have refractory cancer or cancer that is otherwise non-response to at least one anti-cancer therapy or radiation therapy. A patient may also include those who have not previously been treated (e.g. treatment naive) by administering a combination therapy as described herein. Patients can also include those patients who have undergone surgery in an attempt to treat the disease or condition at issue. The methods and combination therapies described herein are equally applicable to patients who have not undergone surgery prior to administration. Patients currently taking agents for treating cancer (e.g., concurrently chemotherapy, immunotherapy, biologics, or hormonal therapy), or undergoing radiation therapy, can benefit from addition of a CD44-modulating polypeptide described herein to the treatment regimen.

The methods of treating described herein are applicable to all cancer patients regardless of patient's age, although some diseases or disorders are more common in certain age groups. Patients with certain preconditions, having undergone certain medical procedures, or are currently taking certain therapies, in certain instances, can be excluded from the methods described herein.

Because patients with cancer have heterogeneous clinical manifestations and varying clinical outcomes, the treatment given to a patient may vary, depending, in part, on a combination of (1) prognosis, (2) responsiveness to therapy and (3) tolerance to therapy. The present disclosure provides methods of varying treatment using the CD44-modulating polypeptides described herein in combination with an anti-cancer agent or radiation therapy.

The CD44-modulating polypeptides described herein used in combination with one or more anti-cancer agents described herein, radiation therapy as described herein, or a combination thereof, are useful for treating, preventing, and/or managing cancer of the skin tissues, organs, blood, and vessels, including, but not limited to, cancers of the bladder, bone, blood, brain, breast, cervix, chest, colon, endrometrium, esophagus, eye, head and neck, kidney, liver, lymph nodes, lung, mouth, ovaries, pancreas, peritoneal, prostate, rectum, stomach, testis, throat, and uterus. In one embodiment, cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, follicular lymphoma, low grade follicular lymphoma, acute myelogenous leukemia, acute lymphocytic leukemia, chronic myelogenous leukemia, chronic lymphocytic leukemia, malignant melanoma, malignant mesothelioma, malignant pleural effusion mesothelioma syndrome, peritoneal carcinoma, primary peritoneal cancer, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. The CD44-modulating polypeptide can be a polypeptide of SEQ ID NO:1 or SEQ ID NO:2.

In another example the combination of a CD44-modulating polypeptide described herein and an anti-cancer agent described herein, radiation therapy described herein, or combination thereof, can be useful for treating cancer of the bladder, brain, breast, colon, endrometrium, head and neck, kidney, liver, lung, ovaries, pancreas, primary peritoneal, prostate, rectum, stomach, testis, throat, and uterus. In another example, the cancer can be breast cancer, colon cancer, ovarian cancer, pancreatic cancer, peritoneal cancer or prostate cancer. The CD44-modulating polypeptide can be a polypeptide of SEQ ID NO:1 or SEQ ID NO:2. In still another example the combination of a CD44-modulating polypeptide described herein and an anti-cancer agent described herein, radiation therapy described herein, or combination thereof, can be useful for treating ovarian cancer, breast cancer, colorectal cancer, prostate cancer, head and neck cancer, endometrial cancer, primary peritoneal cancers, liver cancer, or glioblastoma. In yet another example the combination of a CD44-modulating polypeptide described herein and an anti-cancer agent described herein, radiation therapy described herein, or combination thereof, can be useful for treating ovarian cancer, breast cancer, colorectal cancer, prostate cancer, head and neck cancer, endometrial cancer, primary peritoneal cancers, or glioblastoma. In one embodiment, the cancer is liver cancer. In one embodiment, the cancer is breast cancer. In one embodiment, the cancer is ovarian cancer. In one embodiment, the cancer is glioblastoma. In one embodiment, the cancer is glioblastoma and is treated according to the methods described herein where the treatment comprises administering a CD44-modulating peptide described herein (e.g. SEQ ID NO:1) in combination with radiation therapy as described herein. In one embodiment, the cancer is liver cancer and is treated according to the methods described herein where the treatment comprises administering a CD44-modulating peptide described herein (e.g. SEQ ID NO:1) in combination with radiation therapy as described herein.

The cancer can be a solid tumor. The cancer can be a solid tumor selected from the group consisting of squamous cell carcinoma, nonsquamous cell carcinoma, non-small cell lung cancer (NSCLC), small cell lung cancer, renal cell carcinoma, ovarian cancer, breast cancer, prostate cancer, head and neck cancer, endometrial cancer, primary peritoneal cancers, glioblastoma, colorectal cancer, or pancreatic cancer. In one embodiment, a CD44-modulating polypeptide can be administered in combination with an anti-cancer agent described herein, radiation therapy described herein, or combination thereof, for treating breast cancer, ovarian cancer, or endometrial cancer. In another embodiment, a CD44-modulating polypeptide can be administered in combination with an anti-cancer agent described herein, radiation therapy described herein, or combination thereof, for treating colorectal cancer, peritoneal cancer, or renal cell carcinoma. In another embodiment, a CD44-modulating polypeptide can be administered to treat NSCLC or small cell lung cancer.

The cancer can be a hematological cancer. The hematological cancer can be leukemia, such as for example AML. In one embodiment, a CD44-modulating polypeptide can be administered in combination with an anti-cancer agent described herein for treating AML.

A CD44-modulating polypeptide can bind to CD44 and inhibit activity of an ABC transporter. In one example the polypeptide of SEQ ID NO:1 can bind to CD44 and inhibit activity of an ABC transporter. In another example the polypeptide of SEQ ID NO:2 can bind to CD44 and inhibit activity of an ABC transporter. The ABC transporter can be involved in mechanisms of cancer cells to eliminate anti-cancer agents, or mechanisms of cancer cells to survive radiation therapy. For example, the ABC transporter inhibited by a CD44-modulating polypeptide described herein can eliminate PARP inhibitors from the cell. In another example, the ABC transporter inhibited by a CD44-modulating polypeptide described herein eliminates taxanes from the cancer cell. In another example, the ABC transporter inhibited by a CD44-modulating polypeptide described herein eliminates platinum agents from the cancer cell. In another example, the ABC transporter eliminates anthracyclin agents from the cancer cell. Elimination of anti-cancer agents can decrease the efficacy of such anti-cancer agents, promote resistance. Without being bound by any particular theory, administration of a CD44-modulating polypeptide described herein in combination with an anti-cancer agent described herein, radiation therapy described herein, or combination thereof can increase the efficacy of both agents by inhibiting ABC transporters and its capability to eliminate one or more anti-cancer agents). The ABC transporter inhibited by the CD44-modulating polypeptide described herein can be an ABC transporter set forth in Table 1.

TABLE 1
ABC Transporters:

| | | | |
|---|---|---|---|
| ABC | | Cancer-related | Expression in cancer stem cell- |
| family | Chemotherapy substrates* | cellular substrates | like populations |

ABCA

| | | | |
|---|---|---|---|
| ABCA1 | ND | S1P and cholesterol | ND |
| ABCA2 | Estramustine and mitoxantrone | Cholesterol | Lung cancer cell lines and AML |
| ABCA3 | Anthracycline | Phospholipids | Neuroblastoma |

ABCB

| | | | |
|---|---|---|---|
| ABCB1 | Colchicine, anthracyclines, | PAF | AML and lung cancer cell lines |
| | epipodophyllotoxins, vinca alkaloids, | | |
| | taxanes, camptothecins, bisantrene, | | |
| | imatinib, mitoxantrone, saquinivir, | | |
| | methotrexate and actinomycin D | | |
| ABCB4 | Anthracyclines, vinca alkaloids, taxanes, | ND | ND |
| | epipodophyllotoxins and mitoxantrone | | |
| ABCB5 | Anthracyclines, camptothecins and | ND | Melanoma |
| | thiopurines | | |
| ABCB11 | Taxanes | ND | ND |

ABCC

| | | | |
|---|---|---|---|
| ABCC1^{§} | Anthracyclines, mitoxantrone, vinca | LTC₄, PGA₂, 15d- | Squamous cell carcinoma lines, lung |
| | alkaloids, imatinib, epipodophyllotoxins, | PGJ₂, PGE₂ and S1P | cancer cell lines, glioma and AML |
| | camptothecins, colchicine, saquinivir and | | |
| | methotrexate | | |
| ABCC2^{§} | Vinca alkaloids, cisplatin, taxanes, | LTC₄, PGD₂, | ND |
| | anthracyclines, methotrexate, | PGA₁and PGE₂ | |
| | epipodophyllotoxins, camptothecins, | | |
| | mitoxantrone and saquinivir | | |
| ABCC3^{§} | Methotrexate and epipodophyllotoxins | LTC₄, and 15d-PGJ₂ | ND |
| ABCC4^{§} | Thiopurines, PMEA, methotrexate, AZT | LTB₄, LTC₄, PGA₁, | ND |
| | and camptothecins | PGE₁, PGE₂, PGF₁₀, | |
| | | PGF₂₀, TXB₂, cAMP | |
| | | and cGMP | |
| ABCC5 | Thiopurines, methotrexate, cisplatin, | cAMP and cGMP | ND |
| | PMEA and AZT | | |
| ABCC6 | Anthracyclines, cisplatin and | LTC₄ | ND |
| | epipodophyllotoxins | | |
| ABCC10 | Vinca alkaloids and taxanes | LTC₄ | ND |
| ABCC11 | Thiopurines | LTC₄, cAMP and | ND |
| | | cGMP | |

ABCG

| | | | |
|---|---|---|---|
| ABCG2 | Mitoxantrone, camptothecins, | cGMP | Lung cancer, AML, oesophageal |
| | anthracydins, bisantrene, imatinib, | | carcinoma, glioma, neuroblastoma, |
| | methotrexate, flavopiridol and | | squamous cell carcinoma cell lines, |
| | epipodophyllotoxins | | melanoma, ovarian cancer and |
| | | | nasopharyngeal carcinoma cell lines |

In another embodiment, the CD44-modulating polypeptide binds to CD44 and inhibits co-receptor interaction with CD44. In such instances, the co-receptor can be a receptor tyrosine kinase (RTK) as described herein, such as for example, VEGFR.

Provided herein is a method of treating locally advanced or metastatic transitional cell bladder cancer in a patient in need thereof by administering a CD44-modulating polypeptide described herein in combination with gemcitabine and cisplatinum. The combination can, in certain embodiments, further include radiation therapy as described herein. The CD44-modulating polypeptide can be a polypeptide of SEQ ID NO:1 or SEQ ID NO:2.

Provided herein is a method of treating pediatric patients with relapsed or progressive brain tumors or recurrent neuroblastoma by administering a CD44-modulating polypeptide described herein in combination with temozolomide. The combination can, in certain embodiments, further include radiation therapy as described herein. The CD44-modulating polypeptide can be a polypeptide of SEQ ID NO:1 or SEQ ID NO:2.

Provided herein is a method of treating patients with glioblastoma, by administering a CD44-modulating polypeptide described herein in combination with radiation therapy as described herein. Provided herein is a method of treating patients with recurrent glioblastoma, by administering a CD44-modulating polypeptide described herein in combination with irinotecan. The combination can, in certain embodiments, further include radiation therapy as described herein. The CD44-modulating polypeptide can be a polypeptide of SEQ ID NO:1 or SEQ ID NO:2.

In another aspect is a method for treating pediatric patients with brain stem glioma by administering a CD44-modulating polypeptide described herein in combination with carboplatin. The combination can, in certain embodiments, further include radiation therapy as described herein. The CD44-modulating polypeptide can be a polypeptide of SEQ ID NO:1 or SEQ ID NO:2.

In another aspect is a method for treating pediatric patients with progressive malignant gliomas by administering a CD44-modulating polypeptide described herein in combination with procarbazine. The combination can, in certain embodiments, further include radiation therapy as described herein. The CD44-modulating polypeptide can be a polypeptide of SEQ ID NO:1 or SEQ ID NO:2.

In another aspect is a method for treating patients with poor prognosis malignant brain tumors, newly diagnosed or recurrent glioblastoma multiforms by administering a CD44-modulating polypeptide described herein in combination with cyclophosphamide. The combination can, in certain embodiments, further include radiation therapy as described herein. The CD44-modulating polypeptide can be a polypeptide of SEQ ID NO:1 or SEQ ID NO:2

In another aspect is a method for treating high grade recurrent malignant gliomas by administering a CD44-modulating polypeptide described herein in combination with Gliadel®, The combination can, in certain embodiments, further include radiation therapy as described herein. The CD44-modulating polypeptide can be a polypeptide of SEQ ID NO:1 or SEQ ID NO:2.

In another aspect is a method for treating anaplastic astrocytoma by administering a CD44-modulating polypeptide described herein in combination with temozolomide and tamoxifen. The combination can, in certain embodiments, further include radiation therapy as described herein. The CD44-modulating polypeptide can be a polypeptide of SEQ ID NO:1 or SEQ ID NO:2.

In another aspect is a method for treating gliomas, glioblastoma, anaplastic astrocytoma or anaplastic oligodendroglioma by administering a CD44-modulating polypeptide described herein in combination with topotecan. The combination can, in certain embodiments, further include radiation therapy as described herein. The CD44-modulating polypeptide can be a polypeptide of SEQ ID NO:1 or SEQ ID NO:2.

In another aspect is a method for treating patients with ovarian cancer by administering a CD44-modulating polypeptide described herein in combination with olaparib. The combination can, in certain embodiments, further include radiation therapy as described herein. In certain instances the patient has undergone 1, 2, 3, or more previous treatments. The ovarian cancer can be BRCA-mutated ovarian cancer. The CD44-modulating polypeptide can be a polypeptide of SEQ ID NO:1 or SEQ ID NO:2.

In another aspect is a method for treating patients with metastatic breast cancer by administering a CD44-modulating polypeptide described herein in combination with methotrexate, cyclophosphamide, taxane, abraxane, lapatinib, herceptin, aromatase inhibitors, selective estrogen modulators, estrogen receptor antagonists, and/or PLX3397 (Plexxikon). The combination can, in certain embodiments, further include radiation therapy as described herein. In one example, the method includes administering a CD44-modulating polypeptide described herein in combination with methotrexate, cyclophosphamide, lapatinib, herceptin, aromatase inhibitors, selective estrogen modulators, estrogen receptor antagonists, and/or PLX3397 (Plexxikon). The combination can, in certain embodiments, further include radiation therapy as described herein. The CD44-modulating polypeptide can be a polypeptide of SEQ ID NO:1 or SEQ ID NO:2.

In another aspect is a method for treating patients with pancreatic cancer by administering a CD44-modulating polypeptide described herein in combination with gemcitabine. The combination can, in certain embodiments, further include radiation therapy as described herein. The CD44-modulating polypeptide can be a polypeptide of SEQ ID NO:1 or SEQ ID NO:2.

In another aspect is a method for treating patients with colon cancer by administering a CD44-modulating polypeptide described herein in combination ARISA®, avastatin, taxol, and/or taxotere. The combination can, in certain embodiments, further include radiation therapy as described herein. The CD44-modulating polypeptide can be a polypeptide of SEQ ID NO:1 or SEQ ID NO:2.

In another aspect is a method for treating patients with refractory colorectal cancer or patients who fail first line therapy or have poor performance in colon or rectal adenocarcinoma by administering a CD44-modulating polypeptide described herein in combination with capecitabine and/or PLX4032 (Plexxikon). The combination can, in certain embodiments, further include radiation therapy as described herein. The CD44-modulating polypeptide can be a polypeptide of SEQ ID NO:1 or SEQ ID NO:2.

In another aspect is a method for treating refractory colorectal cancer by administering a CD44-modulating polypeptide described herein in combination with capecitabine, xeloda, and/or CPT-11. The combination can, in certain embodiments, further include radiation therapy as described herein. The CD44-modulating polypeptide can be a polypeptide of SEQ ID NO:1 or SEQ ID NO:2

In another aspect is a method for treating patients with refractory colorectal cancer or patients with unresectable or metastatic colorectal carcinoma by administering a CD44-modulating polypeptide described herein in combination with capecitabine and irinotecan. The combination can, in certain embodiments, further include radiation therapy as described herein. The CD44-modulating polypeptide can be a polypeptide of SEQ ID NO:1 or SEQ ID NO:2.

In another aspect is a method for treating patients with unresectable or metastatic hepatocellular carcinoma by administering a CD44-modulating polypeptide described herein in combination with interferon alpha or capecitabin. The combination can, in certain embodiments, further include radiation therapy as described herein. The CD44-modulating polypeptide can be a polypeptide of SEQ ID NO:1 or SEQ ID NO:2.

In another aspect is a method for treating patients with refractory or relapsed or high-risk acute myelogenous leukemia by administering a CD44-modulating polypeptide described herein in combination with fludarabine, carboplatin, and/or topotecan. The combination can, in certain embodiments, further include radiation therapy as described herein. The CD44-modulating polypeptide can be a polypeptide of SEQ ID NO:1 or SEQ ID NO:2.

In another aspect is a method for treating patients with unfavorable karotype acute myeloblastic leukemia by administering a CD44-modulating polypeptide described herein in combination with liposomal daunorubicin, topotecan and/or cytarabine. The combination can, in certain embodiments, further include radiation therapy as described herein. The CD44-modulating polypeptide can be a polypeptide of SEQ ID NO:1 or SEQ ID NO:2.

In another aspect is a method for treating patients with non-small cell lung cancer by administering a CD44-modulating polypeptide described herein in combination with gemcitabine, abraxane, erlotinib, geftinib, and/or irinotecan. The combination can, in certain embodiments, further include radiation therapy as described herein. The CD44-modulating polypeptide can be a polypeptide of SEQ ID NO:1 or SEQ ID NO:2.

In another aspect is a method for treating patients with non-small cell lung cancer by administering a CD44-modulating polypeptide described herein in combination with carboplatin and irinotecan. The combination can, in certain embodiments, further include radiation therapy as described herein. The CD44-modulating polypeptide can be a polypeptide of SEQ ID NO:1 or SEQ ID NO:2.

In another aspect is a method for treating patients with non-small cell lung cancer who have been previously treated with carbo/VP 16 and radiotherapy by administering a CD44-modulating polypeptide described herein in combination with doxetaxol. The combination can, in certain embodiments, further include radiation therapy as described herein. The CD44-modulating polypeptide can be a polypeptide of SEQ ID NO:1 or SEQ ID NO:2.

In another aspect is a method for treating patients with non-small cell lung cancer by administering a CD44-modulating polypeptide described herein in combination with carboplatin, paclitaxel and/or thoracic radiotherapy. The CD44-modulating polypeptide can be a polypeptide of SEQ ID NO:1 or SEQ ID NO:2

In another aspect is a method for treating patients with stage IIIB or IV non-small cell lung cancer by administering a CD44-modulating polypeptide described herein in combination with taxotere. The combination can, in certain embodiments, further include radiation therapy as described herein. The CD44-modulating polypeptide can be a polypeptide of SEQ ID NO:1 or SEQ ID NO:2.

In another aspect is a method for treating patients with small cell lung cancer by administering a CD44-modulating polypeptide described herein in combination with oblimersen. The combination can, in certain embodiments, further include radiation therapy as described herein. The CD44-modulating polypeptide can be a polypeptide of SEQ ID NO:1 or SEQ ID NO:2.

In another aspect is a method for treating patients with lymphoma and other blood cancers by administering a CD44-modulating polypeptide described herein in combination with ABT-737 and/or obatoclax. The combination can, in certain embodiments, further include radiation therapy as described herein. The CD44-modulating polypeptide can be a polypeptide of SEQ ID NO:1 or SEQ ID NO:2.

In another aspect is a method for treating patients with various types of lymphoma by administering a CD44-modulating polypeptide described herein in combination with vinblastine or fludarabin. The combination can, in certain embodiments, further include radiation therapy as described herein. The CD44-modulating polypeptide can be a polypeptide of SEQ ID NO:1 or SEQ ID NO:2.

In another aspect is a method for treating patients with ovarian cancer by administering a CD44-modulating polypeptide described herein in combination with taxol, carboplatin, doxorubicin, gemcitabine, cisplatin, xeloda, paclitaxel, dexamethasone, or a combination thereof. The combination can, in certain embodiments, further include radiation therapy as described herein. The CD44-modulating polypeptide can be a polypeptide of SEQ ID NO:1 or SEQ ID NO:2.

In another aspect is a method for treating patients with prostate cancer by administering a CD44-modulating polypeptide described herein in combination with xeloda, 5 FU/LV, gemcitabine, irinotecan plus gemcitabine, cyclophosphamide, vincristine, dexamethasone, GM-CSF, celecoxib, taxotere, ganciclovir, paclitaxel, adriamycin, docetaxel, estramustine, Emcyt, denderon or a combination thereof. The combination can, in certain embodiments, further include radiation therapy as described herein. The CD44-modulating polypeptide can be a polypeptide of SEQ ID NO:1 or SEQ ID NO:2.

In another aspect is a method for treating patients with renal cell cancer by administering a CD44-modulating polypeptide described herein in combination with capecitabine, tamoxifen, IL-2, GM-CSF, Celebrex®, or a combination thereof. The combination can, in certain embodiments, further include radiation therapy as described herein. The CD44-modulating polypeptide can be a polypeptide of SEQ ID NO:1 or SEQ ID NO:2.

In another aspect is a method for treating patients with gynecologic, uterus or soft tissue sarcoma cancer by administering a CD44-modulating polypeptide described herein in combination with IFN, a COX-2 inhibitor, and/or sulindac. The combination can, in certain embodiments, further include radiation therapy as described herein. The CD44-modulating polypeptide can be a polypeptide of SEQ ID NO:1 or SEQ ID NO:2.

In another aspect is a method for treating patients with a solid tumor by administering a CD44-modulating polypeptide described herein in combination with celebrex, etoposide, cyclophosphamide, docetaxel, apecitabine, IFN, tamoxifen, IL-2, GM-CSF, or a combination thereof. The combination can, in certain embodiments, further include radiation therapy as described herein. The CD44-modulating polypeptide can be a polypeptide of SEQ ID NO:1 or SEQ ID NO:2.

It is understood that modifications which do not substantially affect the activity of the various embodiments of this invention are also included within the definition of the invention provided herein. Accordingly, the following examples are intended to illustrate but not limit the present invention.

### EXAMPLES

### Example 1

A6 shares sequence homology with the Link Module of CD44. The Link Module of CD44 has been shown to be critical to Hyaluronan (HA) binding and cell migration. When the CD44 Link Module was substituted with a homologous region of higher HA affinity (TSG-6), cells expressing this chimera bound HA, but failed to migrate and were described as tethered. A6 was shown to increase the binding of CD44-expressing SKOV3 cells to HA coated plates. This effect was blocked with the anti-CD44 antibody, IM7. However, neither A6 nor IM7 had any effect on the binding of CD44-nonexpressing A278 cells to HA coated plates. These results suggest that increasing adhesion may play a role in the antimetastatic activity of the A6 peptide, and again illustrate correlation of A6 activity with CD44 expression. The study further demonstrated that A6 perturbed the binding of the anti-CD44 antibody, DF1485, to CD44-expressing SKOV3 cells. This was reported to be a partial inhibition which did not result from a competition involving either A6 or CD44. Furthermore, the DF1485 antibody did not recognize A6 or inhibit the binding of an anti-A6 antibody to A6. Without being bound by any particular A6 can induce conformational changes in CD44, resulting in either a lowered affinity of the epitope for DF1485, or preventing DF1485 from binding CD44. The binding of A6 to CD44 results in a modulation of CD44-mediated intracellular signaling and establishes a functional relationship between A6 and CD44 in CD44 expressing cells.

CD44 is a complex multifunctional receptor modulating a variety of cellular processes. Studies described herein demonstrate that A6 inhibits the metastatic process in a CD44 dependent manner. Because CD44 is associated with a chemoresistant and radiation resistant phenotype, which is countered by inhibition of CD44 signaling, A6 is a candidate for inhibition of CD44-mediated resistance. A6 may be used in combination with a cytotoxic chemotherapeutic agent or radiation therapy to inhibit metastases and to render resistant cells sensitive to chemotherapy. Furthermore, due to the positive safety profile documented for A6, there would be a reduced likelihood of compounding toxicity. As such, A6 may be combined with almost any chemotherapeutic or radiation therapy, or a combination of both. This safety profile also invites the use of A6 for longer-term maintenance therapy to prevent recurrence stemming from micrometastases surviving first-line standard of care treatment. A6 has demonstrated activity against CD44 expressing tumor cells and CLL cells, and is a candidate for the treatment of malignant disease and hematological malignancy. A6 has demonstrated clinical safety and efficacy, and by targeting CD44 resistant cells to prevent metastases and recurrence, has the possibility of creating a new paradigm for cancer treatment.

Preclinical studies have shown that A6 has anti-migratory, anti-invasive, and anti-metastatic properties. A6 inhibits migration and invasion of breast, lung, glioma, ovarian, and prostate cancer cell lines in vitro in a dose-dependent manner, and inhibits the growth and metastasis of breast, melanoma, glioma, lung, and prostate cancer cells in xenograft models in vivo. The combination of A6 with tamoxifen resulted in an inhibition of breast tumor cell growth greater than with either A6 or tamoxifen alone. A similar result was observed in glioma xenograft studies where the combination of A6 with cisplatin also inhibited tumor cell growth greater than with either A6 or cisplatin alone. These results are important because of the relationship between CD44 and chemoresistance.

Boyden chamber analyses demonstrated that A6 inhibited chemotaxis in a variety of human breast and ovarian cancer cell lines in a concentration dependent manner. The IC₅₀ for the inhibition of chemotaxis of responsive cell lines was 10-100 nmol/L suggesting physiological relevance. Furthermore, A6 inhibition of chemotaxis was shown to correlate with the expression of CD44. A6 produced more than an 85% inhibition of migration in CD44-positive SKOV3 cells when compared to untreated control. A6 had no effect on the migration of CD44-negative A2780 cells. A6 was also shown to interfere with the binding of only one (DF1485) of the four anti-CD44 antibodies tested. A6 did not interfere with the binding of the anti-CD44 antibody, IM7, which blocks HA binding to CD44. These findings suggest that A6 does not produce a global nonspecific change in CD44, but instead produces a subtle change to a specific epitope.

Because A6 inhibited migration of SKOV3 cells, this study also examined the direct interaction of A6 with CD44. Human ovarian SKOV3 cells were bound and cross-linked to A6. Immunoprecipitation and immunoblotting of lysate preparations of cross-linked cells revealed that A6 was binding to CD44. To determine if this binding influenced CD44-mediated activity, and to determine if a functional relationship existed between A6 and CD44, intracellular signaling studies were conducted. A6 was shown to modulate FAK phosphorylation in CD44-positive SKOV3 cells, but not in CD44-negative A2780 cells. The study further demonstrated that the A6 modulation of FAK phosphorylation in SKOV3 cells was blocked by HA. These results show that a functional relationship exists between A6 and CD44 binding, and CD44-mediated intracellular signaling.

Mammary: The effects of A6 in mammary tumor and metastasis models were investigated. Studies with BALB/c (nu/nu) mice implanted with MDA-MB-231 human mammary carcinoma xenografts demonstrated that A6 inhibited tumor growth by 90% compared to control. An inhibition of metastasis was noted. The effect of A6 in Fisher rats inoculated with Mat B-111 syngeneic mammary carcinoma cells was evaluated. A6 treatment inhibited tumor growth by 55% and markedly suppressed lymph node metastasis. Furthermore, the combination of A6 with tamoxifen in Fisher rats with Mat syngeneic mammary carcinoma resulted in a 75% inhibition of tumor growth.

Prostate: A model of prostate cancer was used to evaluate the antimetastatic effect of A6 in mice. Metastases to lymph nodes were measured following the orthotopic injection of human PC-3M-LN4 prostate cancer cells into the prostates of BALB/c (nu/nu) mice. The percentage of mice with lymph node metastases was reduced from more than 70% in the control group to as low as 22% in A6 treated animals. Additionally, A6 treatment significantly reduced lymph node volume by as much as 70%.

Glioblastoma: In animal models of glioblastoma, U87MG human glioma cells were implanted subcutaneously or intracranially in BALB/c (nu/nu) mice and the animals were divided into different treatment groups. A6 treatment suppressed subcutaneous U87MG tumor growth by 48% and prolonged the time to progression following discontinuation of A6 treatment. In this study the effects of cisplatin were also examined. Cisplatin treatment reduced tumor growth by 53%. Interestingly, the combination of A6 and cisplatin resulted in a 92% inhibition of subcutaneous tumor growth. This result was consistent with a U87MG intracranial xenograft study in which mice receiving a combination of A6 and cisplatin exhibited a significantly greater inhibition of tumor growth (98%) when compared to either A6 (44%) or cisplatin (82%) alone. In this study, the combination therapy also significantly increased survival time over that for either drug alone. This was consistent with subcutaneous xenograft results.

Melanoma: The well characterized B16-F 10 lung metastatic model was employed to determine the ability of A6 to inhibit the colonization of secondary tissues by circulating cancer cells. B16-F10 melanoma cells were evaluated by flow cytometric analysis and were shown to express CD44. The IC₅₀ for A6 inhibition of chemotaxis in B16-F10 cells was 29 nmol/L, indicative of a responsive cell line. Melanoma cells were injected into the tail veins of C57BL/6 mice to simulate a burden of metastasizing cells and the lungs were then evaluated for lesions at day 11. Treatment with A6 reduced the number of lung metastases to 50% of control. Taken with previous results, this is important because it demonstrates that 46 not only inhibits the initial steps of the metastatic process (e.g., migration and invasion), but also inhibits the formation of secondary lesions after tumor cells enter the circulation.

Leukemia: A6 has also been evaluated for activity in hematological malignancies. Chronic lymphocytic leukemia (CLL) is characterized by the accumulation of mature monoclonal B cells in the blood and secondary tissues. CD44 is highly expressed in CLL cells and mediates the interaction between CLL cells and the microenvironment. CLL cells receive survival signals from the microenvironment, and one of these pathways is mediated by CD44. Binding of HA to CD44 has been shown to activate PI3K/AKT and MAPK/ERK mediated survival pathways, and to induce expression of the anti-apoptotic protein Mcl-1, which promotes CLL cell survival. It has been shown that this effect can be blocked by an inhibitor of Mcl-1, or by anti-CD44 monoclonal antibodies, leading to apoptosis in vitro.

Recent studies with human CLL B-cell lymphocytes have shown that A6 down modulates the expression of CD44 and ZAP-70 (a marker for an aggressive form of CLL), and inhibits B-cell receptor (BCR) signaling, resulting in a direct, dose-dependent, cytotoxicity in vitro. To evaluate the effects of A6 in vivo, an established CLL xenograft model was employed. ZAP-70^{pos} B-cell lymphocytes isolated from individual patients were injected into immune-deficient mice treated with A6 or vehicle control. A6 treatment resulted in up to 90% reduction in CLL burden. Previously, A6 had not demonstrated cytotoxicity in solid tumor models of glioma, breast, and ovarian cancer. However, in these CLL studies, A6 was shown to be directly cytotoxic for CLL B-cell lymphocytes. A6 is currently being evaluated for the treatment of CLL.

Several clinical studies have been conducted to evaluate the safety and efficacy of A6. These include safety studies in healthy volunteers as well as studies in patients with varying stages of metastatic disease.

Normal Volunteers: A6 was administered to normal volunteers in a Phase 1a, double-blind, placebo-controlled, parallel-group clinical trial. Results showed there were no systemic drug-related adverse events. No significant alterations in physical examinations, vital signs, electrocardiograms or clinical laboratory testing, including coagulation parameters such as PT, PTT, fibrinogen, and thrombin time, were noted. Pharmacokinetic data in normal volunteers at the 150 mg/day and 300 mg/day single dose levels showed a t_{1/2} of 1.8-2.0 hours at both dose levels. Furthermore, no cumulative increase in concentration over time was detected. Following A6 subcutaneous administration twice daily for 6 days, no anti-A6 antibody production was detected at day 14.

Advanced Gynecologic Cancer: A Phase 1b trial, was conducted in women with advanced gynecologic cancer. Greater than 40% of patients dosed continuously with A6 experienced disease stabilization. The study used a sequential dose-escalation design, with the lowest-dose group (4 patients) receiving A6 for cycles of 14 days “on” followed by 14 days “off”, a regimen not expected to produce any therapeutic effect. Twelve patients with advanced gynecologic malignancies that had failed standard therapy were treated with daily, uninterrupted A6. In this population, in which disease progression is expected, 5 patients (4 of whom had ovarian or primary peritoneal carcinoma) achieved stable tumor measurements for at least 4 months, and 1 for greater than 12 months. Patients continued treatment until disease progression or unacceptable toxicity. Response was evaluated as defined by RECIST and the Gynecologic Cancer Intergroup (GCIG) CA-125 response criteria. A Kaplan-Meier retrospective analysis demonstrated that patients treated with daily A6 showed a delayed time to tumor progression relative to an effective control group, (whose treatment was intermittent and, therefore, not expected to have beneficial effect) providing evidence of antineoplastic activity. Continuous treatment with A6 resulted in an increased time to progression (TTP) with a median TTP of 78 days (95% CI 57.365) compared to 44 days (95% CI 4.62) in patients who received the intermittent therapy (log-rank p-value=0.02). The safety outcome in this Phase 1b gynecologic cancer trial was excellent and showed no specific toxicity profile.

Asymptomatic Progression of Ovarian Cancer: A randomized, double-blind, placebo-controlled Phase 2 clinical trial evaluating A6 in women with asymptomatic CA-125 progression of ovarian cancer (“marker-only relapse” or MOR) was conducted. Patients were in clinical remission after first-line chemotherapy with no evidence of disease following physical examination or imaging analysis, but had two consecutive, above-normal, increases of CA125 (a biomarker for recurrence/poor prognosis). Because patients were clinically asymptomatic at the time of entry, the study was able to be placebo-controlled. The primary endpoints were time to clinical progression of disease and safety of A6. The secondary endpoints included changes in serum CA125. This study enrolled 24 patients: 12 were randomized to daily self-administration of A6 at two doses, and 12 to matching placebo injections. Both groups were followed for up to 9 months. Although there were no complete responses, 36% of patients achieved stable disease. A6 treatment was not associated with CA125 response. Results from a Kaplan-Meier analysis of progression-free survival showed that treatment with A6 significantly prolonged time to progression. Despite the small patient sample size, A6 therapy was associated with a statistically significant increase in progression free survival (PFS) (log-rank p-value=0.01.) with a median PFS of 100 days (95% CI 64.168) compared to 49 days (95% CI 29.67) in patients who received the placebo. Furthermore, the safety profile of A6 was comparable to that of control (placebo) treatment.

Persistent or Recurrent Ovarian Cancer: A Phase 2 trial was conducted in patients with persistent or recurrent epithelial ovarian, fallopian tube, or primary peritoneal carcinoma to evaluate A6 in a patient population with a disease burden greater than that presented in the previously described MOR trial. Patients had received one prior platinum-based chemotherapeutic regimen and were allowed to have received one additional cytotoxic regimen for the management of recurrent or persistent disease. Patients received a 150 mg twice daily subcutaneous dose of A6 and continued on treatment until disease progression or unacceptable toxicity. Response criteria were as defined by RECIST. Primary measures of clinical efficacy were objective tumor response and PFS at 6 months compared to a historical Gynecologic Oncology Group (GOG) dataset based on a similar population of patients. Of the 31 eligible patients evaluated, no responses were observed; 6.5% were progression free for at least 6 months; and 36% of evaluable patients achieved stable disease. A6 was well tolerated but had minimal activity in patients with persistent or recurrent epithelial ovarian, fallopian tube, or primary peritoneal carcinoma under the conditions of this trial. Considering the relationship of A6 to CD44 and the relationship of CD44 to resistant and recurrent disease, it would be of interest to follow this study with a combination trial comparing standard-of-care to standard-of-care plus AG in this difficult population.

A6 has been evaluated for the treatment of ocular disease. The focus of this application has been wet age-related macular degeneration (AMD) and diabetic retinopathy, which are characterized by neovascularization and vascular permeability. Since angiogenesis is known to involve HA and to be mediated by CD44, A6 has been investigated for use as a therapeutic for these conditions. Angiogenesis is a multistage process involving cell migration and extracellular matrix (ECM) remodeling, including the loss of cellular structure and function followed by invasion. Similar cellular changes are also observed early in the metastatic process. These cellular changes can be more accurately described in terms of an epithelial-mesenchymal transition (EMT). EMT is a process by which epithelial cells acquire mesenchymal-like properties, with reduced intercellular adhesion and increased motility, critical to many developmental, homeostatic, and pathological processes. The EMT process is a continuum leading to enhanced cell migration and invasion. Preceding migration, there is a loss of cadherin and epithelial adhesion, followed by disruption of the basement membrane and degradation of the ECM by matrix metalloproteinases (MMPs). A6 has been shown to inhibit this process.

Wet Age-Related Macular Degeneration: Several in vivo studies were conducted to evaluate the efficacy of A6 for the treatment of wet AMD. In the mouse model of laser-induced choroidal neovascularization (CNV), treatment with A6 resulted in a 95% inhibition of new vessel formation compared to the non-treated control group. Results employing a rat model of laser-induced CNV showed that subcutaneous injections of A6 produced a 70% reduction in CNV compared to non-treated controls. Finally, results from a primate model of laser-induced CNV demonstrated that intravitreal administration of A6 resulted in a 71% reduction in CNV relative to control. These studies demonstrate that A6 may be a promising candidate for the treatment of wet AMD.

Diabetic Retinopathy: Research involving the use of A6 for treatment of diabetic retinopathy demonstrated that A6 treatment prevents the loss of vascular endothelial (VE)-cadherin and inhibits the increase in microvascular permeability in the retina of diabetic Brown Norway rats induced with streptozotocin. In the same study, similar results were observed using bovine retinal microvascular endothelial cells and showed that VE-cadherin degradation was associated with increased vascular permeability and the secretion and activation of MMP-2 and MMP-9. Treatment with A6 was shown to inhibit MMP dependent VE-cadherin degradation and the loss of permeability. In addition, A6 prevented the secretion and activation of MM P-2 and MMP-9. HA has also been shown to increase MMP-2 and MMP-9 expression in cell culture and to promote CD44-EGFR interaction leading to MMP-2 secretion and enhanced cell motility. The ability of A6 to inhibit MMP activation may have important implications for the metastatic process.

The role of hepatocyte growth factor (HGF) in angiogenesis, as well as the elevated intravitreous concentrations of HGF in diabetic patients, has been described. The effect of A6 on HGF and its receptor, c-Met, in retinal angiogenesis has been examined. This study demonstrated that HGF was upregulated in the retinas of mice following hypoxia-induced retinal neovascularization. Furthermore, HGF was shown to stimulate retinal microvascular endothelial cell invasion in vitro, which is consistent with the angiogenic process. HGF-induced retinal endothelial cell invasion was reduced to control levels following treatment with A6. Since CD44 functions as a co-receptor with c-Met, these results suggest a possible mechanistic pathway for A6.

The metastatic process involves migration and invasion of tumor cells from the local microenvironment, intravasation into the blood or lymph circulation, extravasation from circulation back into tissue, followed by metastatic colonization and growth or dormancy. Metastasis and recurrence have been linked to a subpopulation of highly invasive tumorigenic cells, which have been shown to be resistant to chemotherapeutics. These tumorigenic cells are characterized by the expression of CD44, a multifunctional receptor involved in cell signaling, adhesion, migration, and proliferation. CD44 functions as a receptor, as a co-receptor (e.g., c-Met and EGFR), and as a platform for MMPs to enable many biological processes. In addition, CD44 is known to mediate invasion and metastasis.

Chemotherapeutic resistance was linked to a number of CD44 pathways including MDRI-dependent efflux of chemotherapeutics. This resistance results in expansion of invasive cells following first-line chemotherapy, which leads to recurrence. Studies have shown that targeting CD44 or related signaling pathways, using RNAi strategies or with anti-CD44 antibodies, will suppress tumor growth and relapse, and increase sensitivity of these cells to chemotherapeutics. In animal xenograft models, A6 has been found to enhance the activities of both tamoxifen to inhibit the growth of breast tumor cell growth, and cisplatin to inhibit the growth of glioma cells. This supports the concept that targeting CD44 may render tumor cells more sensitive to therapeutic agents.

A6 was shown to bind to CD44 and to modulate CD44-mediated activity. A6 demonstrated antimetastatic properties by inhibiting migration and invasion, which are early steps in the metastatic process. The mechanism by which A6 acts may involve inhibition of EMT, as observed in studies of ocular disease where A6 inhibited MMP activation and cadherin degradation. Rationale for this consideration is supported by evidence that HA is implicated in MMP activation. A6 also acts later in the metastatic process to inhibit the formation of lesions resulting from the direct injection of cancer cells into the blood stream. This would indicate that A6 inhibits steps involving extravasation and/or metastatic colonization. This is important when considering recurrence following adjuvant therapy and the possibility of proliferation of dormant micrometastases long term.

Dormancy and micrometastases present a therapeutic challenge. That subclinical micrometastases may be present long term was demonstrated in a study involving 36 breast cancer patients found to be disease free from 8 to 22 years post-resection. This study demonstrated that in one third of these patients, with no evidence of disease, viable circulating tumor cells (CTCs) could be isolated. The CTCs were determined to be non-proliferative with a short half-life, but were found when repeated samples were taken up to 2 years after the patients entered the study. This suggested that CTCs were being continuously released from subclinical micrometastases. Long-term or maintenance therapy targeting recurrence is not practical when considering many cytotoxic agents. However, due to its superior safety profile (no immunogenicity, no dose-limiting toxicities, no serious side-effects), long-term or maintenance therapy with A6 may be an option. The use of A6 in this manner could introduce a new paradigm to cancer treatment.

A6 may act directly on CD44 or by modulating CD44 co-receptor activity, A6 binding to CD44, and the effects of A6 on chemotaxis and intracellular signaling, were demonstrated in the absence of HA. This indicates that A6 has a primary effect, through CAD44, that is independent of HA. However, because A6 increases adhesion of CD44 expressing cells to HA, it also suggests that A6 may interact with HA secondarily to A6 binding to CD44. The CD44 ligand-binding region that shares homology with A6 is likely to be critical to the mechanism by which A6 modulates the activity of CD44. A6 may simulate the CD44 sequence and trigger a homotypic interaction resulting in modification of CD44 activity by inducing a conformational change in CD44, or CD44 dimerization, or both. As mentioned, the perturbation of DF1485 binding by A6 suggests that A6 may induce conformational changes in the receptor. Alternatively, A6 homology may simulate the CD44 sequence permitting it to influence a CD44-binding partner/co-receptor resulting in modulation of CD44-mediated activity. This is supported by A6 inhibitory activity on HGF and MMPs observed in ocular diseases. Finally, although A6 binds to CD44, the possibility cannot be excluded that A6 interacts with a protein independent of CD44 that initiates secondary modulation of CD44 activity.

### Example 2

Analysis of whether A6 differentially modulates the sensitivity of cisplatin-sensitive versus cisplatin resistant human ovarian cancer 2008 cells to cisplatin was tested,

Conditions: Drugs: DDP (3.33 mM stock) and A6 (109.8 mM stock); Drug exposure time: 1 hour ; Cell line: 2008 and its DDP-resistant subline 2008/C31*5.25; Media: RPMI 1640 plus 10% FBS containing 1 mM penicillin/streptomycin; Cytotoxicity Assays: CCK-8 (Cell Counting Kit 8—Dojindo Molecular Technologies).

Tissue culture plates containing 96-well plates were seeded with 3000 cells in 100 μl of media per well. The wells on rows A-D were seeded with 2008 cells, while the wells on rows E-H were seeded with 2008/C13*5.25 cells. The cells were allowed to attach overnight in a 37° C. in 5% CO₂ incubator. Approximately 24 h later medium was removed by aspiration and the cells were exposed to 0, 1, 10 or 100 μM A6 peptide in 100 μl. After 1 h, increasing concentrations of DDP were added to triplicate wells in 100 μl medium. Following a 1 h exposure to the combination of A6 and DDP, the drugs were removed by aspiration. The cells were then allowed to grow for 96 h following which the plates were assayed for relative cell growth with the CCK-8 assay.

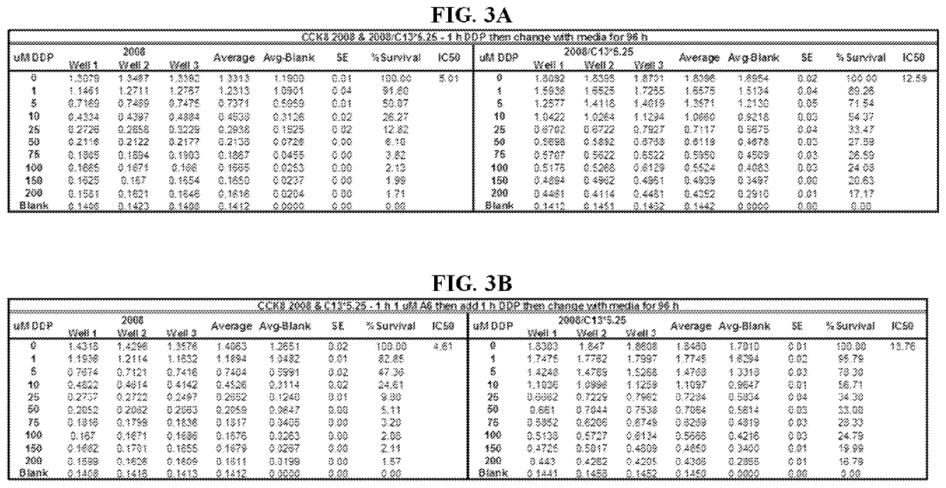

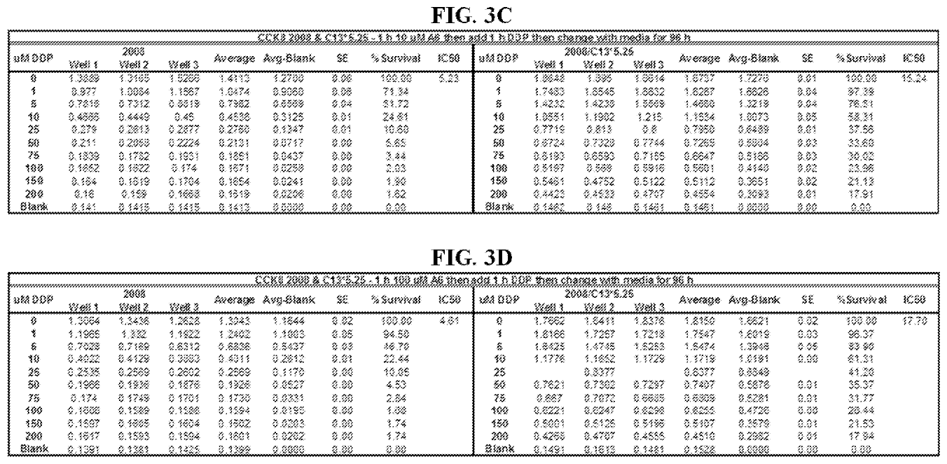

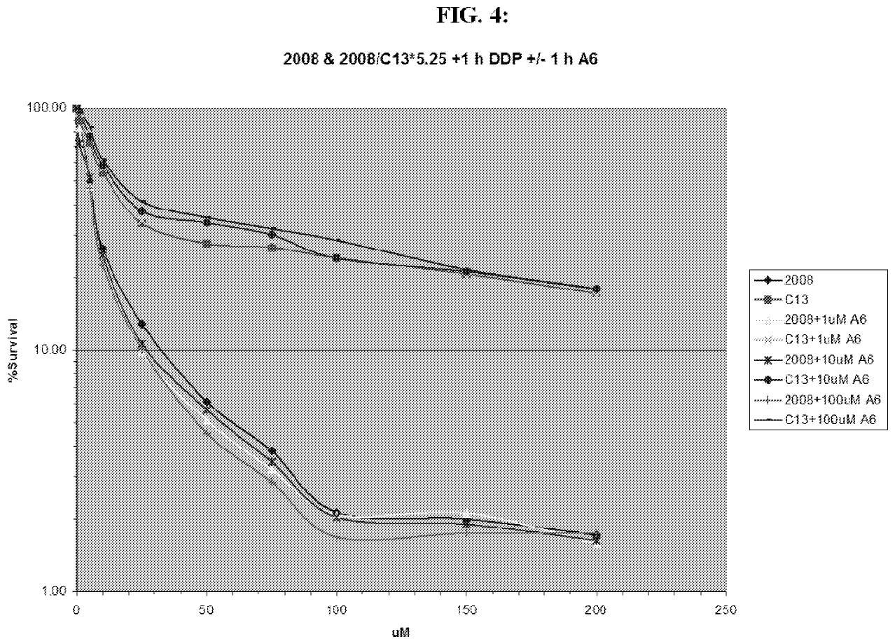

CCK-8 assay. Five μl of CCK-8 stain was added to each well and the plate was incubated at 37° C. incubator for 4 h. The plate was read with a microplate reader at a wavelength of 450 nM. (See FIGS. 3A-3D and FIG. 4)

The assay was performed using triplicate wells for each DDP concentration. The data in the tables below is the optical density in the well which is a function of the number of live cells remaining at the end of the 96 h growth period.

Under conditions where the 2008 cells were growing in RPMI containing 10% FBS, a 1 h exposure to A6 at concentration of 1, 10 or 100 μM followed by a 1 h exposure to both A6 and DDP failed to modulate the sensitivity of either the DDP-sensitive 2008 cells, or the DDP-resistant 2008/C13*5.25 cells to DDP. The concentration of A6 during the first hour of exposure was 1, 10 or 100 μM. During the second hour of exposure, when the DDP was present, the concentration of A6 was 0.5, 5 or 50 μM due to the dilution effect of adding the DDP.

### Example 3

Analysis of whether A6 differentially modulates the sensitivity of cisplatin-sensitive versus cisplatin resistant human ovarian cancer A2780 cells to cisplatin was tested.

Study Conditions. Drugs: DDP (3.33 mM stock) and A6 (109.8 mM stock); Drug exposure time: 1 hour; Cell line: A2780 and its DDP-resistant subline A2780/CP; Media: RPMI 1640 plus 10% FBS containing 1 mM penicillin/streptomycin; Cytotoxicity Assays: CCK-8 (Cell Counting Kit 8—Dojindo Molecular Technologies).

Tissue culture plates containing 96-well plates were seeded with 4000 cells in 100 μl of media per well. The wells on rows A-D were seeded with A2780 cells, while the wells on rows E-H were seeded with A2780/CP cells. The cells were allowed to attach overnight in a 37° C. in 5% CO₂ incubator. Approximately 24 h later medium was removed by aspiration and the cells were exposed to 0, 1, 10 or 100 μM A6 peptide in 100 μl. After 1 h, increasing concentrations of DDP were added to triplicate wells in 100 μl medium. Following a 1 h exposure to the combination of A6 and DDP, the drugs were removed by aspiration. The cells were then allowed to grow for 96 h following which the plates were assayed for relative cell growth with the CCK-8 assay.

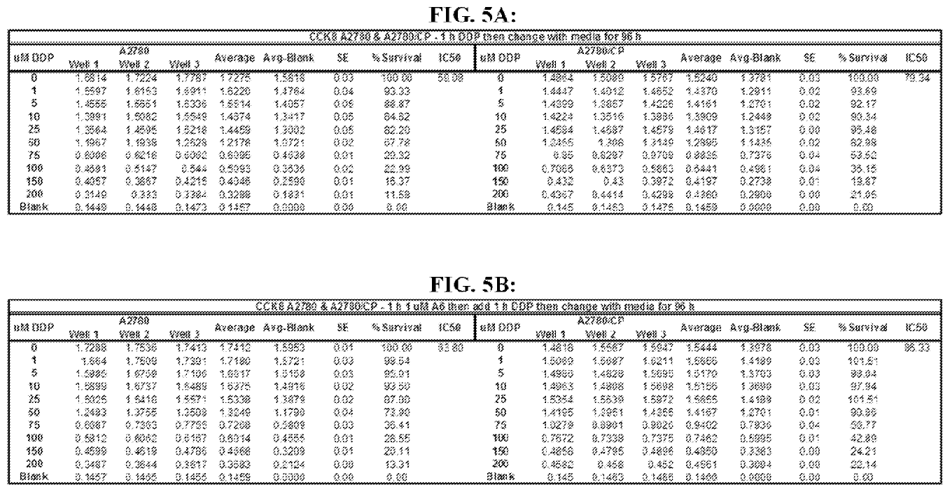

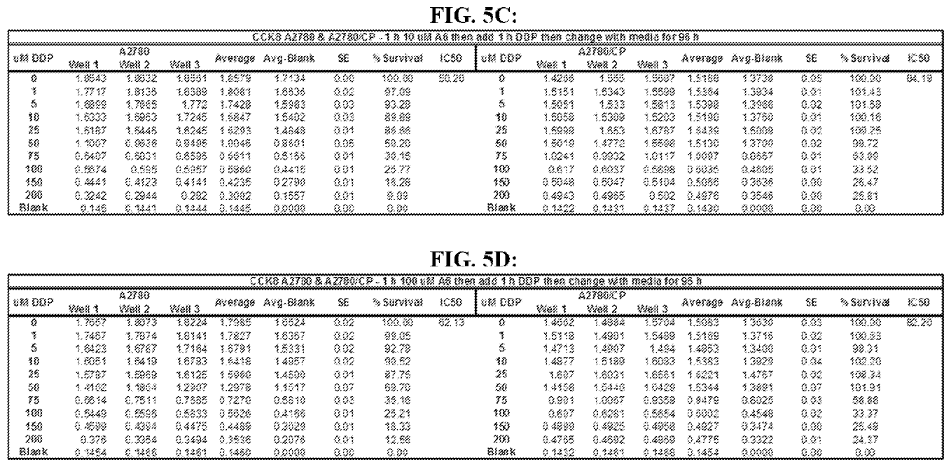

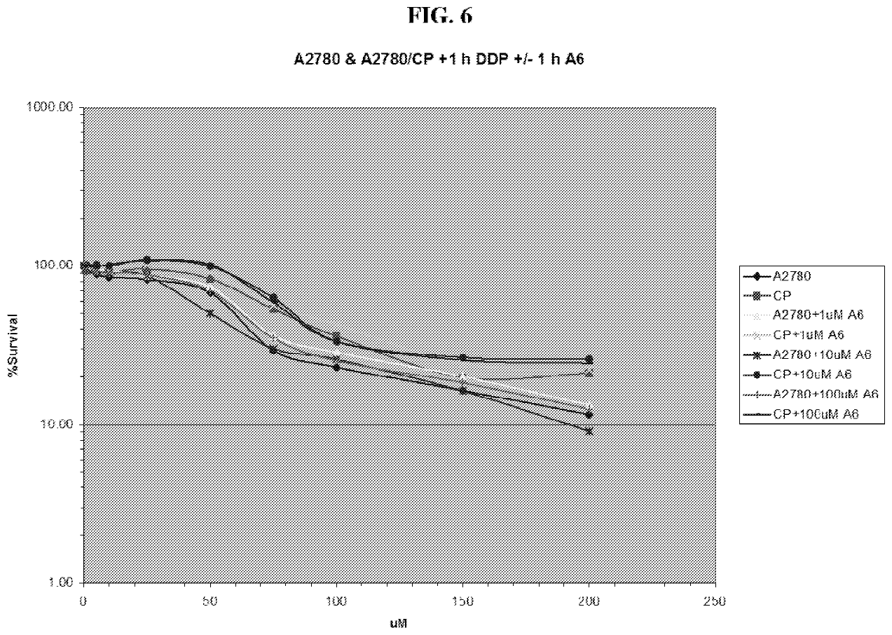

CCK-8 assay. Five μl of CCK-8 stain was added to each well and the plate was incubated at 37° C. incubator for 4 h. The plate was read with a microplate reader at a wavelength of 450 nM. (See FIGS. 5A-5D and FIG. 6).

The assay was performed using triplicate wells for each DDP concentration. The data in the tables below is the optical density in the well which is a function of the number of live cells remaining at the end of the 96 h growth period.

Under conditions where the A2780 cells were growing in RPMI containing 10% FBS, a 1 h exposure to A6 at concentration of 1, 10 or 100 μM followed by a 1 h exposure to both A6 and DDP failed to modulate the sensitivity of either the DDP-sensitive 2008 cells, or the DDP-resistant A2780/CP cells to DDP. In this study the A2780/CP cells were not very resistant to DDP. The concentration of A6 during the first hour of exposure was 1, 10 or 100 μM. During the second hour of exposure, when the DDP was present, the concentration of A6 was 0.5, 5 or 50 μM due to the dilution effect of adding the DDP.

### Example 4

Analysis of whether A6 differentially modulates the sensitivity of cisplatin-sensitive versus cisplatin resistant human ovarian cancer IGROV-1 cells to cisplatin was tested.

Study Conditions. Drugs: DDP (3.33 mM stock) and A6 (109.8 mM stock); Drug exposure time: 1 hour; Cell line: IGROV-1 and its DDP-resistant subline IGROV-1/CP; Media: RPMI 1640 plus 10% FBS containing 1 mM penicillin/streptomycin; Cytotoxicity Assays: CCK-8 (Cell Counting Kit 8—Dojindo Molecular Technologies).

Tissue culture plates containing 96-well plates were seeded with 4000 cells in 100 μl of media per well. The wells on rows A-D were seeded with IGROV-1 cells, while the wells on rows E-H were seeded with IGROV-1/CP cells. The cells were allowed to attach overnight in a 37° C. in 5% CO₂ incubator. Approximately 24 h later medium was removed by aspiration and the cells were exposed to 0, 1, 10 or 100 μM A6 peptide in 100 μl. After 1 h, increasing concentrations of DDP were added to triplicate wells in 100 pa medium. Following a 1 h exposure to the combination of A6 and DDP, the drugs were removed by aspiration. The cells were then allowed to grow for 96 h following which the plates were assayed for relative cell growth with the CCK-8 assay.

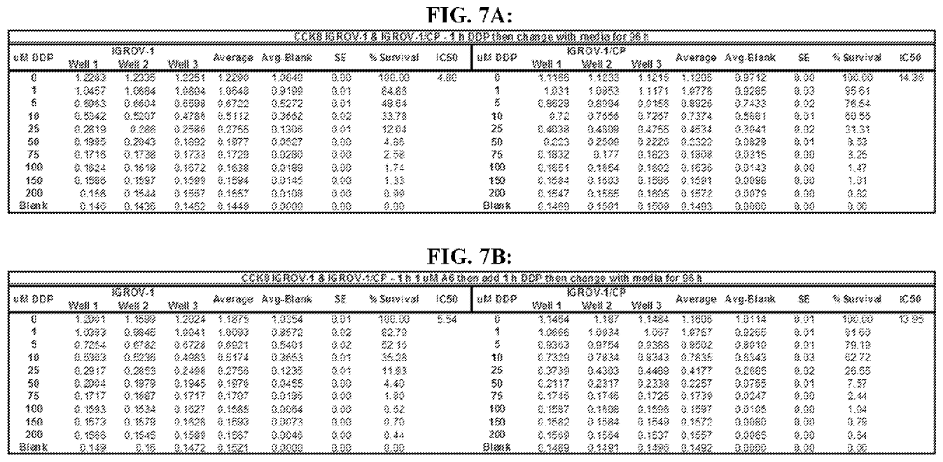

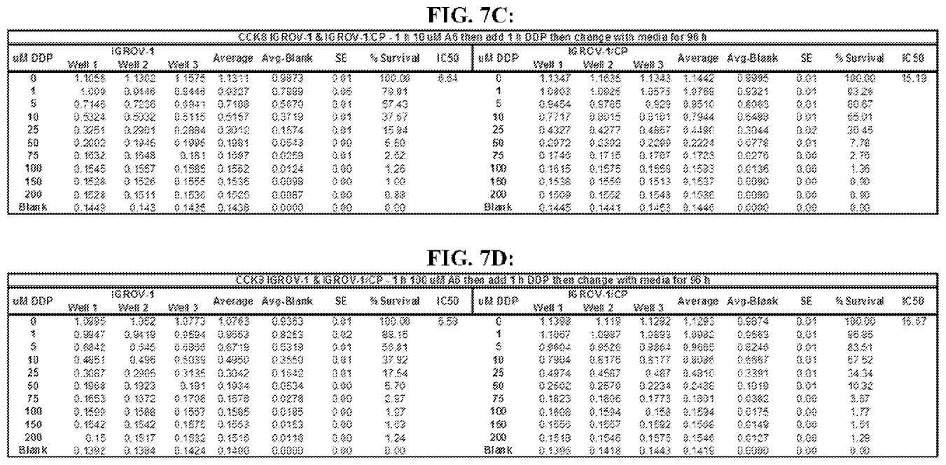

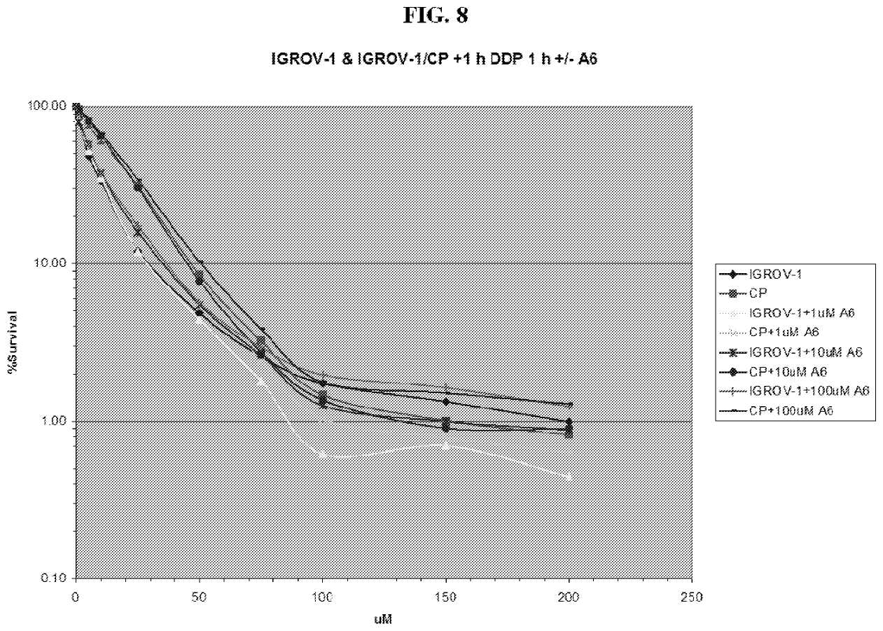

CCK-8 assay. Five pi of CCK-8 stain was added to each well and the plate was incubated at 37° C. incubator for 4 h. The plate was read with a microplate reader at a wavelength of 450 nM. (See FIGS. 7A-7D and FIG. 8).

The assay was performed using triplicate wells for each DDP concentration. The data in the tables below is the optical density in the well which is a function of the number of live cells remaining at the end of the 96 h growth period.

Under conditions where the IGROV-1 cells were growing in RPMI containing 10%, a 1 h exposure to A6 at concentration of 1, 10 or 100 μM followed by a 1 h exposure to both A6 and DDP failed to modulate the sensitivity of either the DDP-sensitive IGROV-1 cells, or the DDP-resistant IGROV-1/CP cells to DDP. IGROV-1/CP cells appeared not to be resistant to DDP. The concentration of A6 during the first hour of exposure was 1, 10 or 100 μM. During the second hour of exposure, when the DDP was present, the concentration of A6 was 0.5, 5 or 50 μM due to the dilution effect of adding the DDP.

### Example 5

The effect of A6 plus Cisplatin (DDP) or Paclitaxel (PTX) in the B16F10-DsRed Cell Lung Metastasis Model was tested.

B16F10-DsRed cells were harvested from cells culture. 1×10⁶ cells/200 μl were inoculated into DPBS/mouse via bilateral tail vein in 48 female C57BL/6 mice (Charles River).

Mice were randomized into six groups: Grp1 no treatment, Grp2 PTX (10 mg/Kg, IP on days 1 and 8), Grp3 DDP (4 mg/Kg, IP, day 1, 4 and 8), Grp4 A6 (200 mg/Kg, SC, QD), Grp5 A6 plus PTX, Grp6 A6 plus DDP. Animals were weighed before dosing and then twice a week.

Dosing: A6 dosing started immediately after cell inoculation and consecutively for 11 days; DDP/PTX dosing started immediately after inoculation and then on days as above. The study was ended on day 12, and the lungs were harvested to determine metastatic burden.

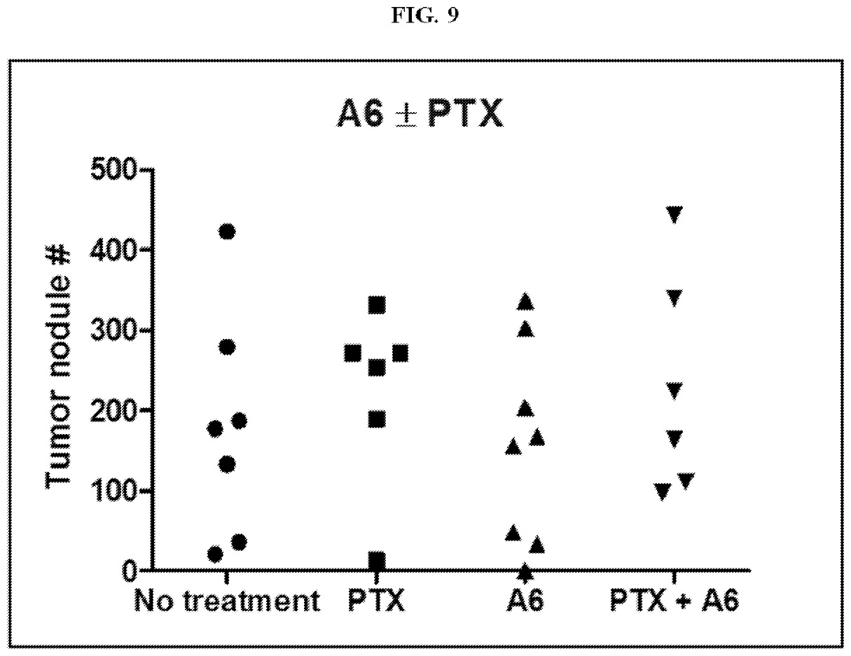

A6±Paclitaxel (PTX). Tumor burden in the lungs was not significantly reduce by A6 in the presence or absence of paclitaxel. (FIG. 9)

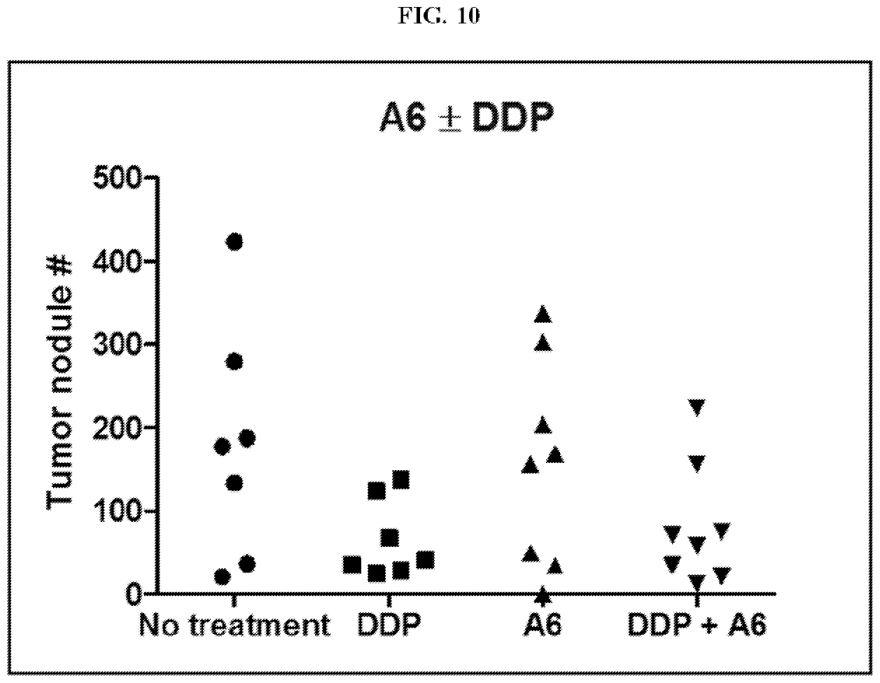

A6±Cisplatin (DDP). Tumor burden in the lungs was not significantly reduced by A6 in the presence or absence of cisplatin. (FIG. 10)

Compared to the no treatment group, no treatment significantly reduced the number of tumor nodules in the lung. Furthermore, the addition of A6 did not increase antimetastatic activity of cisplatin or paclitaxel.

### Example 6

Effect of A6 plus Cisplatin (DDP) or Paclitaxel (PTX) in HEY (DDP sensitive) and HEY/C2 (DDP resistant) cells was tested. The cells were plated in 96-well plates as described and incubated overnight. The cells were treating with various doses of DDP/PTX in the absence or presence of 100 μM A6 in culture medium containing 10% FBS and incubated for 72 hours. The CCK-8 assay described herein was performed to access cytotoxicity of DDP/PTX.

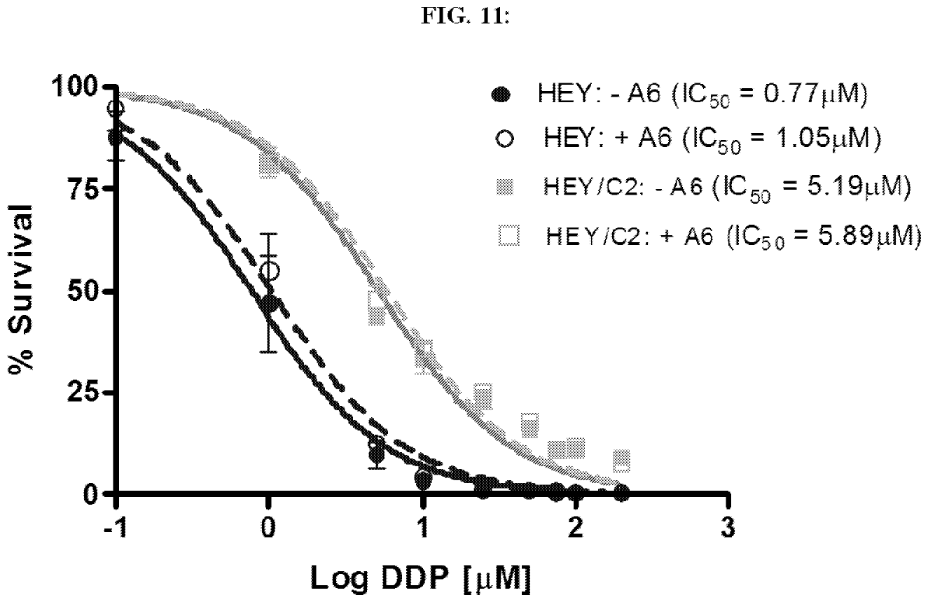

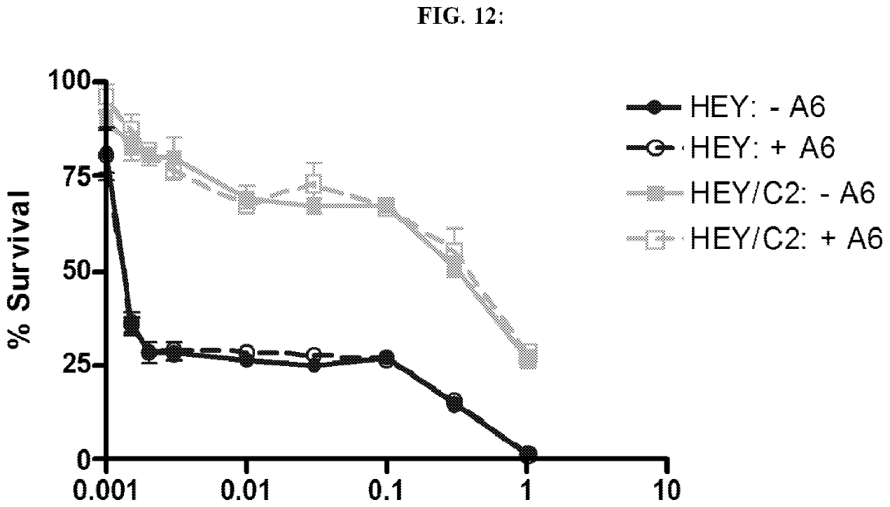

HEY/C2 cells were resistant to DDP with about 7 fold higher IC₅₀ compared to HEY cells. A6 did not affect sensitivity of HEY or HEY/C2 cells to cisplatin. DDP-resistant HEY/C2 cells were also resisted to paclitaxel compared to DDP-sensitive HEY cells. A6 did not affect sensitivity of HEY or HEY/C2 cells to paclitaxel. The dose-response curve appears as biphasic, indicating a possibility of the presence of a relatively resistant cell subpopulation. (See FIGS. 11 and 12).

### Example 7

A6 was administered in combination with paclitaxel as a compassionate use. The patient (age 65) was diagnosed with breast cancer after mammogram detected a mass. A lumpectomy performed was performed resulting in removal of a 19 cm tumor.

The patient was started on adjuvant chemotherapy consisting of Cytoxan (880 mg) and Adriamycin (89 mg) q 3 weeks ×4 followed by radiation therapy to the left breast to a total dose of 4500 cGy.

The patient returned with progressively increasing swelling, redness of skin, tenderness of left breast. Core biopsy revealed diffuse infiltrating ductal carcinoma consistent with recurrence of primary tumor. Breast tissue nearly replaced by tumor—considered rapidly growing and refractory to chemotherapy. Whole body CAT scan and bone scan revealed no identifiable metastases. The patient was administered Taxol, at 268 mg daily for 2 weeks. The patient was started on A6 150 mg/day and continued on this regimen through 9 years of daily treatment. A6 treatment discontinued after 9 years of biannual testing showed no evidence of disease. Patient in complete remission with no clinical evidence of distant metastasis or local recurrence of the cancer.

A second compassionate use patient was administered A6. The patient (age 58) was diagnosed with endometrial cancer with metastases to lymph nodes and lungs. The patient underwent a hysterectomy followed by cisplatin and paclitaxel regimen. The patient started A6 1.5 mg/kg/day and continued on this regimen for 2 years with intermittent radiation therapy to the mediastinum to a total dose of 5,000 cGy. Stable disease was identified and the A6 dose reduced to 0.25-0.5 mg/kg/day. CT scan revealed non-calcified nodules in right lung. CA-125 increased from 25 to 71 U/ml. A6 increased to 2.0 mg/kg/day.

A follow up CT scan revealed no nodules. CA-125 levels reduced to 22 U/ml. Ca-125 remained below 25 U/ml and all subsequent CT scans and PET scans have shown no evidence of disease. A6 treatment discontinued after 10+ years of biannual testing showed no evidence of disease.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

1.-63. (canceled)

64. A CD44-modulating polypeptide for treating cancer in a patient, inhibiting metastasis of a cancer in a patient, or restoring anti-cancer activity of an anti-cancer agent having reduced or eliminated activity against one or more cancers, wherein said method comprises administering the CD44-modulating polypeptide in combination with an anti-cancer agent, wherein the anti-cancer agent is selected from a group consisting of radiation therapy, a taxane, a PARP inhibitor, an anthracycline, or a combination thereof.

65. The CD44-modulating polypeptide of claim 64, wherein said taxane comprises paclitaxel, paclitaxel analogues, docetaxel, or cabaziltaxel, wherein said PARP inhibitor comprises talazoparib, olaparib, or rucaparib, and wherein said anthracycline comprises daunorubicin, doxorubicin.

66. The CD44-modulating polypeptide of claim 64, wherein the cancer is resistant or refractory cancer, wherein the cancer is resistant to at least one anti-cancer agent.

67. The CD44-modulating polypeptide of claim 64, wherein said CD44-modulating polypeptide is a polypeptide comprising SEQ ID NO:1, SEQ ID NO:2, or a variant sequence of SEQ ID NO:1, wherein the variant sequence comprises one or more amino acid mutation with respect to SEQ ID NO: 1 selected from:
(i) K¹ to A;
(ii) P², P⁵, P⁶, or a combination thereof to A;
(iii) S³, S⁴, or S³ and S⁴ to A; or
(iv) E⁷, E⁸, or E⁷ and E⁸ to A,
wherein said mutation retains activity about equal to or greater than a polypeptide of SEQ ID NO:1.

68. The CD44-modulating polypeptide of claim 64, wherein said cancer is a solid tumor or hematological cancer, or wherein said cancer is selected from ovarian cancer, breast cancer, colorectal cancer, prostate cancer, head and neck cancer, endometrial cancer, primary peritoneal cancer, liver cancer, or glioblastoma, optionally wherein said breast cancer is resistant or refractory to treatment with radiation therapy.

69. The CD44-modulating polypeptide of claim 64, wherein said CD44-modulating polypeptide is administered:
(i) QD or BID;
(ii) continuously and without a rest period;
(iii) or transdermal administration, optionally wherein said transdermal administration is performed using an immediate release transdermal device, or sustained release transdermal device; and/or
(iv) by intranasal administration.

70. The CD44-modulating polypeptide of claim 64, wherein said CD44-modulating polypeptide is administered at an amount of about 100 mg to about 500 mg.

71. The CD44-modulating polypeptide of claim 64, wherein said radiation therapy is selected from the group consisting of: X-Rays, gamma-Rays, UV-Rays, a particle beam, and decay of a radioactive isotope.

72. The CD44-modulating polypeptide of claim 71, wherein said particle beam is selected from the group consisting of: an electron beam, a neutron beam, a proton beam, a carbon ion beam, and a pion beam.

73. The CD44-modulating polypeptide for use of claim 71, wherein said radioactive isotope is a radioactive isotope of iodine, preferably iodine 125 or iodine 131, strontium, phosphorous, palladium, cesium, iridium, phosphate, samarium, yttrium, cobalt, preferably cobalt 60.

74. A method of treating cancer in a patient, inhibiting metastasis of a cancer in a patient, or restoring anti-cancer activity of an anti-cancer agent having reduced or eliminated activity against one or more cancers, wherein said method comprises administering a CD44-modulating polypeptide in combination with an anti-cancer agent, wherein the anti-cancer agent is selected from a group consisting of a radiation therapy, a taxane, a PARP inhibitor, an anthracycline, or a combination thereof.

75. The method of claim 74, wherein said taxane comprises paclitaxel, paclitaxel analogues, docetaxel, or cabaziltaxel, wherein said PARP inhibitor comprises talazoparib, olaparib, or rucaparib, wherein said anthracycline comprises daunorubicin, doxorubicin.

76. The method of claim 74, wherein the cancer is resistant or refractory cancer, wherein the cancer is resistant to at least one anti-cancer agent.

77. The method of claim 74, wherein said CD44-modulating polypeptide is a polypeptide comprising SEQ ID NO:1, SEQ ID NO:2, or a variant sequence of SEQ ID NO:1, wherein the variant sequence comprises one or more amino acid mutation with respect to SEQ ID NO: 1 selected from:
(i) K¹ to A;
(ii) P², P⁵, P⁶, or a combination thereof to A;
(iii) S³, S⁴, or S³ and S⁴ to A; or
(iv) E⁷, E⁸, or E⁷ and E⁸ to A,
wherein said mutation retains activity about equal to or greater than a polypeptide of SEQ ID NO:1.

78. The method of claim 74, wherein said cancer is a solid tumor or hematological cancer, or wherein said cancer is selected from ovarian cancer, breast cancer, colorectal cancer, prostate cancer, head and neck cancer, endometrial cancer, primary peritoneal cancer, liver cancer, or glioblastoma, optionally wherein said breast cancer is resistant or refractory to treatment with radiation therapy.

79. The method of claim 74, wherein said CD44-modulating polypeptide is administered:
(i) QD or BID;
(ii) continuously and without a rest period;
(iii) or transdermal administration, optionally wherein said transdermal administration is performed using an immediate release transdermal device, or sustained release transdermal device; and/or
(iv) by intranasal administration.

80. The method of claim 74, wherein said CD44-modulating polypeptide is administered at an amount of about 100 mg to about 500 mg.

81. The method of claim 74, wherein said radiation therapy is selected from the group consisting of: X-Rays, gamma-Rays, UV-Rays, a particle beam, and decay of a radioactive isotope.

82. The method of claim 81, wherein said particle beam is selected from the group consisting of: an electron beam, a neutron beam, a proton beam, a carbon ion beam, and a pion beam.

83. The method of claim 81, wherein said radioactive isotope is a radioactive isotope of iodine, preferably iodine 125 or iodine 131, strontium, phosphorous, palladium, cesium, iridium, phosphate, samarium, yttrium, cobalt, preferably cobalt 60.


SEQUENCE LISTING (6 sequences)

SEQ ID NO: 1 - protein, 8 aa, Artificial Sequence
  A6
  MOD_RES (1)..(1): ACETYLATION
  MOD_RES (8)..(8): AMIDATION
KPSSPPEE

SEQ ID NO: 2 - protein, 8 aa, Artificial Sequence
  CD44 Sequence Homolog
  MOD_RES (1)..(1): ACETYLATION
  MOD_RES (8)..(8): AMIDATION
NASAPPEE

SEQ ID NO: 3 - protein, 14 aa, Artificial Sequence
  CD44-modulating polypeptide 3
QETWFQNGWQGKNP

SEQ ID NO: 4 - protein, 14 aa, Artificial Sequence
  CD44-modulating polypeptide 4
KEKWFENEWQGKNP

SEQ ID NO: 5 - protein, 14 aa, Artificial Sequence
  CD44-modulating polypeptide 5
KEQWFGNRWHEGYR

SEQ ID NO: 6 - protein, 24 aa, Artificial Sequence
  CD44-modulating polypeptide 6
QIRQQPRDPPTETLELEVSPDPAS